(19) United States
(12) Patent Application Publication
(10) Pub. No.: US 2008/0300135 A1
(43) Pub. Date: Dec. 4, 2008

(54) 5-Alkyl-7-Amino-6-Heteroaryl-1,2,4-Triazolo[1,5-A]Pyrimidine Compounds and Their Use for Controlling Harmful Fungi

(75) Inventors: Oliver Wagner, Neustadt (DE); Sarah Ulmschneider, Mainz (DE); Udo Hunger, Mannheim (DE)

Correspondence Address: HUTCHISON LAW GROUP PLLC, PO BOX 31686, RALEIGH, NC, 27612, US

(73) Assignee: BASF Aktiengesellschaft, Ludwigshafen (DE)

(21) Appl. No.: 11/995,454

(22) PCT Filed: Jul. 6, 2006
(86) PCT No.: PCT/EP06/63970

§ 371 Date: Jan. 11, 2008

(30) Foreign Application Priority Data
Jul. 13, 2005 (DE) 10 2005 033 143.2
Jul. 29, 2005 (DE) 10 2005 036 319.9

(51) Int. Cl. A01N 43/90; C07D 487/04; A01P 3/00

(52) U.S. Cl. 504/100; 544/263; 514/259.31

(57) ABSTRACT

The present invention relates to 7-amino-6-heteroaryl-1,2,4-triazolo[1,5-a]pyrimidine compounds of the formula (I),

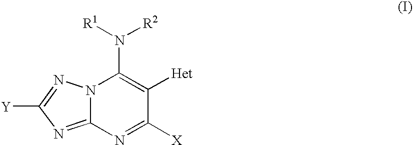

in which the substituents Het, R¹, R², X and Y are as defined in the claims and in the description.

The present invention relates to 7-amino-6-heteroaryl-1,2,4-triazolo[1,5-a]pyrimidine compounds of the formula (I)

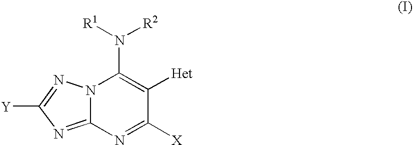

in which the substituents Het, R¹, R², X and Y are as defined below:
Het is a five-membered aromatic heterocycle which contains one, two, three or four heteroatoms from the group consisting of oxygen, nitrogen and sulfur, where Het is unsubstituted or substituted by one, two, three or four identical or different substituents L:
   L is halogen, cyano, nitro, hydroxyl, cyanato (OCN), (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₄-C₁₀)-alkadienyl, (C₄-C₁₀)-haloalkadienyl, (C₁-C₈)-alkoxy, (C₁-C₈)-haloalkoxy, (C₂-C₈)-alkenyloxy, (C₂-C₈)-haloalkenyloxy, (C₂-C₈)-alkynyloxy, (C₂-C₈)-haloalkynyloxy, (C₃-C₈)-cycloalkyl, (C₃-C₈)-halocycloalkyl, (C₃-C₈)-cycloalkenyl, (C₃-C₈)-halocycloalkenyl, (C₃-C₈)-cycloalkoxy, (C₁-C₈)-alkoximino-(C₁-C₈)-alkyl, (C₂-C₈)-alkenyloximino-(C₁-C₈)-alkyl, (C₂-C₈)-alkynyloximino-(C₁-C₈)-alkyl, S(═O)ₙA¹, C(═O)ₙA², C(═S)A², NR⁵R⁶, NR⁵—(C═O)—R⁶; where
      n is 0, 1 or 2;
      A¹ is hydrogen, hydroxyl, (C₁-C₈)-alkyl, (C₁-C₈)-haloalkyl, amino, (C₁-C₈)-alkylamino or di-(C₁-C₈)-alkylamino, A² is one of the groups mentioned under A¹ or (C₂-C₈)-alkenyl, (C₂-C₈)-haloalkenyl, (C₂-C₈)-alkynyl, (C₂-C₈)-haloalkynyl, (C₁-C₈)-alkoxy, (C₁-C₈)-haloalkoxy, (C₂-C₈)-alkenyloxy, (C₂-C₈)-haloalkenyloxy, (C₂-C₈)-alkynyloxy, (C₂-C₈)-haloalkynyloxy, (C₃-C₈)-cycloalkoxy, (C₃-C₈)-halocycloalkoxy;
      R⁵, R⁶ independently of one another are hydrogen, (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₃-C₈)-cycloalkyl, (C₃-C₈)-halocycloalkyl, (C₃-C₈)-cycloalkenyl or (C₃-C₈)-halocycloalkenyl,
      where the aliphatic groups of the radical definitions of L for their part may carry one to four identical or different groups R^{L};
         R^{L} is halogen, cyano, (C₁-C₈)-alkoxy, (C₂-C₈)-alkenyloxy, (C₂-C₈)-alkynyloxy, (C₃-C₈)-cycloalkyl, (C₃-C₈)-cycloalkenyl, (C₃-C₈)-cycloalkyloxy, (C₃-C₈)-cycloalkenyloxy, (C₁-C₈)-alkoximino-(C₁-C₈)-alkyl, (C₂-C₈)-alkenyloximino-(C₁-C₈)-alkyl, S(═ₙA¹, C₂-C₈) -alkynyloximino-(C₁-C₈-alkyl, S(═O)ₙA¹, C(═O)ₙA¹, C(═S)A NR⁵R⁶, NR⁵—(C═O)—R;
   R¹, R² independently of one another are hydrogen, (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₄-C₁₀)-alkadienyl, (C₄-C₁₀)-haloalkadienyl, (C₁-C₈)-alkoxy, (C₁-C₈)-haloalkoxy, (C₂-C₈)-alkenyloxy, (C₂-C₈)-haloalkenyloxy, (C₂-C₈)-alkynyloxy, (C₂-C₈)-haloalkynyloxy, (C₃-C₈)-cycloalkyl, (C₃-C₈)-halocycloalkyl, (C₃-C₈)-cycloalkenyl, (C₃-C₈)-halocycloalkenyl, (C₃-C₈)-cycloalkoxy, (C₃-C₈)-halocycloalkoxy, (C₅-C₁₀)-bicycloalkyl, amino, (C₁-C₈)-alkylamino, di-(C₁-C₈)-alkylamino, phenyl, naphthyl or a five- or six-membered saturated, partially unsaturated or aromatic heterocycle which contains one, two, three or four heteroatoms from the group consisting of O, N and S;
   R¹ and R² together with the nitrogen atom to which they are attached may also form a five- or six-membered saturated, partially unsaturated or aromatic heterocycle which may contain one, two or three further heteroatoms from the group consisting of O, N and S as ring members, where
   R¹ and/or R² or a heterocycle formed from R¹ and R² may carry one, two, three or four identical or different groups R^{a} and/or two substituents attached to adjacent ring atoms may represent (C₁-C₆)-alkylene, oxy-(C₂-C₄)-alkylene or oxy-(C₁-C₃)-alkyleneoxy; where R^{a} is as defined below:
      R^{a} is halogen, cyano, nitro, hydroxyl, carboxyl, amino, (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₄-C₁₀)-alkadienyl, (C₁-C₈)-alkoxy, (C₁-C₈)-haloalkoxy, (C₂-C₈)-alkenyloxy, (C₂-C₈)-haloalkenyloxy, (C₂-C₈)-alkynyloxy, (C₂-C₈)-haloalkynyloxy, (C₃-C₈)-cycloalkyl, (C₃-C₈)-halocycloalkyl, (C₃-C₈)-cycloalkenyl, (C₃-C₈)-cycloalkoxy, (C₃-C₈)-halocycloalkoxy, (C₃-C₈)-cycloalkenyloxy, (C₅-C₁₀)-bicycloalkyl, (C₁-C₈)-alkylcarbonyl, (C₁-C₈)-alkoxycarbonyl, (C₁-C₈)-alkylcarbonyloxy, (C₁-C₈)-alkylamino, di-(C₁-C₈)-alkylamino, (C₁-C₈)-alkylaminocarbonyl, di-(C₁-C₈)-alkylaminocarbonyl, (C₁-C₈)-alkylaminothiocarbonyl, di-(C₁-C₈)-alkylaminothiocarbonyl, oxy-(C₁-C₆)-alkyleneoxy, (C₁-C₈)-alkylthio, (C₁-C₈)-alkylsulfinyl, (C₁-C₅)-alkylsulfonyl, (C₁-C₈)-alkylsulfoxyl, phenyl, naphthyl, a five- to ten-membered saturated, partially unsaturated or aromatic heterocycle which contains one, two, three or four heteroatoms from the group consisting of O, N and S;
      where the aliphatic, alicyclic or aromatic groups in R^{a} for their part may carry one, two or three identical or different groups R^{b}:
         R^{b} is halogen, cyano, nitro, hydroxyl, carboxyl, mercapto, amino, formyl, aminocarbonyl, aminothiocarbonyl, (C₁-C₈)-alkyl, (C₁-C₈)-haloalkyl, (C₂-C₈)-alkenyl, (C₂-C₈)-haloalkenyl, (C₄-C₁₀)-alkadienyl, (C₂-C₈)-alkynyl, (C₂-C₈)-haloalkynyl, (C₁-C₈)-alkoxy, (C₁-C₈)-haloalkoxy, (C₂-C₈)-alkenyloxy, (C₂-C₈)-alkynyloxy, (C₃-C₈)-cycloalkyl, (C₃-C₈)-cycloalkenyl, (C₃-C₈)-cycloalkoxy, (C₃-C₈)-cycloalkenyloxy, (C₅-C₁₀)-bicycloalkyl, amino, (C₁-C₈)-alkylamino, di-(C₁-C₈)-alkylamino, (C₁-C₈)-alkylcarbonyl, (C₁-C₈)-alkoxycarbonyl, (C₁-C₈)-alkylcarbonyloxy, (C₁-C₈)-alkylaminocarbonyl, di-(C₁-C₈)-alkylaminocarbonyl, (C₁-C₈)-alkylaminothiocarbonyl, di-(C₁-C₈)-alkylaminothiocarbonyl, (C₁-C₈)-alkylthio, (C₁-C₈)-alkylsulfinyl, (C₁-C₈)-alkylsulfonyl, (C₁-C₈)-alkylsulfoxyl, (C₆-C₁₄)-aryl, (C₆-C₁₄)-haloaryl, (C₆-C₁₄)-aryloxy, (C₆-C₁₄)-arylthio, (C₆-C₁₄)-aryl-(C₁-C₆)-alkoxy, (C₆-C₁₄)-aryl-(C₁-C₆)-alkyl, a five- to ten-membered saturated, partially unsaturated or aromatic heterocycle which contains one, two, three or four heteroatoms from the group consisting of O, N and S, heterocyclyloxy, heteroaryloxy, heteroarylthio, where the cyclic systems may be partially or fully halogenated and/or substituted by (C₁-C₈)-alkyl and/or (C₁-C₈)-haloalkyl groups;
   X is (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl; cyano-(C₁-C₄)-alkyl, (C₁-C₄)-alkoxy-(C₁-C₄)-alkyl;
   Y is hydrogen, halogen, cyano, (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₁-C₈)-alkoxy, (C₁-C₈)-haloalkoxy, (C₃-C₈)-cycloalkyl, (C₃-C₈)-halocycloalkyl, (C₁-C₈)-alkylthio, (C₁-C₈)-alkylsulfinyl or (C₁-C₈)-alkylsulfonyl;
   and agriculturally acceptable salts thereof.

Furthermore, the present invention relates to compositions comprising at least one of the compounds according to the invention, to processes for preparing these compounds, to intermediates for preparing the compounds and the agriculturally acceptable salts thereof, to the preparation of the intermediates and to the use of the compounds according to the invention for controlling phytopathogenic fungi.

Depending on the substitution pattern, the compounds of the formula (I) may have one or more centers of chirality, in which case they are present as enantiomer or diastereomer mixtures. The invention provides both the pure enantiomers or diastereomers or rotamers and mixtures thereof. Suitable compounds of the formula (I) also include all possible stereoisomers (cis/trans isomers) and mixtures thereof. The compounds according to the invention and/or their salts can be present in different crystal modifications, which may differ from one another in biological activity. They also form part of the subject matter of the present invention.

7-Amino-6-heteroaryl-1,2,4-triazolo[1,5a]pyrimidines and their use in the field of the control of microorganisms such as harmful fungi are known per se.

EP-A 613 900 relates to 7-amino-1,2,4-triazolo[1,5-a]pyrimidine compounds and their use as fungicides, where the compounds contain a hydrogen atom, a halogen atom or an amino group in the 5-position. In the 6-position, there is an optionally substituted cycloalkyl ring or a heterocyclic group, a heterocyclic group being, according to EP 0 613 900, a 3- to 6-membered, preferably a 5- or 6-membered, ring system.

Intermediates of the formula (II) which are used for preparing fungicidally active triazolopyrimid-7-ylideneamines are known from WO 01/96341. In position 5, the intermediates may contain a halogen atom, an amino group or an alkoxy group. In position 6 there is a phenyl, cycloalkyl or a five- or six-membered heteroaryl group.

Intermediates of the formula (II) which are used for preparing fungicidally active 2-(cyanoamino)pyrimidines are known from WO 01/96314. In position 5, these compounds carry a hydrogen atom, a halogen atom, an alkyl, alkoxy, alkylthio or alkylamino group, preferably chloride. In position 6 there is a phenyl, cycloalkyl or a 5- or 6-membered heteroaryl group.

WO 04/011467 relates to 1,2,4-triazolo[1,5-a]pyrimidine compounds which carry a halogen atom, a cyano, alkoxy, alkylthio, alkylsulfenyl, alkylsulfonyl or alkoxycarbonyl group in position 5. In the 6-position, there is a 5- or 6-membered heterocyclyl group which may be optionally substituted pyrrolyl, thienyl, pyrazolyl, imidazolyl, oxazolyl, isoxazolyl, thiazolyl, isothiazolyl or pyrimidinyl.

WO 04/108727 discloses 1,2,4-triazolo[1,5-a]pyrimidines and their use for controlling unwanted microorganisms. In position 5, these compounds have exclusively halogen radicals; position 6 of the pyrimidine ring is substituted either by pyridyl or pyrimidyl radicals.

WO 04/113342 relates to 1,2,4-triazolo[1,5-a]pyrimidines which are substituted in the 2-position of the 1,2,4-triazolo[1,5-a]pyrimidine skeleton and may only carry a halogen group in position 5. In position 6, there is a 5- or 6-membered heterocyclyl radical having 1 to 4 heteroatoms such as nitrogen, oxygen and/or sulfur, pyridyl, pyrimidyl, thienyl and thiazolyl being preferred heterocyclyl radicals.

With respect to their fungicidal action, the 1,2,4-triazolo[1,5-a]pyrimidines known from the prior art are not entirely satisfactory, or they have unwanted properties, such as poor compatibility with crop plants.

Accordingly, it is an object of the present invention to provide novel compounds having improved fungicidal activity and/or better compatibility with crop plants.

Surprisingly, this object is achieved by the compounds according to the invention and/or by the agriculturally acceptable salts of the compounds according to the invention.

According to the present invention, agriculturally acceptable salts include in particular the salts of those cations and the acid addition salts of those acids whose cations and anions, respectively, have no adverse effect on the fungicidal action of the compounds according to the invention.

Thus, suitable cations are in particular the ions of the alkali metals, preferably sodium or potassium, of the alkaline earth metals, preferably calcium, magnesium or barium, and of the transition metals, preferably manganese, copper, zinc or iron, or also the ammonium ion which, if desired, may carry from one to four (C₁-C₄)-alkyl substituents and/or one phenyl or benzyl substituent, preferably diisopropylammonium, tetramethylammonium, tetrabutylammonium, trimethylbenzylammonium, and also phosphonium ions, sulfonium ions, preferably tri(C₁-C₄-alkyl)sulfonium, and sulfoxonium ions, preferably tri(C₁-C₄-alkyl)sulfoxonium.

Anions of acid addition salts which can be employed advantageously are, for example, chloride, bromide, fluoride, hydrogen sulfate, sulfate, dihydrogenphosphate, hydrogenphosphate, phosphate, nitrate, bicarbonate, carbonate, hexafluorosilicate, hexafluorophosphate, benzoate, and also the anions of (C₁-C₄)-alkanoic acids, preferably formate, acetate, propionate and butyrate. They can be formed by reaction of the compounds of the formula (I) according to the invention with an acid of the corresponding anion, preferably hydrochloric acid, hydrobromic acid, sulfuric acid, phosphoric acid or nitric acid.

The compounds of the formula (I) according to the invention can be obtained by various routes analogously to processes, known per se, of the prior art. The compounds according to the invention can be prepared, in particular, as follows:

Compounds of the formula (I) can be prepared, for example, by reacting a 7-halotriazolopyrimidine of the formula (II)

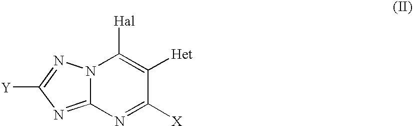

with an amine HNR¹R², where Hal is halogen and Het, X, Y, R¹ and R² are as defined for compounds of the formula (I). In particular for X═(C₁-C₄)-alkoxy-(C₁-C₄)-alkyl and cyano-(C₁-C₄)-alkyl see also Pharmazie 33, 1978, 42.

The reaction of the 7-halotriazolopyrimidine of the formula (II) with alkylamines is carried out analogously to the prior art cited at the outset or analogously to the methods described in WO 98/46608.

Advantageously, the process is carried out at temperatures in the range from 0° C. to 70° C., preferably from 10° C. to 35° C.

The reaction is preferably carried out in an inert solvent, for example an ether, such as, for example, dioxane, diethyl ether, diisopropyl ether, tert-butyl methyl ether or, in particular, tetrahydrofuran, a halogenated hydrocarbon, such as dichloromethane or dichloroethane, or an aromatic hydrocarbon, such as, for example, toluene or o-, m-, p-xylene, or in a mixture of the solvents mentioned above.

Preference is furthermore given to using a base, such as, for example, tertiary amines, in particular triethylamine, biscyclohexylmethylamine, pyridine, picoline or inorganic bases, such as potassium carbonate. It is also possible for excess amine HNR¹R² to serve as base.

The amines HNR¹R² used in this process are generally commercially available or can be prepared by processes generally known to the person skilled in the art.

The present invention furthermore provides compounds of the formula (II)

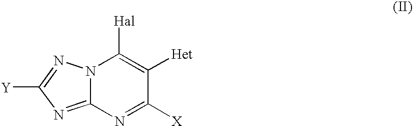

in which Hal is halogen and Het, X and Y are as defined for compounds of the formula (I). Hal is preferably chlorine or bromine. Particularly preferred compounds of the formula (I) according to the invention can be obtained from compounds of the formula (II) in which Het, X and Y are as defined in Tables 1 to 156.

7-Halotriazolopyrimidines of the formula (II) can be obtained by reacting the corresponding 7-hydroxytriazolopyrimidine of the formula (III)

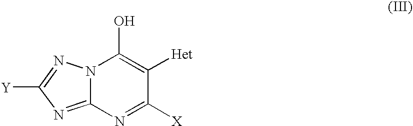

with a halogenating agent, where Het, X and Y are as defined for compounds of the formula (I). See also Pharmazie 33, 1978, 42.

The halogenation is carried out analogously to the prior art cited at the outset or according to the methods described in WO-A 94/20501.

The halogenating agent used is advantageously a phosphorus oxyhalide or a phosphorus (V) halide, such as phosphorus pentachloride, phosphorus oxybromide or phosphorus oxychloride or a mixture of phosphorus oxychloride and phosphorus pentachloride.

The reaction of the compounds of the formula (III) with the halogenating agent is usually carried out at from 0° C. to 150° C., preferably from 80° C. to 125° C. [cf. also EP-A 770 615].

The reaction can be carried out in the absence of a solvent or in an inert solvent, for example a halogenated hydrocarbon, such as dichloromethane or dichloroethane, or an aromatic hydrocarbon, such as, for example, toluene or o-, m-, p-xylene or in a mixture of the solvents mentioned.

The present invention furthermore provides compounds of the formula (III)

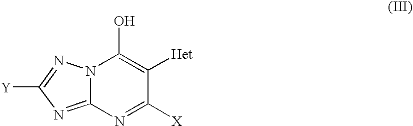

in which Het, X and Y are as defined for compounds of the formula (I). Particularly preferred compounds of the formula (I) or (II) according to the invention can be obtained from compounds of the formula (III) in which Het, X and Y are as defined in Tables 1 to 156.

7-Hydroxytriazolopyrimidines of the formula (III) can be prepared analogously to the methods described in Adv. Het. Chem. Vol. 57, p. 81ff. (1993).

Compounds of the formula (III) can be obtained by reacting a compound of the formula (IV)

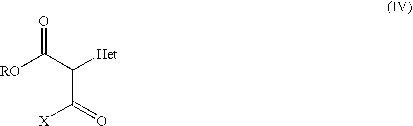

with a triazole of the formula (V)

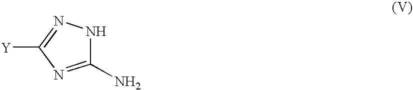

where Het, X and Y are as defined for compounds of the formula (I) and R is alkyl, preferably (C₁-C₆)-alkyl, more preferably (C₁-C₄)-alkyl, in particular methyl or ethyl. X is preferably (C₁-C₈)-alkyl, (C₂-C₈)-alkenyl, (C₂-C₈)-alkynyl, a correspondingly halogenated radical or (C₁-C₄)-alkoxy-(C₁-C₄)-alkyl.

The reaction of a 3-amino-1,2,4-triazole (V) with a compound of the formula (IV) is usually carried out at temperatures of from 80° C. to 250° C., preferably from 120° C. to 180° C.

Preferably, the reaction is carried out without a solvent, or an inert organic solvent is used. The presence of a base may be preferred [cf. EP-A 770 615]. Furthermore, it may also be preferable to carry out the reaction in the presence of acetic acid under conditions generally known to the person skilled in the art.

Suitable solvents are, for example, aliphatic hydrocarbons, aromatic hydrocarbons, such as toluene, o-, m- and p-xylene, halogenated hydrocarbons, ethers, nitriles, ketones, alcohols, and also N-methylpyrrolidone, dimethyl sulfoxide, dimethylformamide and dimethylacetamide.

With particular preference, the reaction is carried out without solvent or in chlorobenzene, xylene, dimethyl sulfoxide or N-methylpyrrolidone. It is also possible to use mixtures of the solvents mentioned. If appropriate, catalytic amounts of acids, such as p-toluenesulfonic acid, acetic acid or propionic acid, may be added, too.

Suitable bases are, in general, inorganic compounds, such as alkali metal and alkaline earth metal hydroxides, alkali metal and alkaline earth metal oxides, alkali metal and alkaline earth metal hydrides, alkali metal amides, alkali metal and alkaline earth metal carbonates, and also alkali metal bicarbonates, such as, for example, potassium carbonate, organometallic compounds, in particular alkali metal alkyls, alkylmagnesium halides, and also alkali metal and alkaline earth metal alkoxides and dimethoxymagnesium, moreover organic bases, for example tertiary amines, such as trimethylamine, triethylamine, truisopropylethylamine, tributylamine and N-methylpiperidine, N-methylmorpholine, pyridine, substituted pyridines, such as collidine, lutidine and 4-dimethylaminopyridine, and also bicyclic amines. Particular preference is given to using tertiary amines, such as triethylamine, triisopropylethylamine, tributylamine, N-methylmorpholine or N-methylpiperidine.

The bases are generally used in catalytic amounts; however, they can also be used in equimolar amounts, in excess or, if appropriate, as solvent.

In general, the starting materials are reacted with one another in equimolar amounts. In terms of yield, it may be advantageous to employ an excess of base and compound of the formula (IV), based on the 3-amino-1,2,4-triazole of the formula (V).

The present invention furthermore provides compounds of the formula (IV)

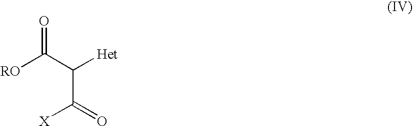

in which Het and X are as defined for compounds of the formula (I) and R is alkyl, preferably (C₁-C₆)-alkyl, more preferably (C₁-C₄)-alkyl, in particular methyl or ethyl. Particularly preferred compounds of the formula (I) or (III)/(II) according to the invention can be obtained from compounds of the formula (IV) in which Het and X are as defined in Tables 1 to 156.

Compounds of the formula (IV) can be prepared analogously to standard processes in the sense of a mixed ester condensation from the corresponding hetarylacetic esters by reaction with the corresponding aliphatic (C₂-C₅)-carboxylic acid alkyl esters, such as ethyl acetate, ethyl propionate, ethyl butyrate or ethyl valerate, or with a reactive derivative thereof, for example an acid chloride or an acid anhydride, in the presence of a strong base, for example an alkoxide, an alkali metal amide or an organolithium compound, for example analogously to the methods described in J. Chem. Soc. Perkin Trans 1967, 767 or in Eur. J. Org. Chem. 2002, p. 3986.

Alternatively, the compounds of the formula (I) according to the invention in which R¹ and R² are hydrogen can also be prepared by reacting a ketonitrile of the formula (IV-1)

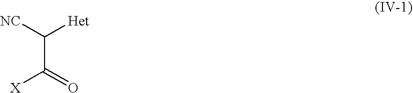

with a triazole of the formula (V), as shown above, where Het and X in formula (IV-1) have the meanings and preferred meanings mentioned for compounds of the formula (I) and X is preferably (C₁-C₈)-alkyl, (C₂-C₈)-alkenyl, (C₂-C₈)-alkynyl, a corresponding halogenated radical or (C₁-C₄)-alkoxy-(C₁-C₄)-alkyl.

The reaction can be carried out in the presence or absence of solvents. It is advantageous to use solvents which are substantially inert toward the starting materials and in which the starting materials are fully or partially soluble. Suitable solvents are in particular alcohols, such as ethanol, propanols, butanols, glycols or glycol monoethers, diethylene glycols or monoethers thereof, aromatic hydrocarbons, such as toluene, benzene or mesitylene, amides, such as dimethylformamide, diethylformamide, dibutylformamide, N,N-dimethylacetamide, lower alkanoic acids, such as formic acid, acetic acid, propionic acid, or bases, as mentioned above, and mixtures of these solvents with water. The reaction temperatures are between 50 and 300° C., preferably from 50 to 150° C., if the reaction is carried out in solution.

The compounds of the formula (I) are, if appropriate after evaporation of the solvent or dilution with water, isolated as crystalline compounds.

Some of the substituted alkyl cyanides of the formula (IV-1) required for this process are known, or they can be prepared analogously to known methods from alkyl cyanides and carboxylic esters using strong bases, for example alkali metal hydrides, alkali metal alkoxides, alkali metal amides or metal alkyls [cf.: J. Amer. Chem. Soc. Vol. 73, (1951) p. 3766]. See also Bioorganic & Medicinal Chemistry Letters (2004), 14(15), 3943-3947.

In an advantageous manner, the compounds of the formula (I) according to the invention can also be prepared by reacting compounds (IIa)

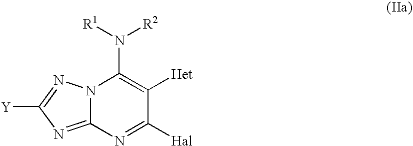

in which Hal is halogen, in particular chlorine or bromine, and Het, R¹, R² and Y are as defined for compounds of the formula (I), with an organometallic compound M-Z in which M is lithium, magnesium or zinc and Z is (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₁-C₄)-alkoxy-(C₁-C₄)-alkyl or cyano-(C₁-C₄)-alkyl. In this way, using the corresponding compounds M-Z, it is possible to prepare, in a particularly advantageous manner, compounds of the formula (I) in which X is (C₁-C₈)-alkyl, (C₂-C₈)-alkenyl or (C₂-C₈)-alkynyl.

The reaction is preferably carried out in the presence of catalytic or, in particular, at least equimolar amounts of transition metal salts and/or compounds, in particular in the presence of Cu salts, such as Cu(I) halides and, especially, Cu(I) iodide.

The reaction is preferably carried out in an inert organic solvent, for example one of the ethers mentioned above, in particular tetrahydrofuran, an aliphatic or cycloaliphatic hydrocarbon, such as hexane, cyclohexane and the like, an aromatic hydrocarbon, such as toluene, or in a mixture of these solvents.

The temperatures preferred for the reaction are in the range from −100 to +100° C., in particular in the range from −80° C. to +40° C. Processes for this purpose are known, for example from the prior art cited at the outset (see, for example, WO 03/004465).

Compounds of the formula (IIa) can be prepared by reacting a 5,7-dihalotriazolopyrimidine of the formula (IIb)

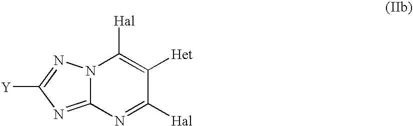

with the corresponding alkylamine HNR¹R². The conditions for this reaction correspond to those stated above for the reaction of compounds of the formula (II) with amines. Het and Y are here as defined for compounds (I).

5,7-Dihalotriazolopyrimidines of the formula (IIb) can be obtained, for example, by reacting the corresponding 5,7-dihydroxytriazolopyrimidine of the formula (IIc)

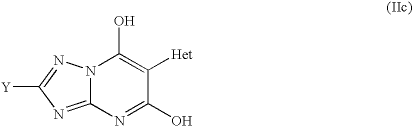

with a halogenating agent, analogously to the reaction described above. Het and Y are here as defined for compounds (I).

5,7-Dihydroxytriazolopyrimidines of the formula (IIc) can be prepared by various routes, for example analogously to the methods described in Adv. Het. Chem. Vol. 57, p. 81ff. (1993) or analogously to the prior art cited at the outset.

Compounds of the formula (I) in which X is (C₁-C₈)-alkyl can also be prepared by reacting, in a first step, a compound of the formula (IIa), as described above, with a malonate of the formula (IVa)

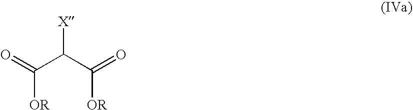

to give a compound of the formula (VI)

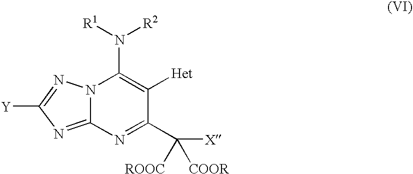

in which X″ is hydrogen or (C₁-C₇)-alkyl and R is (C₁-C₄)-alkyl and Het, R¹, R² and Y are as defined for compounds of the formula (I). The resulting compound of the formula (VI) is hydrolyzed and the hydrolysis product is decarboxylated [cf. U.S. Pat. No. 5,994,360].

The invention furthermore provides compounds of the formula (VI) in which X″ is hydrogen or (C₁-C₇)-alkyl and R is (C₁-C₄)-alkyl and Het, R¹, R² and Y are as defined for compounds of the formula (I).

Particularly preferred compounds of the formula (I) or (III)/(II) according to the invention can be obtained from compounds of the formula (VI) in which Het and X are as defined in Tables 1 to 156.

The malonates (IVa) are known from the literature, for example from J. Am. Chem. Soc., Vol. 64, 2714 (1942); J. Org. Chem., Vol. 39, 2172 (1974); Helv. Chim. Acta, Vol. 61, 1565 (1978)], or they can be prepared in accordance with the literature cited.

The subsequent hydrolysis of the ester (VI) is carried out under conditions generally known to the person skilled in the art. Depending on the various structural elements, alkaline or acidic hydrolysis of the compounds (VI) may be advantageous. Under the conditions of ester hydrolysis there may already be complete or partial decarboxylation to the compounds of the formula (I).

The decarboxylation is usually carried out at temperatures of from 20° C. to 180° C., preferably from 50° C. to 120° C.

The decarboxylation is preferably carried out in an inert solvent, if appropriate in the presence of an acid. Suitable acids are hydrochloric acid, sulfuric acid, phosphoric acid, formic acid, acetic acid, p-toluenesulfonic acid.

Suitable solvents are water, aliphatic hydrocarbons, such as pentane, hexane, cyclohexane and petroleum ether, aromatic hydrocarbons, such as toluene, o-, m- and p-xylene, halogenated hydrocarbons, such as methylene chloride, chloroform and chlorobenzene, ethers, such as diethyl ether, diisopropyl ether, tert-butyl methyl ether, dioxane, anisole and tetrahydrofuran, nitriles, such as acetonitrile and propionitrile, ketones, such as acetone, methyl ethyl ketone, diethyl ketone and tert-butyl methyl ketone, alcohols, such as methanol, ethanol, n-propanol, isopropanol, n-butanol and tert-butanol, and also dimethyl sulfoxide, dimethylformamide and dimethylacetamide; with particular preference, the reaction is carried out in hydrochloric acid or in acetic acid. It is also possible to use mixtures of the solvents mentioned.

The reaction mixtures obtained in the preparation of the compounds of the formula (I) or in the preparation of intermediates thereof can be worked up in the customary manner, for example by mixing with water, separating the phases and, if appropriate, chromatographic purification of the crude products. Some of the intermediates and end products are obtained in the form of colorless or slightly brownish viscous oils which can be purified or freed from volatile components under reduced pressure and at moderately elevated temperature. If the intermediates and end products are obtained as solids, purification can also be carried out by recrystallization or digestion.

If individual compounds of the formula (I) cannot be obtained directly by the routes described above, they can be prepared by derivatization of other compounds of the formula (I) according to the invention.

If the synthesis yields mixtures of isomers, a separation is generally not necessarily required since in some cases the individual isomers can be interconverted during work-up for use or during application (for example under the action of light, acids or bases). Such conversions may also take place after use, for example in the treatment of plants, in the treated plant or in the harmful fungus to be controlled.

In the definitions of the variables given in the formulae above, collective terms are used which are generally representative of the particular substituents or the substituent moieties in composite groups. The term (Cₙ-Cₘ) indicates the number of carbon atoms possible in each case in the substituent or substituent moiety in question:

halogen: fluorine, chlorine, bromine and iodine;

alkyl and the alkyl moieties in composite groups: saturated straight-chain or branched hydrocarbon radicals. The alkyl radicals are preferably (C₁-C₈)-alkyl, in particular (C₁-C₆)-alkyl radicals. According to the invention, it may be preferred to use short-chain alkyl groups, such as (C₁-C₄)-alkyl; on the other hand, it may also be advantageous to employ relatively long-chain alkyl groups, such as (C₅-C₈)-alkyl. Examples of alkyl groups which are preferred according to the invention are methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl.

Haloalkyl: alkyl as defined above, where at least one of the hydrogen atoms or all of the hydrogen atoms in these groups are replaced by halogen atoms as defined above.

In one embodiment, the alkyl groups are substituted at least once or fully by a certain halogen atom, preferably fluorine, chlorine or bromine. In a further embodiment, the alkyl groups are partially or fully halogenated by different halogen atoms; in the case of mixed halogen substitutions, the combination of chlorine and fluorine is preferred.

Examples of preferred haloalkyl radicals which are substituted by one or more halogen atoms of a certain type are (C₁-C₄)-haloalkyl, such as fluoromethyl, chloromethyl, bromomethyl, difluoromethyl, dichloromethyl, trifluoromethyl, trichloromethyl, 1-chloroethyl, 1-bromoethyl, 1-fluoroethyl, 2-fluoroethyl, 2,2-difluoroethyl, 2,2,2-trifluoroethyl, 2,2,2-trichloroethyl, pentafluoroethyl or 1,1,1-trifluoroprop-2-yl.

Examples of preferred mixed substituted haloalkyl radicals are chlorofluoromethyl, dichlorofluoromethyl, chlorodifluoromethyl, 2-chloro-2-fluoroethyl, 2-chloro-2,2-difluoro-ethyl, 2,2-dichloro-2-fluoroethyl.

Alkenyl and alkenyl moieties in composite groups: monounsaturated straight-chain or branched hydrocarbon radicals having a double bond in any position. Preference is given to (C₂-C₈)-alkenyl radicals, more preferably (C₄-C₆)-alkenyl radicals. According to the invention, it may additionally be preferred to use small alkenyl groups, such as (C₂-C₄)-alkenyl, on the other hand, it may also be preferred to use relatively large alkenyl groups, such as (C₅-C₈)-alkenyl.

Examples of preferred alkenyl groups are ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, 1-ethyl-2-methyl-2-propenyl.

Haloalkenyl: alkenyl as defined above, where in these groups at least one of the hydrogen atoms or all of the hydrogen atoms are replaced by halogen atoms as described above under haloalkyl, in particular fluorine, chlorine or bromine.

Alkadienyl: doubly unsaturated straight-chain or branched hydrocarbon radicals having two double bonds in any positions, but not adjacent to one another. Preference is given to (C₄-C₁₀)-alkadienyl radicals, more preferably (C₆-C₈)-alkadienyl radicals.

Examples of preferred alkadienyl radicals are 1,3-butadienyl, 1-methyl-1,3-butadienyl, 2-methyl-1,3-butadienyl, penta-1,3-dien-1-yl, hexa-1,4-dien-1-yl, hexa-1,4-dien-3-yl, hexa-1,4-dien-6-yl, hexa-1,5-dien-1-yl, hexa-1,5-dien-3-yl, hexa-1,5-dien-4-yl, hepta-1,4-dien-1-yl, hepta-1,4-dien-3-yl, hepta-1,4-dien-6-yl, hepta-1,4-dien-7-yl, hepta-1,5-dien-1-yl, hepta-1,5-dien-3-yl, hepta-1,5-dien-4-yl, hepta-1,5-dien-7-yl, hepta-1,6-dien-1-yl, hepta-1,6-dien-3-yl, hepta-1,6-dien-4-yl, hepta-1,6-dien-5-yl, hepta-1,6-dien-2-yl, octa-1,4-dien-1-yl, octa-1,4-dien-2-yl, octa-1,4-dien-3-yl, octa-1,4-dien-6-yl, octa-1,4-dien-7-yl, octa-1,5-dien-1-yl, octa-1,5-dien-3-yl, octa-1,5-dien-4-yl, octa-1,5-dien-7-yl, octa-1,6-dien-1-yl, octa-1,6-dien-3-yl, octa-1,6-dien-4-yl, octa-1,6-dien-5-yl, octa-1,6-dien-2-yl, deca-1,4-dienyl, deca-1,5-dienyl, deca-1,6-dienyl, deca-1,7-dienyl, deca-1,8-dienyl, deca-2,5-dienyl, deca-2,6-dienyl, deca-2,7-dienyl, deca-2,8-dienyl and the like.

Haloalkadienyl: alkadienyl as defined above, where in these groups at least one of the hydrogen atoms or all of the hydrogen atoms are replaced by halogen atoms as described above under haloalkyl, in particular fluorine, chlorine or bromine.

Alkynyl and the alkynyl moieties in composite groups: straight-chain or branched hydrocarbon radicals having one or two triple bonds in any positions except for adjacent positions. Preference is given to (C₂-C₈)-alkynyl radicals, more preferably (C₄-C₆)-alkynyl radicals.

Preferred alkynyl radicals are: ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, 1-ethyl-1-methyl-2-propynyl.

Haloalkynyl: alkynyl as defined above, where in these groups at least one of the hydrogen atoms or all of the hydrogen atoms are replaced by halogen atoms as described above under haloalkyl, in particular fluorine, chlorine or bromine.

Cycloalkyl and the cycloalkyl moieties in composite groups: monocyclic saturated hydrocarbon groups. Preference is given to (C₃-C₈)-cycloalkyl radicals, more preferably (C₄-C₆)-cycloalkyl radicals.

Examples of preferred cycloalkyl radicals are cyclopropyl, cyclobutyl, cyclopentyl, cyclohexyl, cycloheptyl and cyclooctyl.

Halocycloalkyl: cycloalkyl as defined above, where in these groups at least one of the hydrogen atoms or all of the hydrogen atoms are replaced by halogen atoms as described above under haloalkyl, in particular fluorine, chlorine or bromine.

Cycloalkenyl and the cycloalkenyl moieties in composite groups: monocyclic monounsaturated hydrocarbon radicals having a double bond in any position. Preference is given to (C₃-C₈)-cycloalkenyl, more preferably (C₅-C₆)-cycloalkenyl.

Examples of preferred cycloalkenyl radicals are cyclopenten-1-yl, cyclopenten-3-yl, cyclohexen-1-yl, cyclohexen-3-yl, cyclohexen-4-yl.

Halocycloalkenyl: cycloalkenyl as defined above, where in these groups at least one of the hydrogen atoms or all of the hydrogen atoms are replaced by halogen atoms as described above under haloalkyl, in particular fluorine, chlorine or bromine.

Bicycloalkyl: a bicyclic hydrocarbon radical, (C₅-C₁₀)-bicycloalkyl being preferred. Further preferred are (C₇-C₈)-bicycloalkyl radicals.

Examples of preferred bicycloalkyl radicals are bicyclo[2.2.1]hept-1-yl, bicyclo[2.2.1]-hept-2-yl, bicyclo[2.2.1]hept-7-yl, bicyclo[2.2.2]oct-1-yl, bicyclo[2.2.2]oct-2-yl, bicyclo[3.3. O]octyl, bicyclo[4.4.0]decyl.

Halobicycloalkyl: bicycloalkyl as defined above, where in these groups at least one of the hydrogen atoms or all of the hydrogen atoms are replaced by halogen atoms as described above under haloalkyl, in particular fluorine, chlorine or bromine.

Alkoxy: an alkyl group as defined above which is attached via an oxygen atom. Preference is given to (C₁-C₈)-alkoxy radicals, further preferred are (C₂-C₆)-alkoxy radicals. According to the invention, it may be preferred to use small alkoxy groups, such as (C₁-C₄)-alkoxy, on the other hand, it may also be preferred to use relatively large alkoxy groups, such as (C₅-C₈)-alkoxy.

Examples of preferred alkoxy radicals are: methoxy, ethoxy, n-propoxy, 1-methyl-ethoxy, butoxy, 1-methylpropoxy, 2-methylpropoxy or 1,1-dimethylethoxy, pentoxy, 1-methylbutoxy, 2-methylbutoxy, 3-methylbutoxy, 1,1-dimethylpropoxy, 1,2-dimethyl-propoxy, 2,2-dimethylpropoxy, 1-ethylpropoxy, hexoxy, 1-methylpentoxy, 2-methyl-pentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy or 1-ethyl-2-methylpropoxy.

Haloalkoxy: alkoxy as defined above, where in these groups at least one of the hydrogen atoms or all of the hydrogen atoms are replaced by halogen atoms as described above under haloalkyl, in particular fluorine, chlorine or bromine.

According to the invention, it may be preferred to use short-chain haloalkoxy groups, such as (C₁-C₄)-haloalkoxy, on the other hand, it may also be preferred to use relatively long-chain haloalkoxy groups, such as (C₅-C₈)-haloalkoxy.

Examples of preferred short-chain haloalkoxy radicals are OCH₂F, OCHF₂, OCF₃, OCH₂Cl, OCHCl₂, OCCl₃, chlorofluoromethoxy, dichlorofluoromethoxy, chlorodifluoro-methoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, OC₂F₅, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, OCH₂—C₂F₅, OCF₂—C₂F₅, 1-(CH₂F)-2-fluoroethoxy, 1-(CH₂Cl)-2-chloroethoxy, 1-(CH₂Br)-2-bromoethoxy, 4-fluorobutoxy, 4-chlorobutoxy, 4-bromobutoxy or nonafluorobutoxy.

Examples of preferred relatively long-chain haloalkoxy radicals are 5-fluoropentoxy, 5-chloropentoxy, 5-bromopentoxy, 5-iodopentoxy, undecafluoropentoxy, 6-fluorohexoxy, 6-chlorohexoxy, 6-bromohexoxy, 6-iodohexoxy or dodecafluorohexoxy.

Alkenyloxy: alkenyl as defined above which is attached via an oxygen atom. Preferred is (C₂-C₈)-alkenyloxy, more preferably (C₃-C₆)-alkenyloxy. According to the invention, it may be preferred to use short-chain alkenyloxy radicals, such as (C₂-C₄)-alkenyloxy, on the other hand, it may also be preferred to use relatively long-chain alkenyloxy groups, such as (C₅-C₈)-alkenyloxy.

Examples of preferred alkenyloxy radicals are 1-propenyloxy, 2-propenyloxy, 1-methyl-ethenyloxy, 1-butenyloxy, 2-butenyloxy, 3-butenyloxy, 1-methyl-1-propenyloxy, 2-methyl-1-propenyloxy, 1-methyl-2-propenyloxy, 2-methyl-2-propenyloxy, 1-pentenyloxy, 2-pentenyloxy, 3-pentenyloxy, 4-pentenyloxy, 1-methyl-1-butenyloxy, 2-methyl-1-butenyloxy, 3-methyl-1-butenyloxy, 1-methyl-2-butenyloxy, 2-methyl-2-butenyloxy, 3-methyl-2-butenyloxy, 1-methyl-3-butenyloxy, 2-methyl-3-butenyloxy, 3-methyl-3-butenyl, 1,1-dimethyl-2-propenyloxy, 1,2-dimethyl-1-propenyloxy, 1,2-dimethyl-2-propenyloxy, 1-ethyl-1-propenyloxy, 1-ethyl-2-propenyloxy, 1-hexenyloxy, 2-hexenyl-oxy, 3-hexenyloxy, 4-hexenyloxy, 5-hexenyloxy, 1-methyl-1-pentenyloxy, 2-methyl-1-pentenyloxy, 3-methyl-1-pentenyloxy, 4-methyl-1-pentenyloxy, 1-methyl-2-pentenyloxy, 2-methyl-2-pentenyloxy, 3-methyl-2-pentenyloxy, 4-methyl-2-pentenyloxy, 1-methyl-3-pentenyloxy, 2-methyl-3-pentenyloxy, 3-methyl-3-pentenyloxy, 4-methyl-3-pentenyloxy, 1-methyl-4-pentenyloxy, 2-methyl-4-pentenyloxy, 3-methyl-4-pentenyloxy, 4-methyl-4-pentenyloxy, 1,1-dimethyl-2-butenyloxy, 1,1-dimethyl-3-butenyloxy, 1,2-dimethyl-1-butenyloxy, 1,2-dimethyl-2-butenyloxy, 1,2-dimethyl-3-butenyloxy, 1,3-dimethyl-1-butenyloxy, 1,3-dimethyl-2-butenyloxy, 1,3-dimethyl-3-butenyloxy, 2,2-dimethyl-3-butenyloxy, 2,3-dimethyl-1-butenyloxy, 2,3-dimethyl-2-butenyloxy, 2,3-dimethyl-3-butenyloxy, 3,3-dimethyl-1-butenyloxy, 3,3-dimethyl-2-butenyloxy, 1-ethyl-1-butenyloxy, 1-ethyl-2-butenyloxy, 1-ethyl-3-butenyloxy, 2-ethyl-1-butenyloxy, 2-ethyl-2-butenyloxy, 2-ethyl-3-butenyloxy, 1,1,2-trimethyl-2-propenyloxy, 1-ethyl-1-methyl-2-propenyloxy, 1-ethyl-2-methyl-1-propenyloxy and 1-ethyl-2-methyl-2-propenyloxy.

Haloalkenyloxy: alkenyloxy as defined above, where in these groups at least one of the hydrogen atoms or all of the hydrogen atoms are replaced by halogen atoms as described above under haloalkyl, in particular fluorine, chlorine or bromine.

Alkynyloxy: alkynyl as mentioned above which is attached via an oxygen atom. Preferred is (C₂-C₈)-alkynyloxy, more preferably (C₃-C₆)-alkynyloxy. According to the invention, it may be preferred to use short-chain alkynyloxy radicals, such as (C₂-C₄)-alkynyloxy, on the other hand, it may also be preferred to use relatively long-chain alkynyloxy groups, such as (C₅-C₈)-alkynyloxy.

Examples of preferred alkynyloxy radicals are 2-propynyloxy, 2-butynyloxy, 3-butynyloxy, 1-methyl-2-propynyloxy, 2-pentynyloxy, 3-pentynyloxy, 4-pentynyloxy, 1-methyl-2-butynyloxy, 1-methyl-3-butynyloxy, 2-methyl-3-butynyloxy, 1-ethyl-2-propynyloxy, 2-hexynyloxy, 3-hexynyloxy, 4-hexynyloxy, 5-hexynyloxy, 1-methyl-2-pentynyloxy, 1-methyl-3-pentynyloxy.

Haloalkynyloxy: alkynyloxy as defined above, where in these groups at least one of the hydrogen atoms or all of the hydrogen atoms are replaced by halogen atoms as described above under haloalkyl, in particular fluorine, chlorine or bromine.

Alkylene: divalent unbranched chains of CH₂ groups. Preference is given to (C₁-C₆)-alkylene, more preference to (C₂-C₄)-alkylene; furthermore, it may be preferred to use (C₁-C₃)-alkylene groups. Examples of preferred alkylene radicals are CH₂, CH₂CH₂, CH₂CH₂CH₂, CH₂(CH₂)₂CH₂, CH₂(CH₂)₃CH₂ and CH₂(CH₂)₂—CH₂.

Oxyalkylene: alkylene as defined above, where one valency is attached to the skeleton via an oxygen atom. Examples of preferred oxyalkylene radicals are OCH₂, OCH₂CH₂, OCH₂CH₂CH₂ and OCH₂(CH₂)₂CH₂.

Oxyalkyleneoxy: alkylene as defined above, where both valencies are attached to the skeleton via an oxygen atom. Examples of preferred oxyalkyleneoxy radicals are OCH₂O, OCH₂CH₂O and OCH₂CH₂CH₂O.

Alkylthio: alkyl as defined above which is attached via an S atom.

Alkylsulfinyl: alkyl as defined above which is attached via an SO group.

Alkylsulfonyl: alkyl as defined above which is attached via an S(O)₂ group.

Aryl: an aromatic hydrocarbon radical, (C₆-C₁₄)-aryl radicals being preferred and (C₆-C₁₀)-aryl radicals being particularly preferred. Examples of preferred aryl radicals are phenyl, naphthyl and anthryl.

The aryl radicals may be substituted by at least one halogen atom or fully by halogen atoms as defined above. According to the invention, it may be advantageous to employ haloaryl groups, where aryl is as defined above. Particularly preferred may be halophenyl and halonaphthyl.

Aryloxy: aryl as defined above, where the aryl radical is attached to the skeleton via an oxygen atom.

Arylthio: aryl as defined above, where the aryl radical is attached to the skeleton via a sulfur atom.

A five- to ten-membered saturated, partially unsaturated or aromatic heterocycle which contains one, two, three or four heteroatoms from the group consisting of O, N and S: a five-, six-, seven-, eight-, nine- or ten-membered saturated, partially unsaturated or aromatic heterocycle. Preferably, the heterocycle is a five- or six-membered saturated, partially unsaturated or aromatic heterocycle which contains one, two, three or four heteroatoms from the group consisting of O, N and S, as defined below. The heterocycle in question may be attached via a carbon atom or via a nitrogen atom, if present. According to the invention, it may be preferred that the heterocycle in question is attached via carbon, on the other hand, it may also be preferred that the heterocycle is attached via nitrogen.

Examples of five- to ten-membered heterocycles are:
seven-membered saturated or partially unsaturated heterocycles containing one, two, three or four heteroatoms from the group consisting of oxygen, nitrogen and sulfur as ring members: for example mono- and bicyclic heterocycles having 7 ring members and containing, in addition to carbon ring members, one to three nitrogen atoms and/or one oxygen or sulfur atom or one or two oxygen and/or sulfur atoms, for example tetra- and hexahydroazepinyl, such as 2,3,4,5-tetrahydro[1H]azepin-1-, -2-, -3-, -4-, -5-, -6- or -7-yl, 3,4,5,6-tetrahydro[2H]-azepin-2-, -3-, -4-, -5-, -6- or -7-yl, 2,3,4,7-tetrahydro[1H]azepin-1-, -2-, -3-, -4-, -5-, -6- or -7-yl, 2,3,6,7-tetrahydro[1H]azepin-1-, -2-, -3-, -4-, -5-, -6- or -7-yl, hexahydroazepin-1-, -2-, -3- or -4-yl, tetra- and hexahydrooxepinyl, such as 2,3,4,5-tetrahydro[1H]oxepin-2-, -3-, -4-, -5-, -6- or -7-yl, 2,3,4,7-tetrahydro[1H]oxepin-2-, -3-, -4-, -5-, -6- or -7-yl, 2,3,6,7-tetrahydro[1H]oxepin-2-, -3-, -4-, -5-, -6- or -7-yl, hexahydroazepin-1-, -2-, -3- or -4-yl, tetra- and hexahydro-1,3-diazepinyl, tetra- and hexahydro-1,4-diazepinyl, tetra- and hexahydro-1,3-oxazepinyl, tetra- and hexahydro-1,4-oxazepinyl, tetra- and hexahydro-1,3-dioxepinyl, tetra- and hexahydro-1,4-dioxepinyl and the corresponding ylidene radicals.

A five- or six-membered saturated, partially unsaturated or aromatic heterocycle which contains one to four heteroatoms from the group consisting of O, N and S, where the heterocycle in question may be attached via C or N:
a five- or six-membered saturated or partially unsaturated heterocycle (hereinbelow also referred to as heterocyclyl) which contains one, two, three or four heteroatoms from the group consisting of oxygen, nitrogen and sulfur as ring members: for example monocyclic saturated or partially unsaturated heterocycles containing, in addition to carbon ring members, one to three nitrogen atoms and/or one oxygen or sulfur atom or one or two oxygen and/or sulfur atoms, for example 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-50 isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-di-hydropyrazol-1-yl, 2,3-dihydropyrazol-2-yl, 2,3-dihydropyrazol-3-yl, 2,3-dihydro-pyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4, 5-55 dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydro-oxazol-3-yl, 3,4-dihydrooxazol-4-yl, 2-piperidinyl, 3-piperidinyl, 4-piperidinyl, 1, 3-60 dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexa-hydropyrimidinyl, 5-hexahydropyrimidinyl, 2-piperazinyl, 1,3,5-hexahydrotriazin-2-yl and 1,2,4-hexahydrotriazin-3-yl and the corresponding ylidene radicals;
a five-membered aromatic heterocycle which contains one, two, three or four heteroatoms from the group consisting of oxygen, nitrogen and sulfur, where the aromatic heterocycle may be attached via carbon or via nitrogen. According to the invention, it may be preferred that the aromatic heterocycle in question is attached via carbon, on the other hand, it may also be preferred that the aromatic heterocycle is attached via nitrogen:
5-membered heteroaryl which contains one, two, three or four nitrogen atoms or one, two or three nitrogen atoms and/or one sulfur or oxygen atom: 5-membered heteroaryl groups which, in addition to carbon atoms, may contain one to four nitrogen atoms or one to three nitrogen atoms and/or one sulfur or oxygen atom as ring members, for example furyl, thienyl, pyrrolyl, pyrazolyl, imidazolyl, triazolyl (1,2,3-; 1,2,4-triazolyl), tetrazolyl, oxazolyl, isoxazolyl, 1,3,4-oxadiazolyl, thiazolyl, isothiazolyl and thiadiazolyl, in particular 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1-tetrazolyl, 5-tetrazolyl, 1,3,4-thiadiazol-2-yl and 1,3,4-triazol-2-yl.
According to the invention, 5-membered heteroaryl groups may be preferred which are attached via carbon and which contain one to four, one to three or one or two nitrogen atoms and one sulfur or oxygen atom as ring members. Examples of these are: 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl and 1,3,4-triazol-2-yl, 5-tetrazolyl.
Furthermore, five-membered heteroaryl groups which are attached via carbon and which contain an oxygen atom or a sulfur atom may be advantageous, such as, for example, 2-furyl, 3-furyl, 2-thienyl, 3-thienyl.
On the other hand, according to the invention preference may also be given to 5-membered heteroaryl groups which are attached via nitrogen and which contain, as ring members, one to three nitrogen atoms, such as, for example, pyrrol-1-yl, pyrazol-1-yl, imidazol-1-yl, 1,2,3-triazol-1-yl and 1,2,4-triazol-1-yl;
a six-membered aromatic heterocycle which contains one, two, three or four heteroatoms from the group consisting of oxygen, nitrogen and sulfur, where the aromatic heterocycle may be attached via carbon or via nitrogen. According to the invention, it may be preferred that the aromatic heterocycle is attached via carbon, on the other hand, it may also be preferred that the aromatic heterocycle is attached via nitrogen. Examples are:
a six-membered aromatic heterocycle which contains one, two or three or one, two, three or four nitrogen atoms: 6-membered heteroaryl groups which, in addition to carbon atoms, may contain one to three or one to four nitrogen atoms as ring members, for example pyridinyl, pyrimidinyl, pyrazinyl, pyridazinyl, 1,2,3-triazinyl, 1,2,4-triazinyl, 1,3,5-triazinyl, in particular 2-pyridinyl, 3-pyridinyl, 4-pyridinyl, 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl.

Heteroaryloxy: heteroaryl as defined above where the heteroaryl radical is attached to the skeleton via an oxygen atom.

Heteroarylthio: heteroaryl as defined above where the heteroaryl radical is attached to the skeleton via a sulfur atom.

The scope of the present invention embraces the (R) and (S) isomers or rotamers and the racemates of compounds of the formula (I) having chiral centers. The compounds according to the invention and/or their salts may be present in various crystal modifications which may differ from one another in their biological activity. They are likewise provided by the present invention.

With a view to the intended use of the triazolopyrimidines of the formula (I), particular preference is given to the following meanings of the substituents, in each case on their own or in combination. The preferred substituents or preferred combinations of substituents apply correspondingly to the precursors of the compounds of the formula (I):

In the 6-position, the compounds of the present invention contain an optionally substituted five-membered aromatic heterocycle which contains one, two, three or four heteroatoms from the group consisting of O, S and N and which may be attached to the triazolopyrimidine skeleton via a ring carbon atom or via a ring nitrogen atom. Het groups which are preferred according to the invention contain one to four nitrogen atoms or one to three nitrogen atoms and/or one sulfur or oxygen atom.

According to the invention, preference may be given to 5-membered heteroaryl groups which are attached via carbon and which contain, as ring members, one to four nitrogen atoms, one to three or one or two nitrogen atoms and/or one sulfur or oxygen atom.

On the other hand, preference according to the invention may also be given to Het which are 5-membered heteroaryl groups which are attached via nitrogen and which contain, as ring members, one to four, one to three or one or two nitrogen atoms.

According to one embodiment of the present invention, preference is given to furyl and thienyl, in particular 2-furyl, 3-furyl, 2-thienyl and 3-thienyl.

According to a further embodiment of the present invention, preferred Het are heteroaryl groups which, as heteroatoms, contain at least one nitrogen atom, preferably exactly one nitrogen atom, and also one sulfur or oxygen atom. Examples are oxazolyl, isoxazolyl, oxadiazolyl, thiazolyl, isothiazolyl and thiadiazolyl, in particular 2-oxazolyl, 4-oxazolyl, 5-oxazolyl, 2-thiazolyl, 4-thiazolyl, 5-thiazolyl, 3-isoxazolyl, 4-isoxazolyl, 5-isoxazolyl, 3-isothiazolyl, 4-isothiazolyl, 5-isothiazolyl, 1,2,4-oxadiazol-3-yl, 1,2,4-oxadiazol-5-yl, 1,2,4-thiadiazol-3-yl and 1,2,4-thiadiazol-5-yl.

According to another embodiment of the present invention, preferred Het are heteroaryl groups which, in addition to carbon atoms, contain only nitrogen atoms as heteroatoms. According to this embodiment, Het is preferably pyrrolyl, pyrazolyl, imidazolyl, triazolyl (1,2,3-; 1,2,4-triazolyl) or tetrazolyl, particularly preferably 2-pyrrolyl, 3-pyrrolyl, 3-pyrazolyl, 4-pyrazolyl, 5-pyrazolyl, 2-imidazolyl, 4-imidazolyl, tetrazol-1-yl or tetrazol-5-yl.

According to the invention, preferred Het may be 5-membered heteroaryl groups which are attached via carbon and which contain one to three nitrogen atoms or one or two nitrogen atoms and one sulfur or oxygen atom as ring members. Examples of these are: 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-thiadiazol-3-yl, 1,2,4-thiadiazol-5-yl, 1,2,4-triazol-3-yl, 1,3,4-thiadiazol-2-yl and 1,3,4-triazol-2-yl.

On the other hand, according to the invention preference may also be given to Het which are 5-membered heteroaryl groups which are attached via nitrogen and which contain, as ring members, one to three nitrogen atoms, such as, for example, pyrrol-1-yl, pyrazol-1-yl, imidazol-1-yl, 1,2,3-triazol-1-yl and 1,2,4-triazol-1-yl.

According to a further embodiment of the invention, Het is a 5-membered heteroaryl group which is optionally substituted by one or two L, which contains two nitrogen atoms as ring members and which is selected from the group consisting of 1,2,3-thiadiazolyl, 1,2,4-thiadiazolyl, 1,3,4-thiadiazolyl, 1,2,5-thiadiazolyl, 1,2,4-oxadiazolyl, 1,3,4-oxadiazolyl and 1,2,5-oxadiazolyl. In a preferred form of this embodiment, Het is unsubstituted. In a further preferred form, Het is substituted by a substituent L. In the compounds of this embodiment, Het may preferably contain one or two identical or different substituents L, preferably identical substituents L, L being defined as above. If Het contains two substituents L, Het is present as in agriculturally acceptable salt, as described above. Particularly preferably, Het contains one substituent L. Het may be attached to the triazolopyrimidine skeleton via a ring carbon or via a ring nitrogen, preferably via a carbon atom.

Particularly preferred Het are optionally substituted 1,2,3-thiadiazolyl, 1,3,4-thiadiazolyl and 1,3,4-oxadiazolyl. Particular preference is given to 1,2,3-thiadiazol-4-yl, 1,2,3-thiadiazol-5-yl, 1,3,4-thiadiazol-2-yl and 1,3,4-oxadiazol-2-yl, which may be substituted by one or two substituents L.

According to a further form, Het is optionally, as defined above or below, substituted 1,2,3-thiadiazolyl, 1,2,4-thiadiazolyl, 1,3,4-thiadiazolyl or 1,2,5-thiadiazolyl. Here, Het is particularly preferably 5-substituted 1,2,3-thiadiazol-4-yl, 4-substituted 1,2,3-thiadiazol-5-yl, 3-substituted 1,2,4-thiadiazol-5-yl, 5-substituted 1,2,4-thiadiazol-3-yl, 2-substituted 1,3,4-thiadiazol-5-yl or 3-substituted 1,2,5-thiadiazol-4-yl.

In a further form, Het is optionally, as defined above or below, substituted 1,2,4-oxadiazolyl, 1,3,4-oxadiazolyl or 1,2,5-oxadiazolyl.

In the compounds of the present invention, Het may contain one to four or one to three or one or two identical or different substituents L, preferably identical substituents L. With particular preference, Het contains one or two substituents L, particularly preferably one or two identical substituents L. Furthermore preferably, Het has two identical substituents L.

According to the invention, it may be generally preferred that Het contains at least one substituent L which is preferably located in the position ortho to the point of attachment to the pyrimidine skeleton.

Examples of preferred substitution patterns of Het embraced by the invention are as stated in Table A below, where L¹, L² and L³ within a heteroaromatic radical Het are each identical or different L:

TABLE A

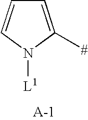

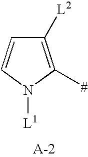

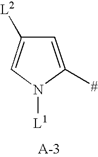

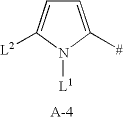

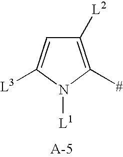

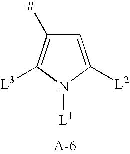

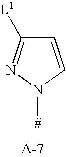

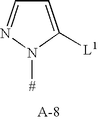

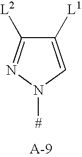

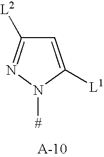

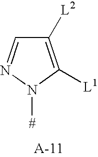

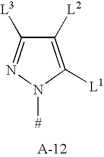

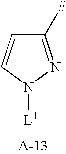

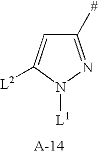

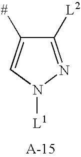

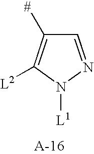

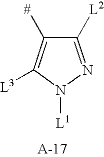

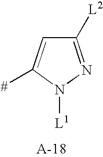

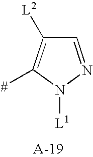

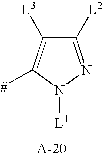

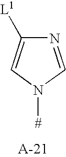

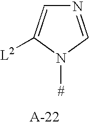

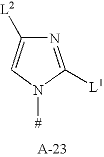

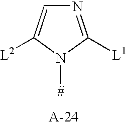

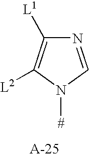

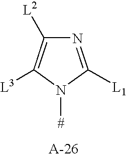

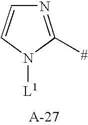

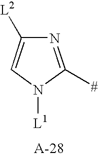

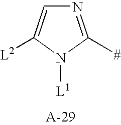

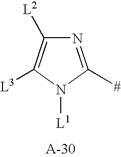

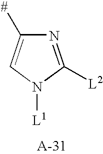

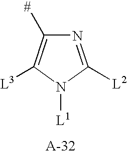

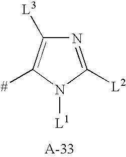

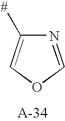

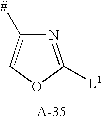

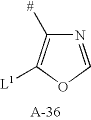

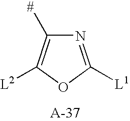

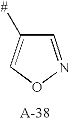

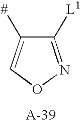

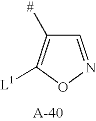

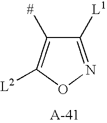

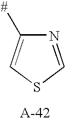

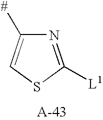

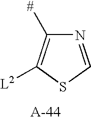

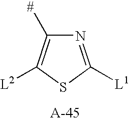

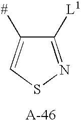

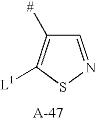

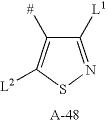

In the structures in Table A, # denotes in each case the point of attachment of the Het in question to the triazolopyrimidine skeleton of the compounds according to the invention or the precursors thereof.

If Het is pyrrolyl, particular preference is given to a substitution pattern selected from the group consisting of A-1, A-2, A-3, A-4 and A-5, in particular selected from the group consisting of A-2 and A-4 according to Table A.

If Het is pyrazolyl, a substitution pattern is selected from the group consisting of A-7, A-8, A-10, A-11, A-13, A-14, A-15, A-16 and A-19. Particular preference is given to A-10, in particular where L¹=L².

If Het is imidazolyl, a substitution pattern is selected from the group consisting of A-21, A-22, A-23, A-24, A-25, A-26, A-27, A-28, A-29 and A-30, in particular selected from the group consisting of A-22, A-23, A-24, A-25 and A-27, A-28, and A-29.

If Het is oxazolyl, particular preference is given to a substitution pattern selected from the group consisting of A-36 and A-37.

If Het is isoxazolyl, particular preference is given to a substitution pattern selected from the group consisting of A-39, A-40 and A-41.

If Het is thiazolyl, particular preference is given to a substitution pattern selected from the group consisting of A-44 and A-45.

If Het is tetrazolyl, it is tetrazol-1-yl, tetrazol-2-yl and tetrazol-5-yl. In the compounds of the present invention, the tetrazolyl radical may preferably contain one or two identical or different substituents L, preferably identical substituents L, where L is as defined above. Particularly preferably, Het contains a substituent L. With particular preference, Het is here 5-methyltetrazol-1-yl, 5-methyltetrazol-2-yl, 5-chlorotetrazol-1-yl, 5-chlorotetrazol-2-yl, 5-bromotetrazol-1-yl, 5-bromotetrazol-2-yl, 1-methyltetrazol-5-yl or 2-methyltetrazol-5-yl.

In a further preferred embodiment, Het is thiadiazol, preferably thiadiazol-2-yl, which may be unsubstituted or substituted by L, preferably in position 5.

According to the present invention, L is in each case particularly preferably selected from the group consisting of halogen, cyano, nitro, (C₁-C₄)-alkyl, (C₃-C₆)-cycloalkyl, (C₁-C₄)-cycloalkoxy, —COO(C₁-C₄), —CONH₂ or —CSNH₂; with particular preference, L is methyl, ethyl, isopropyl, cyclopropyl, fluorine, chlorine, bromine, iodine, —COOCH₃ or CN. In further preferred compounds of the present invention, Het has one, two or three identical or different substituents L selected from the group consisting of halogen, cyano, nitro, amino, (C₁-C₆)-alkylamino, di-(C₁-C₆)-alkylamino, (C₁-C₆)-alkyl, (C₁-C₆)-haloalkyl, (C₁-C₆)-alkoxy, (C₁-C₆)-haloalkoxy, NH(CO)—(C₁-C₆)-alkyl, C(S)A² and C(O)A², where A² is as defined above and is preferably (C₁-C₄)-alkoxy, NH₂, (C₁-C₄)-alkylamino or di-(C₁-C₄)-alkylamino.

Particularly preferred L are selected from the group consisting of fluorine, chlorine, bromine, cyano, nitro, (C₁-C₄)-alkyl, (C₁-C₄)-haloalkyl, (C₁-C₄)-alkoxy and (C₁-C₄)-alkylcarbonyl, with particular preference from the group consisting of fluorine, chlorine, (C₁-C₂)-alkyl, such as methyl or ethyl, (C₁-C₂)-fluoroalkyl, such as trifluoroalkyl, (C₁-C₂)-alkoxy, such as methoxy, or (C₁-C₂)-alkoxycarbonyl, such as methoxycarbonyl.

According to a preferred embodiment of the present invention, Het has at least one substituent located in the position ortho to the point of attachment to the skeleton to which Het is attached. The L located in the ortho position is in particular fluorine, chlorine, (C₁-C₂)-alkyl, such as methyl or ethyl, (C₁-C₂)-fluoroalkyl, such as trifluoroalkyl, or (C₁-C₂)-alkoxy, such as methoxy.

In the compounds according to the invention or their precursors, if L is attached to a ring nitrogen of Het, L is in each case independently with particular preference:

C(═O)A², C(═S)A²; or

(C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₃-C₈)-cycloalkyl, (C₃-C₈)-halocycloalkyl, (C₃-C₈)-cycloalkenyl, (C₃-C₈)-halocycloalkenyl, (C₁-C₈)-alkoximinoalkyl, (C₂-C₈)-alkenyloximino-(C₁-C₈)-alkyl or (C₂-C₈)-alkynyloximino-(C₁-C₈)-alkyl. Particularly preferably, L is (C₁-C₆)-alkyl or (C₁-C₆)-haloalkyl, more preferably (C₁-C₄)-alkyl or (C₁-C₄)-haloalkyl, in particular methyl or ethyl, particularly preferably methyl.

If L is attached to a ring carbon of Het, L is in each case independently preferably:

C(═O)A², C(═S)A²; or

(C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₃-C₈)-cycloalkyl, (C₃-C₈)-halocycloalkyl, (C₃-C₈)-cycloalkenyl, (C₃-C₈)-halocycloalkenyl, (C₁-C₈)-alkoximinoalkyl, (C₂-C₈)-alkenyloximino-(C₁-C₈)-alkyl, (C₂-C₈)-alkynyloximino-(C₁-C₈)-alkyl; or

halogen, cyano, hydroxyl, nitro, NR⁵R⁶, NR⁵—(C═O)—R⁶, S(═O)ₙA¹; or

(C₁-C₈)-alkoxy, (C₁-C₈)-haloalkoxy, (C₁-C₈)-alkenyloxy, (C₁-C₈)-haloalkenyloxy, (C₁-C₈)-alkynyloxy, (C₁-C₈)-haloalkynyloxy, (C₁-C₈)-cycloalkoxy, (C₁-C₈)-halocycloalkoxy; where
A¹ is amino, (C₁-C₆)-alkyl, (C₁-C₆)-haloalkyl, (C₁-C₆)-alkylamino or di-(C₁-C₆)-alkylamino;
n is 0, 1 or 2;
A² is one of the groups mentioned under A¹ or (C₂-C₈)-alkenyl, (C₂-C₈)-haloalkenyl, (C₂-C₈)-alkynyl, (C₂-C₈)-haloalkynyl, (C₁-C₆)-alkoxy, (C₁-C₆)-haloalkoxy, (C₂-C₈)-alkenyloxy, (C₂-C₈)-haloalkenyloxy, (C₂-C₈)-alkynyloxy, (C₂-C₈)-haloalkynyloxy, (C₃-C₈)-cycloalkoxy or (C₃-C₈)-halocycloalkoxy;
R⁵, R⁶ independently of one another are hydrogen or (C₁-C₆)-alkyl, (C₁-C₆)-haloalkyl, (C₂-C₈)-haloalkenyl, (C₂-C₈)-alkynyl, (C₂-C₈)-haloalkynyl, (C₃-C₈)-cycloalkyl, (C₃-C₈)-halocycloalkyl, (C₃-C₈)-cycloalkenyl or (C₃-C₈)-halocycloalkenyl.

Here, L is, when it is attached to a ring nitrogen of Het, in each case particularly preferably (C₁-C₄)-alkyl, (C₃-C₆)-cycloalkyl, —COO(C₁-C₄), —CONH₂ or —CSNH₂, in particular methyl, ethyl, isopropyl, cyclopropyl or —COOCH₃.

In particular for the fungicidal action of the compounds according to the invention, it may be preferable if R is hydrogen. In one embodiment of the present invention, R² is hydrogen and R¹ is different from hydrogen. Furthermore, it may be preferable for at least one of R¹ and R² to be different from hydrogen. Likewise, preference is given to compounds of the formula (I) in which R¹ and R² are different from hydrogen. Among these, compounds of the formula (I) in which R² is (C₁-C₄)-alkyl, especially methyl or ethyl, are preferred.

In a further embodiment of the invention, R¹ and R² are both hydrogen.

For the fungicidal action of the compounds according to the invention it is furthermore advantageous if the substituents R¹, X and Y independently of one another and preferably in combination particularly preferably have the meanings given below.

In preferred compounds of the formula (I) according to the invention, R¹ is straight-chain or branched unsubstituted or substituted (C₁-C₈)-alkyl, (C₁-C₈)-haloalkyl, (C₂-C₈)-alkenyl, (C₂-C₈)-alkynyl, (C₃-C₈)-cycloalkyl, unsubstituted or substituted phenyl or naphthyl or a five- or six-membered saturated, partially unsaturated or aromatic heterocycle which contains one, two, three or four heteroatoms from the group consisting of O, N and S.

R¹ is in particular (C₁-C₆)-alkyl, (C₂-C₆)-alkenyl, (C₂-C₆)-alkynyl, (C₃-C₆)-cycloalkyl, where these radicals may be substituted 1, 2, 3, 4 or 5 times by halogen, (C₁-C₆)-alkyl or (C₁-C₆)-haloalkyl.

Among these a particularly preferred embodiment relates to compounds of the formula (I) in which R is a group B:

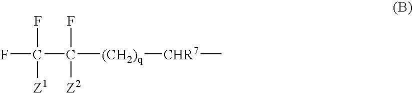

in which
Z¹ is hydrogen, fluorine or (C₁-C₄)-fluoroalkyl, Z² is hydrogen or fluorine or
Z¹ and Z² together form a double bond;
q is 0 or 1; and
R⁷ is hydrogen or methyl.

In a further embodiment, particular preference is moreover given to compounds of the formula (I) in which R¹ is (C₃-C₆)-cycloalkyl which may be substituted by (C₁-C₄)-alkyl.

Likewise preferred are compounds of the formula (I) in which R¹ and R² together with the nitrogen atom to which they are attached are saturated or monounsaturated, in particular 5- or 6-membered heterocyclyl as defined above. Among these, preference is given to those compounds in which R¹ and R² together with the nitrogen atom to which they are attached form an optionally substituted piperidinyl, morpholinyl or thiomorpholinyl ring, especially a piperidinyl ring. Heterocyclyl is in particular unsubstituted or substituted by 1, 2 or 3 substituents R^{a}, preferred substituents R^{a} on heterocyclyl being selected from the group consisting of halogen, (C₁-C₄)-alkyl and (C₁—Cl)-haloalkyl. Among these, preference is given in particular to compounds (I) in which R¹ and R² together with the nitrogen atom, to which they are attached, form a 4-methylpiperidine ring, a 4-trifluoromethylpiperidine ring, a morpholine ring or a 3,4-dimethylpiperidine ring and especially a 4-methylpiperidine ring or a 3,4-dimethylpiperidine ring.

The invention furthermore particularly preferably provides compounds (I) in which R¹ and R² together with the nitrogen atom to which they are attached are 5- or 6-membered heteroaryl as defined above which may be substituted or unsubstituted, preferably by 1, 2 or 3 groups R^{a}. In this case, the group NR¹R² forms in particular a pyrazole ring which, if appropriate, is substituted in the manner described above and especially by 1 or 2 of the following radicals: halogen, (C₁-C₄)-alkyl or (C₁-C₄)-haloalkyl, in particular by 2 methyl groups or two trifluoromethyl groups in the 3,5-position.

Very particular preference is given to compounds of the formula (I) in which R¹ is selected from the group consisting of: methyl, ethyl, CH(CH₃)CH₂CH₃, CH₂CH(CH₃)₂, CH(CH₃)CH(CH₃)₂, CH(CH₃)C(CH₃)₃, CH(CH₃)CF₃, CH(CH₃)CF₃, CH(CH₃)CCl₃, CH₂CF₂CF₃, CH₂C(CH₃)═CH₂, CH₂CH═CH₂, cyclopentyl, cyclohexyl, benzyl; and R² is hydrogen or methyl; and also to compounds (I) in which R¹ and R² together are —(CH₂)₂CH(CH₃)(CH₂)₂—, —(CH₂)₂CH(CF₃)(CH₂)₂— or —(CH₂)₂—O—(CH₂)₂—.

In the compounds of the formula (I) according to the invention and the corresponding intermediates, X has the meanings given further above. Preferably, X is (C₁-C₄)-alkyl, more preferably (C₁-C₂)-alkyl, thus methyl or ethyl, (C₁-C₄)-haloalkyl, such as, for example, fluoromethyl, chloromethyl, bromomethyl, difluoromethyl, dichloromethyl, trifluoromethyl, trichloromethyl, chlorofluoromethyl, dichlorofluoromethyl or chlorodifluoromethyl. Furthermore preferably, X is (C₂-C₆)-alkenyl, or (C₂-C₆)-haloalkenyl, preferably (C₂-C₄)-alkenyl or (C₂-C₄)-haloalkenyl.

In a further embodiment, X is (C₁-C₄)-alkyl, in particular n-propyl, isopropyl, ethyl or methyl which may be substituted by one or more cyano and/or alkoxy groups.

In a further embodiment, X is cyano-(C₁-C₄)-alkyl, preferably cyano-(C₁-C₂)-alkyl, in particular —CH₂—CN.

In one embodiment of the invention, X is (C₁-C₄)-alkoxy-(C₁-C₄)-alkyl, in particular (C₁-C₂)-alkoxy-(C₁-C₂)-alkyl, such as methoxymethyl, or (C₁-C₄)-alkyl, in particular n-propyl, ethyl or methyl, in particular if R¹ and R² are both hydrogen.

In the compounds of the formula (I) or the precursors thereof, Y is in particular hydrogen, halogen, preferably fluorine, chlorine or bromine, (C₁-C₄)-alkyl, (C₁-C₄)-haloalkyl, (C₃-C₆)-cycloalkyl or (C₃-C₆)-halocycloalkyl.

According to a preferred embodiment of the present invention, Y is hydrogen.

According to a further preferred embodiment of the present invention, Y is halogen, preferably fluorine, chlorine or bromine.

According to a further preferred embodiment of the present invention, Y is (C₁-C₄)-alkyl or (C₁-C₄)-haloalkyl, preferably (C₁-C₂)-alkyl or (C₁-C₂)-haloalkyl, in particular methyl or ethyl, which may be substituted by one, two or three halogen atoms.

According to a further preferred embodiment of the present invention, Y is (C₃-C₆)-cycloalkyl or (C₃-C₆)-halocycloalkyl, particularly preferably cyclopropyl or halocyclopropyl which may carry one to three halogen atoms.

According to a further preferred embodiment, Y is NH₂. From among these compounds, particular preference is given to compounds in which R¹═R²=hydrogen.

Furthermore, X in these compounds is preferably (C₁-C₄)-alkyl, (C₁-C₂)-alkoxy-(C₁-C₄)-alkyl, in particular methyl, ethyl, n-propyl or methoxymethyl.

Besides, R⁵ and R⁶ independently of one another are preferably hydrogen or (C₁-C₄)-alkyl.

Furthermore, A¹ is preferably hydrogen, (C₁-C₆)-alkyl or amino. The index n is preferably 0, 1 or 2.

A² is preferably (C₁-C₄)-alkoxy, NH₂, (C₁-C₄)-alkylamine or di-(C₁-C₄)-alkylamino.

Examples of preferred compounds of the formula (I) are the compounds (Ia), (Ib), (Ic), (Id), (Ie), (If, (Ig) and (Ih) compiled in Tables 1 to 156 below. The groups mentioned in Tables 1 to 156 for a substituent are furthermore per se, independently of the combination in which they are mentioned, a particularly preferred embodiment of the substituent in question.

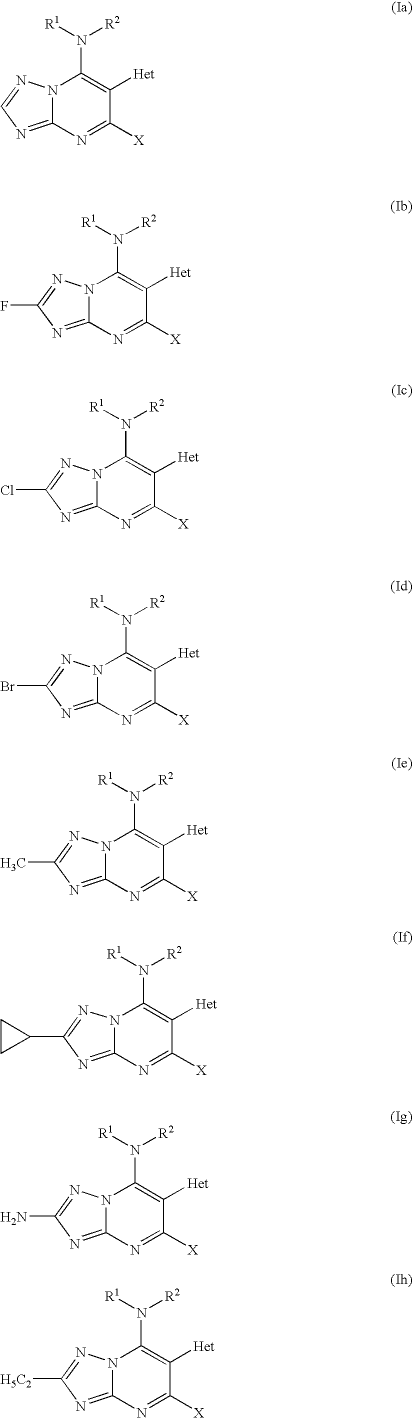

Table 1
Compounds of the formulae (Ia), (Ib), (Ic), (Id), (Ie), (If), (Ig)
and (Ih) in which Het is 3-methyl-5-isopropylpyrazol-1-yl and the
combination of R¹, R² and X for a compound corresponds in each
case to one row of Table B
Table 2
Compounds of the formulae (Ia), (Ib), (Ic), (Id), (Ie), (If), (Ig)
and (Ih) in which Het is 3,5-dimethylpyrazol-1-yl and the
combination of R¹, R² and X for a compound corresponds in each
case to one row of Table B
Table 3
Compounds of the formulae (Ia), (Ib), (Ic), (Id), (Ie), (If), (Ig)
and (Ih) in which Het is 3-isopropyl-5-methylpyrazol-1-yl and the
combination of R¹, R² and X for a compound corresponds in each
case to one row of Table B
Table 4
Compounds of the formulae (Ia), (Ib), (Ic), (Id), (Ie), (If), (Ig)
and (Ih) in which Het is 3-ethyl-5-methylpyrazol-1-yl and the
combination of R¹, R² and X for a compound corresponds in each
case to one row of Table B
Table 5
Compounds of the formulae (Ia), (Ib), (Ic), (Id), (Ie), (If), (Ig)
and (Ih) in which Het is 3-methyl-5-methoxypyrazol-1-yl and the
combination of R¹, R² and X for a compound corresponds in each
case to one row of Table B
Table 6
Compounds of the formulae (Ia), (Ib), (Ic), (Id), (Ie), (If), (Ig)
and (Ih) in which Het is 3,4,5-trimethylpyrazol-1-yl and the
combination of R¹, R² and X for a compound corresponds in each
case to one row of Table B
Table 7
Compounds of the formulae (Ia), (Ib), (Ic), (Id), (Ie), (If), (Ig)
and (Ih) in which Het is 3,5-dimethyl-4-chloropyrazol-1-yl and
the combination of R¹, R² and X for a compound corresponds in each
case to one row of Table B
Table 8
Compounds of the formulae (Ia), (Ib), (Ic), (Id) (Ie), (If), (Ig)
and (Ih) in which Het is 3-chloropyrazol-1-yl and the combination
of R¹, R² and X for a compound corresponds in each case to one
row of Table B
Table 9
Compounds of the formulae (Ia), (Ib), (Ic), (Id), (Ie), (If), (Ig)
and (Ih) in which Het is 3,4-dichloro-5-trichloromethylpyrazol-1-yl
and the combination of R¹, R² and X for a compound corresponds in
each case to one row of Table B
Table 10
Compounds of the formulae (Ia), (Ib), (Ic), (Id), (Ie), (If), (Ig)
and (Ih) in which Het is 3-methylpyrazol-1-yl and the combination
of R¹, R² and X for a compound corresponds in each case to one
row of Table B
Table 11
Compounds of the formulae (Ia), (Ib), (Ic), (Id), (Ie), (If), (Ig)
and (Ih) in which Het is 3,5-dichloro-4-methylpyrazol-1-yl and
the combination of R¹, R² and X for a compound corresponds in
each case to one row of Table B
Table 12
Compounds of the formulae (Ia), (Ib), (Ic), (Id), (Ie), (If), (Ig)
and (Ih) in which Het is 3-methyl-4-chloropyrazol-1-yl and the
combination of R¹, R² and X for a compound corresponds in each
case to one row of Table B
Table 13
Compounds of the formulae (Ia), (Ib), (Ic), (Id), (Ie), (If), (Ig)
and (Ih) in which Het is 1,3-dimethylpyrazol-5-yl and the
combination of R¹, R² and X for a compound corresponds in each
case to one row of Table B
Table 14
Compounds of the formulae (Ia), (Ib), (Ic), (Id), (Ie), (If), (Ig)
and (Ih) in which Het is 1-methyl-3-trifluoromethylpyrazol-5-yl
and the combination of R¹, R² and X for a compound corresponds in
each case to one row of Table B
Table 15
Compounds of the formulae (Ia), (Ib), (Ic), (Id), (Ie), (If), (Ig)
and (Ih) in which Het is 1,5-dimethylpyrazol-3-yl and the
combination of R¹, R² for X for a compound corresponds in each
case to one row of Table B
Table 16
Compounds of the formulae (Ia), (Ib), (Ic), (Id), (Ie), (If), (Ig)
and (Ih) in which Het is 1-methyl-5-methoxypyrazol-3-yl and the
combination of R¹, R² and X for a compound corresponds in each
case to one row of Table B
Table 17
Compounds of the formulae (Ia), (Ib), (Ic), (Id), (Ie), (If), (Ig)
and (Ih) in which Het is 1,3,5-trimethylpyrazol-4-yl and the
combination of R¹, R² and X for a compound corresponds in each
case to one row of Table B
Table 18
Compounds of the formulae (Ia), (Ib), (Ic), (Id), (Ie), (If), (Ig)
and (Ih) in which Het is 1-methyl-3-trifluoromethylpyrazol-4-yl
and the combination of R¹, R² and X for a compound corresponds
in each case to one row of Table B
Table 19
Compounds of the formulae (Ia), (Ib), (Ic), (Id), (Ie), (If), (Ig)
and (Ih) in which Het is 1,3-dimethylpyrazol-4-yl and the
combination of R¹, R² and X for a compound corresponds in each
case to one row of Table B
Table 20
Compounds of the formulae (Ia), (Ib), (Ic), (Id), (Ie), (If), (Ig)
and (Ih) in which Het is 1-methyl-5-trifluoromethylpyrazol-4-yl
and the combination of R¹, R² and X for a compound corresponds
in each case to one row of Table B
Table 21
Compounds of the formulae (Ia), (Ib), (Ic), (Id), (Ie), (If), (Ig)
and (Ih) in which Het is 1,5-dimethylpyrazol-4-yl and the
combination of R¹, R² and X for a compound corresponds in each
case to one row of Table B
Table 22
Compounds of the formulae (Ia), (Ib), (Ic), (Id), (Ie), (If), (Ig)
and (Ih) in which Het is 1-methylpyrrol-2-yl and the combination
of R¹, R² and X for a compound corresponds in each case to one
row of Table B
Table 23
Compounds of the formulae (Ia), (Ib), (Ic), (Id), (Ie), (If), (Ig)
and (Ih) in which Het is 1,4-dimethylpyrrol-2-yl and the
combination of R¹, R² and X for a compound corresponds in each
case to one row of Table B
Table 24
Compounds of the formulae (Ia), (Ib), (Ic), (Id), (Ie), (If), (Ig)
and (Ih) in which Het is 1-methyl-5-chloropyrrol-2-yl and the
combination of R¹, R² and X for a compound corresponds in each
case to one row of Table B
Table 25
Compounds of the formulae (Ia), (Ib), (Ic), (Id), (Ie), (If), (Ig)
and (Ih) in which Het is 1-methyl-3,5-dichloropyrrol-2-yl and
the combination of R¹, R² and X for a compound corresponds in
each case to one row of Table B
Table 26
Compounds of the formulae (Ia), (Ib), (Ic), (Id), (Ie), (If), (Ig)
and (Ih) in which Het is 2-methylthiazol-4-yl and the combination
of R¹, R² and X for a compound corresponds in each case to one
row of Table B
Table 27
Compounds of the formulae (Ia), (Ib), (Ic), (Id), (Ie), (If), (Ig)
and (Ih) in which Het is thiazol-4-yl and the combination of
R¹, R² and X for a compound corresponds in each case to one
row of Table B
Table 28
Compounds of the formulae (Ia), (Ib), (Ic), (Id), (Ie), (If), (Ig)
and (Ih) in which Het is 2-methyl-5-chlorothiazol-4-yl and the
combination of R¹, R² and X for a compound corresponds in each
case to one row of Table B
Table 29
Compounds of the formulae (Ia), (Ib), (Ic), (Id), (Ie), (If), (Ig)
and (Ih) in which Het is 2,5-dichlorothiazol-4-yl and the
combination of R¹, R² and X for a compound corresponds in each
case to one row of Table B
Table 30
Compounds of the formulae (Ia), (Ib), (Ic), (Id), (Ie), (If) (Ig)
and (Ih) in which Het is 2-methyl-5-bromothiazol-4-yl and the
combination of R¹, R² and X for a compound corresponds in each
case to one row of Table B
Table 31
Compounds of the formulae (Ia), (Ib), (Ic), (Id), (Ie), (If), (Ig)
and (Ih) in which Het is 3-methylisothiazol-4-yl and the
combination of R¹, R² and X for a compound corresponds in each
case to one row of Table B
Table 32
Compounds of the formulae (Ia), (Ib), (Ic), (Id), (Ie), (If), (Ig)
and (Ih) in which Het is 3-methyl-5-chloroisothiazol-4-yl and the
combination of R¹, R² and X for a compound corresponds in each
case to one row of Table B
Table 33
Compounds of the formulae (Ia), (Ib), (Ic), (Id), (Ie), (If), (Ig)
and (Ih) in which Het is isoxazol-4-yl and the combination of
R¹, R² and X for a compound corresponds in each case to one row
of Table B
Table 34
Compounds of the formulae (Ia), (Ib), (Ic), (Id), (Ie), (If) (Ig)
and (Ih) in which Het is 3,5-dimethylisoxazol-4-yl and the
combination of R¹, R² and X for a compound corresponds in each
case to one row of Table B
Table 35
Compounds of the formulae (Ia), (Ib), (Ic), (Id), (Ie), (If), (Ig)
and (Ih) in which Het is 3-chloroisoxazol-4-yl and the
combination of R¹, R² and X for a compound corresponds in each
case to one row of Table B
Table 36
Compounds of the formulae (Ia), (Ib), (Ic), (Id), (Ie) (If), (Ig)
and (Ih) in which Het is 3-methylisoxazol-4-yl and the
combination of R¹, R² and X for a compound corresponds in each
case to one row of Table B
Table 37
Compounds of the formulae (Ia), (Ib) (Ic), (Id), (Ie), (If), (Ig)
and (Ih) in which Het is oxazol-4-yl and the combination of
R¹, R² and X for a compound corresponds in each case to one row
of Table B
Table 38
Compounds of the formulae (Ia), (Ib), (Ic), (Id), (Ie), (If), (Ig)
and (Ih) in which Het is 2,5-dimethyloxazol-4-yl and the
combination of R¹, R² and X for a compound corresponds in each
case to one row of Table B
Table 39
Compounds of the formulae (Ia), (Ib), (Ic), (Id) (Ie), (If), (Ig)
and (Ih) in which Het is 2-methyloxazol-4-yl and the combination
of R¹, R² and X for a compound corresponds in each case to one
row of Table B
Table 40
Compounds of the formulae (Ia), (Ib), (Ic), (Id), (Ie), (If), (Ig)
and (Ih) in which Het is 4,5-dichloroimidazol-1-yl and the
combination of R¹, R² and X for a compound corresponds in each
case to one row of Table B
Table 41
Compounds of the formulae (Ia), (Ib), (Ic), (Id), (Ie), (If), (Ig)
and (Ih) in which Het is 4,5-dimethylimidazol-1-yl and the
combination of R¹, R² and X for a compound corresponds in each
case to one row of Table B
Table 42
Compounds of the formulae (Ia), (Ib), (Ic), (Id), (Ie), (If), (Ig)
and (Ih) in which Het is 3,5-dimethyl-1,2,4-triazol-1-yl and the
combination of R¹, R² and X for a compound corresponds in each
case to one row of Table B
Table 43
Compounds of the formulae (Ia), (Ib), (Ic), (Id), (Ie), (If), (Ig)
and (Ih) in which Het is 3,5-dichloro-1,2,4-triazol-1-yl and the
combination of R¹, R² and X for a compound corresponds in each
case to one row of Table B
Table 44
Compounds of the formulae (Ia), (Ib), (Ic), (Id), (Ie), (If), (Ig)
and (Ih) in which Het is 3,5-dibromo-1,2,4-triazol-1-yl and the
combination of R¹, R² and X for a compound corresponds in each
case to one row of Table B
Table 45
Compounds of the formulae (Ia), (Ib), (Ic), (Id), (Ie), (If), (Ig)
and (Ih) in which Het is 3,5-difluoro-1,2,4-triazol-1-yl and the
combination of R¹, R² and X for a compound corresponds in each
case to one row of Table B
Table 46
Compounds of the formulae (Ia), (Ib), (Ic), (Id), (Ie), (If), (Ig)
and (Ih) in which Het is 3,5-ditrifluoromethyl-1,2,4-triazol-1-yl
and the combination of R¹, R² and X for a compound corresponds
in each case to one row of Table B
Table 47
Compounds of the formulae (Ia), (Ib), (Ic), (Id), (Ie), (If), (Ig)
and (Ih) in which Het is 3-methyl-1,2,4-triazol-1-yl and the
combination of R¹, R² and X for a compound corresponds in each
case to one row of Table B
Table 48
Compounds of the formulae (Ia), (Ib), (Ic), (Id), (Ie), (If), (Ig)
and (Ih) in which Het is 3-chloro-1,2,4-triazol-1-yl and the
combination of R¹, R² and X for a compound corresponds in each
case to one row of Table B
Table 49
Compounds of the formulae (Ia), (Ib), (Ic), (Id), (Ie), (If), (Ig)
and (Ih) in which Het is 3-fluoro-1,2,4-triazol-1-yl and the
combination of R¹, R² and X for a compound corresponds in each
case to one row of Table B
Table 50
Compounds of the formulae (Ia), (Ib), (Ic), (Id), (Ie), (If), (Ig)
and (Ih) in which Het is 3-bromo-1,2,4-triazol-1-yl and the
combination of R¹, R² and X for a compound corresponds in each
case to one row of Table B
Table 51
Compounds of the formulae (Ia), (Ib), (Ic), (Id), (Ie), (If), (Ig)
and (Ih) in which Het is 3-trifluoromethyl-1,2,4-triazol-1-yl and
the combination of R¹, R² and X for a compound corresponds in
each case to one row of Table B
Table 52
Compounds of the formulae (Ia), (Ib), (Ic), (Id), (Ie), (If), (Ig)
and (Ih) in which Het is 4,5-dimethyl-1,2,3-triazol-1-yl and the
combination of R¹, R² and X for a compound corresponds in each
case to one row of Table B
Table 53
Compounds of the formulae (Ia), (Ib), (Ic), (Id), (Ie), (If), (Ig)
and (Ih) in which Het is 4,5-dichloro-1,2,3-triazol-1-yl and the
combination of R¹, R² and X for a compound corresponds in each
case to one row of Table B
Table 54
Compounds of the formulae (Ia), (Ib), (Ic), (Id), (Ie), (If), (Ig)
and (Ih) in which Het is 4,5-dibromo-1,2,3-triazol-1-yl and the
combination of R¹, R² and X for a compound corresponds in each
case to one row of Table B
Table 55
Compounds of the formulae (Ia), (Ib), (Ic), (Id), (Ie), (If), (Ig)
and (Ih) in which Het is 4,5-difluoro-1,2,3-triazol-1-yl and the
combination of R¹, R² and X for a compound corresponds in each
case to one row of Table B
Table 56
Compounds of the formulae (Ia), (Ib), (Ic), (Id), (Ie), (If), (Ig)
and (Ih) in which Het is 4,5-ditrifluoromethyl-1,2,3-triazol-1-yl
and the combination of R¹, R² and X for a compound corresponds in
each case to one row of Table B
Table 57
Compounds of the formulae (Ia), (Ib), (Ic), (Id), (Ie), (If), (Ig)
and (Ih) in which Het is 5-methyl-1,2,3-triazol-1-yl and the
combination of R¹, R² and X for a compound corresponds in each
case to one row of Table B
Table 58
Compounds of the formulae (Ia), (Ib), (Ic), (Id), (Ie), (If), (Ig)
and (Ih) in which Het is 5-chloro-1,2,3-triazol-1-yl and the
combination of R¹, R² and X for a compound corresponds in each
case to one row of Table B
Table 59
Compounds of the formulae (Ia), (Ib), (Ic), (Id), (Ie), (If), (Ig)
and (Ih) in which Het is 5-fluoro-1,2,3-triazol-1-yl and the
combination of R¹, R² and X for a compound corresponds in each
case to one row of Table B
Table 60
Compounds of the formulae (Ia), (Ib), (Ic), (Id), (Ie), (If), (Ig)
and (Ih) in which Het is 5-bromo-1,2,3-triazol-1-yl and the
combination of R¹, R² and X for a compound corresponds in each
case to one row of Table B
Table 61
Compounds of the formulae (Ia), (Ib), (Ic), (Id), (Ie), (If), (Ig)
and (Ih) in which Het is 5-trifluoromethyl-1,2,3-triazol-1-yl and
the combination of R¹, R² and X for a compound corresponds in
each case to one row of Table B
Table 62
Compounds of the formulae (Ia), (Ib), (Ic), (Id), (Ie), (If), (Ig)
and (Ih) in which Het is 2-thienyl and the combination of R¹, R²
and X for a compound corresponds in each case to one row of
Table B
Table 63
Compounds of the formulae (Ia), (Ib), (Ic), (Id), (Ie), (If), (Ig)
and (Ih) in which Het is 3,5-dichlorothiophen-2-yl and the
combination of R¹, R² and X for a compound corresponds in each
case to one row of Table B
Table 64
Compounds of the formulae (Ia), (Ib), (Ic), (Id), (Ie), (If), (Ig)
and (Ih) in which Het is 3,4,5-trichlorothiophen-2-yl and the
combination of R¹, R² and X for a compound corresponds in each
case to one row of Table B
Table 65
Compounds of the formulae (Ia), (Ib), (Ic), (Id), (Ie), (If), (Ig)
and (Ih) in which Het is 5-chlorothiophen-2-yl and the
combination of R¹, R² and X for a compound corresponds in each
case to one row of Table B
Table 66
Compounds of the formulae (Ia), (Ib), (Ic), (Id), (Ie), (If), (Ig)
and (Ih) in which Het is 5-bromothiophen-2-yl and the
combination of R¹, R² and X for a compound corresponds in each
case to one row of Table B
Table 67
Compounds of the formulae (Ia), (Ib), (Ic), (Id), (Ie), (If), (Ig)
and (Ih) in which Het is 5-methylthiophen-2-yl and the
combination of R¹, R² and X for a compound corresponds in each
case to one row of Table B
Table 68
Compounds of the formulae (Ia), (Ib), (Ic), (Id), (Ie), (If), (Ig)
and (Ih) in which Het is 2,5-dichlorothiophen-3-yl and the
combination of R¹, R² and X for a compound corresponds in each
case to one row of Table B
Table 69
Compounds of the formulae (Ia), (Ib), (Ic), (Id), (Ie), (If), (Ig)
and (Ih) in which Het is 2,5-dibromothiophen-3-yl and the
combination of R¹, R² and X for a compound corresponds in each
case to one row of Table B
Table 70
Compounds of the formulae (Ia), (Ib), (Ic), (Id), (Ie), (If), (Ig)
and (Ih) in which Het is 2-methylthiophen-3-yl and the
combination of R¹, R² and X for a compound corresponds in each
case to one row of Table B
Table 71
Compounds of the formulae (Ia), (Ib), (Ic), (Id), (Ie), (If), (Ig)
and (Ih) in which Het is 5-methyltetrazol-1-yl and the
combination of R¹, R² and X for a compound corresponds in each
case to one row of Table B
Table 72
Compounds of the formulae (Ia), (Ib), (Ic), (Id), (Ie), (If), (Ig)
and (Ih) in which Het is 5-methyltetrazol-2-yl and the
combination of R¹, R² and X for a compound corresponds in each
case to one row of Table B
Table 73
Compounds of the formulae (Ia), (Ib), (Ic), (Id), (Ie), (If), (Ig)
and (Ih) in which Het is 5-chlorotetrazol-1-yl and the
combination of R¹, R² and X for a compound corresponds in each
case to one row of Table B
Table 74
Compounds of the formulae (Ia), (Ib), (Ic), (Id), (Ie), (If), (Ig)
and (Ih) in which Het is 5-chlorotetrazol-2-yl and the
combination of R¹, R² and X for a compound corresponds in each
case to one row of Table B
Table 75
Compounds of the formulae (Ia), (Ib), (Ic), (Id), (Ie), (If), (Ig)
and (Ih) in which Het is 5-bromotetrazol-1-yl and the
combination of R¹, R² and X for a compound corresponds in each
case to one row of Table B
Table 76
Compounds of the formulae (Ia), (Ib), (Ic), (Id), (Ie), (If), (Ig)
and (Ih) in which Het is 5-bromotetrazol-2-yl and the
combination of R¹, R² and X for a compound corresponds in each
case to one row of Table B
Table 77
Compounds of the formulae (Ia), (Ib), (Ic), (Id), (Ie), (If), (Ig)
and (Ih) in which Het is 1-methyltetrazol-5-yl and the
combination of R¹, R² and X for a compound corresponds in each
case to one row of Table B
Table 78
Compounds of the formulae (Ia), (Ib), (Ic), (Id), (Ie), (If), (Ig)
and (Ih) in which Het is 2-methyltetrazol-5-yl and the
combination of R¹, R² and X for a compound corresponds in each
case to one row of Table B
Table 79
Compounds of the formulae (Ia), (Ib), (Ic), (Id), (Ie), (If), (Ig)
and (Ih) in which Het is 5-methyl-[1,3,4]-thiadiazol-2-yl
and the combination of R¹, R² and X for a compound corresponds
in each case to one row of Table B
Table 80
Compounds of the formulae (Ia), (Ib), (Ic), (Id), (Ie), (If), (Ig)
and (Ih) in which Het is 5-chloro-[1,3,4]-thiadiazol-2-yl
and the combination of R¹, R² and X for a compound corresponds
in each case to one row of Table B
Table 81
Compounds of the formulae (Ia), (Ib), (Ic), (Id) (Ie), (If), (Ig)
and (Ih) in which Het is 1,3,4-thiadiazol-2-yl and the
combination of R¹, R² and X for a compound corresponds in each
case to one row of Table B
Table 82
Compounds of the formulae (Ia), (Ib), (Ic), (Id), (Ie), (If), (Ig)
and (Ih) in which Het is 5-bromo-[1,3,4]-thiadiazol-2-yl
and the combination of R¹, R² and X for a compound corresponds
in each case to one row of Table B
Table 83
Compounds of the formulae (Ia), (Ib), (Ic), (Id) (Ie), (If), (Ig)
and (Ih) in which Het is 1,2,3-thiadiazol-4-yl and the
combination of R¹, R² and X for a compound corresponds in each
case to one row of Table B
Table 84
Compounds of the formulae (Ia), (Ib), (Ic), (Id), (Ie), (If), (Ig)
and (Ih) in which Het is 5-methyl-[1,3,4]-thiadiazol-4-yl
and the combination of R¹, R² and X for a compound corresponds
in each case to one row of Table B
Table 85
Compounds of the formulae (Ia), (Ib), (Ic), (Id), (Ie), (If), (Ig)
and (Ih) in which Het is 5-chloro-[1,2,3]-thiadiazol-4-yl
and the combination of R¹, R² and X for a compound corresponds
in each case to one row of Table B
Table 86
Compounds of the formulae (Ia), (Ib), (Ic), (Id), (Ie), (If), (Ig)
and (Ih) in which Het is 5-bromo-[1,2,3]-thiadiazol-4-yl
and the combination of R¹, R² and X for a compound corresponds
in each case to one row of Table B
Table 87
Compounds of the formulae (Ia), (Ib), (Ic), (Id), (Ie), (If), (Ig)
and (Ih) in which Het is 1,2,3-thiadiazol-5-yl and the
combination of R¹, R² and X for a compound corresponds in each
case to one row of Table B
Table 88
Compounds of the formulae (Ia), (Ib), (Ic), (Id), (Ie), (If), (Ig)
and (Ih) in which Het is 4-methyl-[1,2,3]-thiadiazol-5-yl
and the combination of R¹, R² and X for a compound corresponds
in each case to one row of Table B
Table 89
Compounds of the formulae (Ia), (Ib), (Ic), (Id), (Ie), (If), (Ig)
and (Ih) in which Het is 4-chloro-[1,2,3]-thiadiazol-5-yl
and the combination of R¹, R² and X for a compound corresponds
in each case to one row of Table B
Table 90
Compounds of the formulae (Ia), (Ib), (Ic), (Id), (Ie), (If), (Ig)
and (Ih) in which Het is bromo-[1,2,3]-thiadiazol-5-yl and
the combination of R¹, R² and X for a compound corresponds in
each case to one row of Table B
Table 91
Compounds of the formulae (Ia), (Ib), (Ic), (Id), (Ie), (If), (Ig)
and (Ih) in which Het is 1,2,4-thiadiazol-5-yl and the
combination of R¹, R² and X for a compound corresponds in each
case to one row of Table B
Table 92
Compounds of the formulae (Ia), (Ib), (Ic), (Id), (Ie), (If), (Ig)
and (Ih) in which Het is 3-methyl-[1,2,4]-thiadiazol-5-yl
and the combination of R¹, R² and X for a compound corresponds
in each case to one row of Table B
Table 93
Compounds of the formulae (Ia), (Ib), (Ic), (Id), (Ie), (If), (Ig)
and (Ih) in which Het is 3-chloro-[1,2,4]-thiadiazol-5-yl
and the combination of R¹, R² and X for a compound corresponds
in each case to one row of Table B
Table 94
Compounds of the formulae (Ia), (Ib), (Ic), (Id), (Ie), (If), (Ig)
and (Ih) in which Het is 3-bromo-[1,2,4]-thiadiazol-5-yl
and the combination of R¹, R² and X for a compound corresponds
in each case to one row of Table B
Table 95
Compounds of the formulae (Ia), (Ib), (Ic), (Id), (Ie), (If), (Ig)
and (Ih) in which Het is 1,2,4-thiadiazol-3-yl and the
combination of R¹, R² and X for a compound corresponds in each
case to one row of Table B
Table 96
Compounds of the formulae (Ia), (Ib), (Ic), (Id), (Ie), (If), (Ig)
and (Ih) in which Het is 5-methyl-[1,2,4]-thiadiazol-3-yl
and the combination of R¹, R² and X for a compound corresponds
in each case to one row of Table B
Table 97
Compounds of the formulae (Ia), (Ib), (Ic), (Id), (Ie), (If), (Ig)
and (Ih) in which Het is 5-chloro-[1,2,4]-thiadiazol-3-yl
and the combination of R¹, R² and X for a compound corresponds
in each case to one row of Table B
Table 98
Compounds of the formulae (Ia), (Ib), (Ic), (Id), (Ie), (If), (Ig)
and (Ih) in which Het is 5-bromo-[1,2,4]-thiadiazol-3-yl
and the combination of R¹, R² and X for a compound corresponds
in each case to one row of Table B
Table 99
Compounds of the formulae (Ia), (Ib), (Ic), (Id), (Ie), (If), (Ig)
and (Ih) in which Het is 1,2,5-thiadiazol-3-yl and the
combination of R¹, R² and X for a compound corresponds in each
case to one row of Table B
Table 100
Compounds of the formulae (Ia), (Ib), (Ic), (Id), (Ie), (If), (Ig)
and (Ih) in which Het is 4-methyl-[1,2,5]-thiadiazol-3-yl
and the combination of R¹, R² and X for a compound corresponds
in each case to one row of Table B
Table 101
Compounds of the formulae (Ia), (Ib), (Ic), (Id), (Ie), (If), (Ig)
and (Ih) in which Het is 4-chloro-[1,2,5]-thiadiazol-3-yl
and the combination of R¹, R² and X for a compound corresponds
in each case to one row of Table B
Table 102
Compounds of the formulae (Ia), (Ib), (Ic), (Id), (Ie), (If), (Ig)
and (Ih) in which Het is 4-bromo-[1,2,5]-thiadiazol-3-yl
and the combination of R¹, R² and X for a compound corresponds
in each case to one row of Table B
Table 103
Compounds of the formulae (Ia), (Ib), (Ic) (Id), (Ie), (If), (Ig)
and (Ih) in which Het is pyrazol-1-yl and the combination of
R¹, R² and X for a compound corresponds in each case to one row
of Table B
Table 104
Compounds of the formulae (Ia), (Ib), (Ic), (Id), (Ie), (If), (Ig)
and (Ih) in which Het is 3-ethylpyrazol-1-yl and the combination
of R¹, R² and X for a compound corresponds in each case to one
row of Table B
Table 105
Compounds of the formulae (Ia), (Ib), (Ic), (Id), (Ie), (If), (Ig)
and (Ih) in which Het is 4-ethylpyrazol-1-yl and the combination
of R¹, R² and X for a compound corresponds in each case to one
row of Table B
Table 106
Compounds of the formulae (Ia), (Ib), (Ic), (Id), (Ie), (If), (Ig)
and (Ih) in which Het is 5-ethylpyrazol-1-yl and the combination
of R¹, R² and X for a compound corresponds in each case to one
row of Table B
Table 107
Compounds of the formulae (Ia), (Ib), (Ic), (Id), (Ie), (If), (Ig)
and (Ih) in which Het is 3-propylpyrazol-1-yl and the combination
of R¹, R² and X for a compound corresponds in each case to one
row of Table B
Table 108
Compounds of the formulae (Ia), (Ib), (Ic), (Id), (Ie), (If), (Ig)
and (Ih) in which Het is 4-propylpyrazol-1-yl and the combination
of R¹, R² and X for a compound corresponds in each case to one
row of Table B
Table 109
Compounds of the formulae (Ia), (Ib), (Ic), (Id), (Ie), (If), (Ig)
and (Ih) in which Het is 5-propylpyrazol-1-yl and the combination
of R¹, R² and X for a compound corresponds in each case to one
row of Table B
Table 110
Compounds of the formulae (Ia), (Ib), (Ic), (Id), (Ie), (If), (Ig)
and (Ih) in which Het is 3-butylpyrazol-1-yl and the combination
of R¹, R² and X for a compound corresponds in each case to one
row of Table B
Table 111
Compounds of the formulae (Ia), (Ib), (Ic), (Id), (Ie), (If), (Ig)
and (Ih) in which Het is 4-butylpyrazol-1-yl and the combination
of R¹, R² and X for a compound corresponds in each case to one
row of Table B
Table 112
Compounds of the formulae (Ia), (Ib), (Ic), (Id), (Ie), (If), (Ig)
and (Ih) in which Het is 5-butylpyrazol-1-yl and the combination
of R¹, R² and X for a compound corresponds in each case to one
row of Table B
Table 113
Compounds of the formulae (Ia), (Ib), (Ic), (Id) (Ie), (If), (Ig)
and (Ih) in which Het is 1,2,4-triazol-1-yl and the combination
of R¹, R² and X for a compound corresponds in each case to one
row of Table B
Table 114
Compounds of the formulae (Ia), (Ib), (Ic), (Id), (Ie), (If), (Ig)
and (Ih) in which Het is 3-ethyl-1,2,4-triazol-1-yl and the
combination of R¹, R² and X for a compound corresponds in each
case to one row of Table B
Table 115
Compounds of the formulae (Ia), (Ib), (Ic), (Id), (Ie), (If), (Ig)
and (Ih) in which Het is 5-ethyl-1,2,4-triazol-1-yl and the
combination of R¹, R² and X for a compound corresponds in each
case to one row of Table B
Table 116
Compounds of the formulae (Ia), (Ib), (Ic), (Id), (Ie), (If), (Ig)
and (Ih) in which Het is 3-propyl-1,2,4-triazol-1-yl and the
combination of R¹, R² and X for a compound corresponds in each
case to one row of Table B
Table 117
Compounds of the formulae (Ia), (Ib), (Ic), (Id), (Ie), (If), (Ig)
and (Ih) in which Het is 5-propyl-1,2,4-triazol-1-yl and the
combination of R¹, R² and X for a compound corresponds in each
case to one row of Table B
Table 118
Compounds of the formulae (Ia), (Ib), (Ic), (Id), (Ie), (If), (Ig)
and (Ih) in which Het is 3-butyl-1,2,4-triazol-1-yl and the
combination of R¹, R² and X for a compound corresponds in each
case to one row of Table B
Table 119
Compounds of the formulae (Ia), (Ib), (Ic), (Id), (Ie), (If), (Ig)
and (Ih) in which Het is 5-butyl-1,2,4-triazol-1-yl and the
combination of R¹, R² and X for a compound corresponds in each
case to one row of Table B
Table 120
Compounds of the formulae (Ia), (Ib), (Ic), (Id), (Ie), (If), (Ig)
and (Ih) in which Het is 1,2,3-triazol-1-yl and the combination
of R¹, R² and X for a compound corresponds in each case to one
row of Table B
Table 121
Compounds of the formulae (Ia), (Ib), (Ic), (Id), (Ie), (If), (Ig)
and (Ih) in which Het is 4-ethyl-1,2,3-triazol-1-yl and the
combination of R¹, R² and X for a compound corresponds in each
case to one row of Table B
Table 122
Compounds of the formulae (Ia), (Ib), (Ic), (Id), (Ie), (If), (Ig)
and (Ih) in which Het is 5-ethyl-1,2,3-triazol-1-yl and the
combination of R¹, R² and X for a compound corresponds in each
case to one row of Table B
Table 123
Compounds of the formulae (Ia), (Ib), (Ic), (Id), (Ie), (If), (Ig)
and (Ih) in which Het is 4-propyl-1,2,3-triazol-1-yl and the
combination of R¹, R² and X for a compound corresponds in each
case to one row of Table B
Table 124
Compounds of the formulae (Ia), (Ib), (Ic), (Id), (Ie), (If), (Ig)
and (Ih) in which Het is 5-propyl-1,2,3-triazol-1-yl and the
combination of R¹, R² and X for a compound corresponds in each
case to one row of Table B
Table 125
Compounds of the formulae (Ia), (Ib), (Ic), (Id), (Ie), (If), (Ig)
and (Ih) in which Het is 4-butyl-1,2,3-triazol-1-yl and the
combination of R¹, R² and X for a compound corresponds in each
case to one row of Table B
Table 126
Compounds of the formulae (Ia), (Ib), (Ic), (Id), (Ie), (If), (Ig)
and (Ih) in which Het is 5-butyl-1,2,3-triazol-1-yl and the
combination of R¹, R² and X for a compound corresponds in each
case to one row of Table B
Table 127
Compounds of the formulae (Ia), (Ib), (Ic), (Id), (Ie), (If), (Ig)
and (Ih) in which Het is thiazol-2-yl and the combination of R¹,
R² and X for a compound corresponds in each case to one row of
Table B
Table 128
Compounds of the formulae (Ia), (Ib), (Ic), (Id), (Ie), (If), (Ig)
and (Ih) in which Het is 4-ethylthiazol-2-yl and the combination
of R¹, R² and X for a compound corresponds in each case to one
row of Table B
Table 129
Compounds of the formulae (Ia), (Ib), (Ic), (Id), (Ie), (If), (Ig)
and (Ih) in which Het is 5-ethylthiazol-2-yl and the combination
of R¹, R² and X for a compound corresponds in each case to one
row of Table B
Table 130
Compounds of the formulae (Ia), (Ib), (Ic), (Id), (Ie), (If), (Ig)
and (Ih) in which Het is 4-propylthiazol-2-yl and the combination
of R¹, R² and X for a compound corresponds in each case to one
row of Table B
Table 131
Compounds of the formulae (Ia), (Ib), (Ic), (Id), (Ie), (If), (Ig)
and (Ih) in which Het is 5-propylthiazol-2-yl and the combination
of R¹, R² and X for a compound corresponds in each case to one
row of Table B
Table 132
Compounds of the formulae (Ia), (Ib), (Ic), (Id), (Ie), (If), (Ig)
and (Ih) in which Het is 4-butylthiazol-2-yl and the combination
of R¹, R² and X for a compound corresponds in each case to one
row of Table B
Table 133
Compounds of the formulae (Ia), (Ib), (Ic), (Id), (Ie), (If), (Ig)
and (Ih) in which Het is 5-butylthiazol-2-yl and the combination
of R¹, R² and X for a compound corresponds in each case to one
row of Table B
Table 134
Compounds of the formulae (Ia), (Ib), (Ic), (Id), (Ie), (If), (Ig)
and (Ih) in which Het is 2-ethylthiazol-4-yl and the combination
of R¹, R² and X for a compound corresponds in each case to one
row of Table B
Table 135
Compounds of the formulae (Ia), (Ib), (Ic), (Id), (Ie), (If), (Ig)
and (Ih) in which Het is 5-ethylthiazol-4-yl and the combination
of R¹, R² and X for a compound corresponds in each case to one
row of Table B
Table 136
Compounds of the formulae (Ia), (Ib), (Ic), (Id), (Ie), (If), (Ig)
and (Ih) in which Het is 2-propylthiazol-4-yl and the combination
of R¹, R² and X for a compound corresponds in each case to one
row of Table B
Table 137
Compounds of the formulae (Ia), (Ib), (Ic), (Id), (Ie), (If), (Ig)
and (Ih) in which Het is 5-propylthiazol-4-yl and the combination
of R¹, R² and X for a compound corresponds in each case to one
row of Table B
Table 138
Compounds of the formulae (Ia), (Ib), (Ic), (Id), (Ie), (If), (Ig)
and (Ih) in which Het is 2-butylthiazol-4-yl and the combination
of R¹, R² and X for a compound corresponds in each case to one
row of Table B
Table 139
Compounds of the formulae (Ia), (Ib), (Ic), (Id), (Ie), (If), (Ig)
and (Ih) in which Het is 5-butylthiazol-4-yl and the combination
of R¹, R² and X for a compound corresponds in each case to one
row of Table B
Table 140
Compounds of the formulae (Ia), (Ib), (Ic), (Id), (Ie), (If), (Ig)
and (Ih) in which Het is thiazol-5-yl and the combination of R¹,
R² and X for a compound corresponds in each case to one row of
Table B
Table 141
Compounds of the formulae (Ia), (Ib), (Ic), (Id), (Ie), (If), (Ig)
and (Ih) in which Het is 2-ethylthiazol-5-yl and the combination
of R¹, R² and X for a compound corresponds in each case to one
row of Table B
Table 142
Compounds of the formulae (Ia), (Ib), (Ic), (Id), (Ie), (If), (Ig)
and (Ih) in which Het is 4-ethylthiazol-5-yl and the combination
of R¹, R² and X for a compound corresponds in each case to one
row of Table B
Table 143
Compounds of the formulae (Ia), (Ib), (Ic), (Id), (Ie), (If), (Ig)
and (Ih) in which Het is 2-propylthiazol-5-yl and the combination
of R¹, R² and X for a compound corresponds in each case to one
row of Table B
Table 144
Compounds of the formulae (Ia), (Ib), (Ic), (Id), (Ie), (If), (Ig)
and (Ih) in which Het is 4-propylthiazol-5-yl and the combination
of R¹, R² and X for a compound corresponds in each case to one
row of Table B
Table 145
Compounds of the formulae (Ia), (Ib), (Ic), (Id), (Ie), (If), (Ig)
and (Ih) in which Het is 2-butylthiazol-5-yl and the combination
of R¹, R² and X for a compound corresponds in each case to one
row of Table B
Table 146
Compounds of the formulae (Ia), (Ib), (Ic), (Id), (Ie), (If), (Ig)
and (Ih) in which Het is 4-butylthiazol-5-yl and the combination
of R¹, R² and X for a compound corresponds in each case to one
row of Table B
Table 147
Compounds of the formulae (Ia), (Ib), (Ic), (Id), (Ie), (If), (Ig)
and (Ih) in which Het is 3-thienyl and the combination of R¹, R²
and X for a compound corresponds in each case to one row of
Table B
Table 148
Compounds of the formulae (Ia), (Ib), (Ic), (Id), (Ie), (If), (Ig)
and (Ih) in which Het is 2-ethylthien-3-yl and the combination of
R¹, R² and X for a compound corresponds in each case to one row
of Table B
Table 149
Compounds of the formulae (Ia), (Ib), (Ic), (Id), (Ie), (If), (Ig)
and (Ih) in which Het is 4-ethylthien-3-yl and the combination of
R¹, R² and X for a compound corresponds in each case to one row
of Table B
Table 150
Compounds of the formulae (Ia), (Ib), (Ic), (Id), (Ie), (If), (Ig)
and (Ih) in which Het is 5-ethylthien-3-yl and the combination
of R¹, R² and X for a compound corresponds in each case to one
row of Table B
Table 151
Compounds of the formulae (Ia), (Ib), (Ic), (Id), (Ie), (If), (Ig)
and (Ih) in which Het is 2-propylthien-3-yl and the combination
of R¹, R² and X for a compound corresponds in each case to one
row of Table B
Table 152
Compounds of the formulae (Ia), (Ib), (Ic), (Id), (Ie), (If), (Ig)
and (Ih) in which Het is 4-propylthien-3-yl and the combination
of R¹, R² and X for a compound corresponds in each case to one
row of Table B
Table 153
Compounds of the formulae (Ia), (Ib), (Ic), (Id), (Ie), (If), (Ig)
and (Ih) in which Het is 5-propylthien-3-yl and the combination
of R¹, R² and X for a compound corresponds in each case to one
row of Table B
Table 154
Compounds of the formulae (Ia), (Ib), (Ic), (Id), (Ie), (If), (Ig)
and (Ih) in which Het is 2-butylthien-3-yl and the combination
of R¹, R² and X for a compound corresponds in each case to one
row of Table B
Table 155
Compounds of the formulae (Ia), (Ib), (Ic), (Id), (Ie), (If), (Ig)
and (Ih) in which Het is 4-butylthien-3-yl and the combination
of R¹, R² and X for a compound corresponds in each case to one
row of Table B
Table 156
Compounds of the formulae (Ia), (Ib), (Ic), (Id), (Ie), (If), (Ig)
and (Ih) in which Het is 5-butylthien-3-yl and the combination
of R¹, R² and X for a compound corresponds in each case to one
row of Table B

TABLE B

| Row | R¹ | R² | X |
|---|---|---|---|
| B-1 | H | H | CH₃ |
| B-2 | CH₃ | H | CH₃ |
| B-3 | CH₃ | CH₃ | CH₃ |
| B-4 | CH₂CH₃ | H | CH₃ |
| B-5 | CH₂CH₃ | CH₃ | CH₃ |
| B-6 | CH₂CH₃ | CH₂CH₃ | CH₃ |
| B-7 | CH₂CF₃ | H | CH₃ |
| B-8 | CH₂CF₃ | CH₃ | CH₃ |
| B-9 | CH₂CF₃ | CH₂CH₃ | CH₃ |
| B-10 | CH₂CCl₃ | H | CH₃ |
| B-11 | CH₂CCl₃ | CH₃ | CH₃ |
| B-12 | CH₂CCl₃ | CH₂CH₃ | CH₃ |
| B-13 | CH₂CH₂CH₃ | H | CH₃ |
| B-14 | CH₂CH₂CH₃ | CH₃ | CH₃ |
| B-15 | CH₂CH₂CH₃ | CH₂CH₃ | CH₃ |
| B-16 | CH₂CH₂CH₃ | CH₂CH₂CH₃ | CH₃ |
| B-17 | CH(CH₃)₂ | H | CH₃ |
| B-18 | CH(CH₃)₂ | CH₃ | CH₃ |
| B-19 | CH(CH₃)₂ | CH₂CH₃ | CH₃ |
| B-20 | CH₂CH(CH₃)₂ | H | CH₃ |
| B-21 | CH₂CH(CH₃)₂ | CH₃ | CH₃ |
| B-22 | CH₂CH(CH₃)₂ | CH₂CH₃ | CH₃ |
| B-23 | CH₂CH(CH₃)₂ | CH₂CH₂CH₃ | CH₃ |
| B-24 | CH₂CH₂CH₂CH₃ | H | CH₃ |
| B-25 | CH₂CH₂CH₂CH₃ | CH₃ | CH₃ |
| B-26 | CH₂CH₂CH₂CH₃ | CH₂CH₃ | CH₃ |
| B-27 | CH₂CH₂CH₂CH₃ | CH₂CH₂CH₃ | CH₃ |
| B-28 | CH₂CH₂CH₂CH₃ | CH₂CH₂CH₂CH₃ | CH₃ |
| B-29 | (±)CH(CH₃)—CH₂CH₃ | H | CH₃ |
| B-30 | (±)CH(CH₃)—CH₂CH₃ | CH₃ | CH₃ |
| B-31 | (±)CH(CH₃)—CH₂CH₃ | CH₂CH₃ | CH₃ |
| B-32 | (S)CH(CH₃)—CH₂CH₃ | H | CH₃ |
| B-33 | (S)CH(CH₃)—CH₂CH₃ | CH₃ | CH₃ |
| B-34 | (S)CH(CH₃)—CH₂CH₃ | CH₂CH₃ | CH₃ |
| B-35 | (R)CH(CH₃)—CH₂CH₃ | H | CH₃ |
| B-36 | (R)CH(CH₃)—CH₂CH₃ | CH₃ | CH₃ |
| B-37 | (R)CH(CH₃)—CH₂CH₃ | CH₂CH₃ | CH₃ |
| B-38 | (±)CH(CH₃)—CH(CH₃)₂ | H | CH₃ |
| B-39 | (±)CH(CH₃)—CH(CH₃)₂ | CH₃ | CH₃ |
| B-40 | (±)CH(CH₃)—CH(CH₃)₂ | CH₂CH₃ | CH₃ |
| B-41 | (S)CH(CH₃)—CH(CH₃)₂ | H | CH₃ |
| B-42 | (S)CH(CH₃)—CH(CH₃)₂ | CH₃ | CH₃ |
| B-43 | (S)CH(CH₃)—CH(CH₃)₂ | CH₂CH₃ | CH₃ |
| B-44 | (R)CH(CH₃)—CH(CH₃)₂ | H | CH₃ |
| B-45 | (R)CH(CH₃)—CH(CH₃)₂ | CH₃ | CH₃ |
| B-46 | (R)CH(CH₃)—CH(CH₃)₂ | CH₂CH₃ | CH₃ |
| B-47 | (±)CH(CH₃)—C(CH₃)₃ | H | CH₃ |
| B-48 | (±)CH(CH₃)—C(CH₃)₃ | CH₃ | CH₃ |
| B-49 | (±)CH(CH₃)—C(CH₃)₃ | CH₂CH₃ | CH₃ |
| B-50 | (S)CH(CH₃)—C(CH₃)₃ | H | CH₃ |
| B-51 | (S)CH(CH₃)—C(CH₃)₃ | CH₃ | CH₃ |
| B-52 | (S)CH(CH₃)—C(CH₃)₃ | CH₂CH₃ | CH₃ |
| B-53 | (R)CH(CH₃)—C(CH₃)₃ | H | CH₃ |
| B-54 | (R)CH(CH₃)—C(CH₃)₃ | CH₃ | CH₃ |
| B-55 | (R)CH(CH₃)—C(CH₃)₃ | CH₂CH₃ | CH₃ |
| B-56 | (±)CH(CH₃)—CF₃ | H | CH₃ |
| B-57 | (±)CH(CH₃)—CF₃ | CH₃ | CH₃ |
| B-58 | (±)CH(CH₃)—CF₃ | CH₂CH₃ | CH₃ |
| B-59 | (S)CH(CH₃)—CF₃ | H | CH₃ |
| B-60 | (S)CH(CH₃)—CF₃ | CH₃ | CH₃ |
| B-61 | (S)CH(CH₃)—CF₃ | CH₂CH₃ | CH₃ |
| B-62 | (R)CH(CH₃)—CF₃ | H | CH₃ |
| B-63 | (R)CH(CH₃)—CF₃ | CH₃ | CH₃ |
| B-64 | (R)CH(CH₃)—CF₃ | CH₂CH₃ | CH₃ |
| B-65 | (±)CH(CH₃)—CCl₃ | H | CH₃ |
| B-66 | (±)CH(CH₃)—CCl₃ | CH₃ | CH₃ |
| B-67 | (±)CH(CH₃)—CCl₃ | CH₂CH₃ | CH₃ |
| B-68 | (S)CH(CH₃)—CCl₃ | H | CH₃ |
| B-69 | (S)CH(CH₃)—CCl₃ | CH₃ | CH₃ |
| B-70 | (S)CH(CH₃)—CCl₃ | CH₂CH₃ | CH₃ |
| B-71 | (R)CH(CH₃)—CCl₃ | H | CH₃ |
| B-72 | (R)CH(CH₃)—CCl₃ | CH₃ | CH₃ |
| B-73 | (R)CH(CH₃)—CCl₃ | CH₂CH₃ | CH₃ |
| B-74 | CH₂CF₂CF₃ | H | CH₃ |
| B-75 | CH₂CF₂CF₃ | CH₃ | CH₃ |
| B-76 | CH₂CF₂CF₃ | CH₂CH₃ | CH₃ |
| B-77 | CH₂(CF₂)₂CF₃ | H | CH₃ |
| B-78 | CH₂(CF₂)₂CF₃ | CH₃ | CH₃ |
| B-79 | CH₂(CF₂)₂CF₃ | CH₂CH₃ | CH₃ |
| B-80 | CH₂C(CH₃)═CH₂ | H | CH₃ |
| B-81 | CH₂C(CH₃)═CH₂ | CH₃ | CH₃ |
| B-82 | CH₂C(CH₃)═CH₂ | CH₂CH₃ | CH₃ |
| B-83 | CH₂CH═CH₂ | H | CH₃ |
| B-84 | CH₂CH═CH₂ | CH₃ | CH₃ |
| B-85 | CH₂CH═CH₂ | CH₂CH₃ | CH₃ |
| B-86 | (±)CH(CH₃)CH═CH₂ | H | CH₃ |
| B-87 | (±)CH(CH₃)CH═CH₂ | CH₃ | CH₃ |
| B-88 | (±)CH(CH₃)CH═CH₂ | CH₂CH₃ | CH₃ |
| B-89 | (S)CH(CH₃)CH═CH₂ | H | CH₃ |
| B-90 | (S)CH(CH₃)CH═CH₂ | CH₃ | CH₃ |
| B-91 | (S)CH(CH₃)CH═CH₂ | CH₂CH₃ | CH₃ |
| B-92 | (R)CH(CH₃)CH═CH₂ | H | CH₃ |
| B-93 | (R)CH(CH₃)CH═CH₂ | CH₃ | CH₃ |
| B-94 | (R)CH(CH₃)CH═CH₂ | CH₂CH₃ | CH₃ |
| B-95 | CH(CH₃)C(CH₃)═CH₂ | H | CH₃ |
| B-96 | CH(CH₃)C(CH₃)═CH₂ | CH₃ | CH₃ |
| B-97 | CH(CH₃)C(CH₃)═CH₂ | CH₂CH₃ | CH₃ |
| B-98 | CH₂—C≡CH | H | CH₃ |
| B-99 | CH₂—C≡CH | CH₃ | CH₃ |
| B-100 | CH₂—C≡CH | CH₂CH₃ | CH₃ |
| B-101 | Cyclopentyl | H | CH₃ |
| B-102 | Cyclopentyl | CH₃ | CH₃ |
| B-103 | Cyclopentyl | CH₂CH₃ | CH₃ |
| B-104 | Cyclohexyl | H | CH₃ |
| B-105 | Cyclohexyl | CH₃ | CH₃ |
| B-106 | Cyclohexyl | CH₂CH₃ | CH₃ |
| B-107 | CH₂—C₆H₅ | H | CH₃ |
| B-108 | CH₂—C₆H₅ | CH₃ | CH₃ |
| B-109 | CH₂—C₆H₅ | CH₂CH₃ | CH₃ |
| B-110 | NH₂ | CH₂-c-C₆H₁₁ | CH₃ |
| B-111 | NH₂ | CH₂CH₃ | CH₃ |
| B-112 | NH₂ | CH₂CH₂CH₃ | CH₃ |
| B-113 | NH—CH₂—CH═CH₂ | H | CH₃ |
| B-114 | NH—CH₂—CH═CH₂ | CH₃ | CH₃ |
| B-115 | NH—CH₂—CH═CH₂ | CH₂CH₃ | CH₃ |
| B-116 | NH—C(CH₃)₃ | H | CH₃ |
| B-117 | N(CH₃)₂ | H | CH₃ |
| B-118 | NH(CH₃) | H | CH₃ |
| B-119 | —(CH₂)₂CH═CHCH₂— | | CH₃ |
| B-120 | —(CH₂)₂C(CH₃)═CHCH₂— | | CH₃ |
| B-121 | —CH(CH₃)CH₂—CH═CHCH₂— | | CH₃ |
| B-122 | —(CH₂)₂CH(CH₃)(CH₂)₂— | | CH₃ |
| B-123 | —(CH₂)₃CHFCH₂— | | CH₃ |
| B-124 | —(CH₂)₂CHF(CH₂)₂— | | CH₃ |
| B-125 | —CH₂CHF(CH₂)₃— | | CH₃ |
| B-126 | —(CH₂)₂CH(CF₃)(CH₂)₂— | | CH₃ |
| B-127 | —(CH₂)₂O(CH₂)₂— | | CH₃ |
| B-128 | —(CH₂)₂S(CH₂)₂— | | CH₃ |
| B-129 | —(CH₂)₅— | | CH₃ |
| B-130 | —(CH₂)₄— | | CH₃ |
| B-131 | —CH₂CH═CHCH₂— | | CH₃ |
| B-132 | —CH(CH₃)(CH₂)₃— | | CH₃ |
| B-133 | —CH₂CH(CH₃)(CH₂)₂— | | CH₃ |
| B-134 | —CH(CH₃)—(CH₂)₂—CH(CH₃)— | | CH₃ |
| B-135 | —CH(CH₃)—(CH₂)₄— | | CH₃ |
| B-136 | —CH₂—CH(CH₃)—(CH₂)₃— | | CH₃ |
| B-137 | —(CH₂)—CH(CH₃)—CH₂—CH(CH₃)—CH₂— | | CH₃ |
| B-138 | —CH(CH₂CH₃)—(CH₂)₄— | | CH₃ |
| B-139 | —(CH₂)₂—CHOH—(CH₂)₂— | | CH₃ |
| B-140 | —(CH₂)₆— | | CH₃ |
| B-141 | —CH(CH₃)—(CH₂)₅— | | CH₃ |
| B-142 | —(CH₂)₂—N(CH₃)—(CH₂)₂— | | CH₃ |
| B-143 | —N═CH—CH═CH— | | CH₃ |
| B-144 | —N═C(CH₃)—CH═C(CH₃)— | | CH₃ |
| B-145 | —N═C(CF₃)—CH═C(CF₃)— | | CH₃ |
| B-146 | H | H | C₂H₅ |
| B-147 | CH₃ | H | C₂H₅ |
| B-148 | CH₃ | CH₃ | C₂H₅ |
| B-149 | CH₂CH₃ | H | C₂H₅ |
| B-150 | CH₂CH₃ | CH₃ | C₂H₅ |
| B-151 | CH₂CH₃ | CH₂CH₃ | C₂H₅ |
| B-152 | CH₂CF₃ | H | C₂H₅ |
| B-153 | CH₂CF₃ | CH₃ | C₂H₅ |
| B-154 | CH₂CF₃ | CH₂CH₃ | C₂H₅ |
| B-155 | CH₂CCl₃ | H | C₂H₅ |
| B-156 | CH₂CCl₃ | CH₃ | C₂H₅ |
| B-157 | CH₂CCl₃ | CH₂CH₃ | C₂H₅ |
| B-158 | CH₂CH₂CH₃ | H | C₂H₅ |
| B-159 | CH₂CH₂CH₃ | CH₃ | C₂H₅ |
| B-160 | CH₂CH₂CH₃ | CH₂CH₃ | C₂H₅ |
| B-161 | CH₂CH₂CH₃ | CH₂CH₂CH₃ | C₂H₅ |
| B-162 | CH(CH₃)₂ | H | C₂H₅ |
| B-163 | CH(CH₃)₂ | CH₃ | C₂H₅ |
| B-164 | CH(CH₃)₂ | CH₂CH₃ | C₂H₅ |
| B-165 | CH₂CH(CH₃)₂ | H | C₂H₅ |
| B-166 | CH₂CH(CH₃)₂ | CH₃ | C₂H₅ |
| B-167 | CH₂CH(CH₃)₂ | CH₂CH₃ | C₂H₅ |
| B-168 | CH₂CH(CH₃)₂ | CH₂CH₂CH₃ | C₂H₅ |
| B-169 | CH₂CH₂CH₂CH₃ | H | C₂H₅ |
| B-170 | CH₂CH₂CH₂CH₃ | CH₃ | C₂H₅ |
| B-171 | CH₂CH₂CH₂CH₃ | CH₂CH₃ | C₂H₅ |
| B-172 | CH₂CH₂CH₂CH₃ | CH₂CH₂CH₃ | C₂H₅ |
| B-173 | CH₂CH₂CH₂CH₃ | CH₂CH₂CH₂CH₃ | C₂H₅ |
| B-174 | (±)CH(CH₃)—CH₂CH₃ | H | C₂H₅ |
| B-175 | (±)CH(CH₃)—CH₂CH₃ | CH₃ | C₂H₅ |
| B-176 | (±)CH(CH₃)—CH₂CH₃ | CH₂CH₃ | C₂H₅ |
| B-177 | (S)CH(CH₃)—CH₂CH₃ | H | C₂H₅ |
| B-178 | (S)CH(CH₃)—CH₂CH₃ | CH₃ | C₂H₅ |
| B-179 | (S)CH(CH₃)—CH₂CH₃ | CH₂CH₃ | C₂H₅ |
| B-180 | (R)CH(CH₃)—CH₂CH₃ | H | C₂H₅ |
| B-181 | (R)CH(CH₃)—CH₂CH₃ | CH₃ | C₂H₅ |
| B-182 | (R)CH(CH₃)—CH₂CH₃ | CH₂CH₃ | C₂H₅ |
| B-183 | (±)CH(CH₃)—CH(CH₃)₂ | H | C₂H₅ |
| B-184 | (±)CH(CH₃)—CH(CH₃)₂ | CH₃ | C₂H₅ |
| B-185 | (±)CH(CH₃)—CH(CH₃)₂ | CH₂CH₃ | C₂H₅ |
| B-186 | (S)CH(CH₃)—CH(CH₃)₂ | H | C₂H₅ |
| B-187 | (S)CH(CH₃)—CH(CH₃)₂ | CH₃ | C₂H₅ |
| B-188 | (S)CH(CH₃)—CH(CH₃)₂ | CH₂CH₃ | C₂H₅ |
| B-189 | (R)CH(CH₃)—CH(CH₃)₂ | H | C₂H₅ |
| B-190 | (R)CH(CH₃)—CH(CH₃)₂ | CH₃ | C₂H₅ |
| B-191 | (R)CH(CH₃)—CH(CH₃)₂ | CH₂CH₃ | C₂H₅ |
| B-192 | (±)CH(CH₃)—C(CH₃)₃ | H | C₂H₅ |
| B-193 | (±)CH(CH₃)—C(CH₃)₃ | CH₃ | C₂H₅ |
| B-194 | (±)CH(CH₃)—C(CH₃)₃ | CH₂CH₃ | C₂H₅ |
| B-195 | (S)CH(CH₃)—C(CH₃)₃ | H | C₂H₅ |
| B-196 | (S)CH(CH₃)—C(CH₃)₃ | CH₃ | C₂H₅ |
| B-197 | (S)CH(CH₃)—C(CH₃)₃ | CH₂CH₃ | C₂H₅ |
| B-198 | (R)CH(CH₃)—C(CH₃)₃ | H | C₂H₅ |
| B-199 | (R)CH(CH₃)—C(CH₃)₃ | CH₃ | C₂H₅ |
| B-200 | (R)CH(CH₃)—C(CH₃)₃ | CH₂CH₃ | C₂H₅ |
| B-201 | (±)CH(CH₃)—CF₃ | H | C₂H₅ |
| B-202 | (±)CH(CH₃)—CF₃ | CH₃ | C₂H₅ |
| B-203 | (±)CH(CH₃)—CF₃ | CH₂CH₃ | C₂H₅ |
| B-204 | (S)CH(CH₃)—CF₃ | H | C₂H₅ |
| B-205 | (S)CH(CH₃)—CF₃ | CH₃ | C₂H₅ |
| B-206 | (S)CH(CH₃)—CF₃ | CH₂CH₃ | C₂H₅ |
| B-207 | (R)CH(CH₃)—CF₃ | H | C₂H₅ |
| B-208 | (R)CH(CH₃)—CF₃ | CH₃ | C₂H₅ |
| B-209 | (R)CH(CH₃)—CF₃ | CH₂CH₃ | C₂H₅ |
| B-210 | (±)CH(CH₃)—CCl₃ | H | C₂H₅ |
| B-211 | (±)CH(CH₃)—CCl₃ | CH₃ | C₂H₅ |
| B-212 | (±)CH(CH₃)—CCl₃ | CH₂CH₃ | C₂H₅ |
| B-213 | (S)CH(CH₃)—CCl₃ | H | C₂H₅ |
| B-214 | (S)CH(CH₃)—CCl₃ | CH₃ | C₂H₅ |
| B-215 | (S)CH(CH₃)—CCl₃ | CH₂CH₃ | C₂H₅ |
| B-216 | (R)CH(CH₃)—CCl₃ | H | C₂H₅ |
| B-217 | (R)CH(CH₃)—CCl₃ | CH₃ | C₂H₅ |
| B-218 | (R)CH(CH₃)—CCl₃ | CH₂CH₃ | C₂H₅ |
| B-219 | CH₂CF₂CF₃ | H | C₂H₅ |
| B-220 | CH₂CF₂CF₃ | CH₃ | C₂H₅ |
| B-221 | CH₂CF₂CF₃ | CH₂CH₃ | C₂H₅ |
| B-222 | CH₂(CF₂)₂CF₃ | H | C₂H₅ |
| B-223 | CH₂(CF₂)₂CF₃ | CH₃ | C₂H₅ |
| B-224 | CH₂(CF₂)₂CF₃ | CH₂CH₃ | C₂H₅ |
| B-225 | CH₂C(CH₃)═CH₂ | H | C₂H₅ |
| B-226 | CH₂C(CH₃)═CH₂ | CH₃ | C₂H₅ |
| B-227 | CH₂C(CH₃)═CH₂ | CH₂CH₃ | C₂H₅ |
| B-228 | CH₂CH═CH₂ | H | C₂H₅ |
| B-229 | CH₂CH═CH₂ | CH₃ | C₂H₅ |
| B-230 | CH₂CH═CH₂ | CH₂CH₃ | C₂H₅ |
| B-231 | (±)CH(CH₃)CH═CH₂ | H | C₂H₅ |
| B-232 | (±)CH(CH₃)CH═CH₂ | CH₃ | C₂H₅ |
| B-233 | (±)CH(CH₃)CH═CH₂ | CH₂CH₃ | C₂H₅ |
| B-234 | (S)CH(CH₃)CH═CH₂ | H | C₂H₅ |
| B-235 | (S)CH(CH₃)CH═CH₂ | CH₃ | C₂H₅ |
| B-236 | (S)CH(CH₃)CH═CH₂ | CH₂CH₃ | C₂H₅ |
| B-237 | (R)CH(CH₃)CH═CH₂ | H | C₂H₅ |
| B-238 | (R)CH(CH₃)CH═CH₂ | CH₃ | C₂H₅ |
| B-239 | (R)CH(CH₃)CH═CH₂ | CH₂CH₃ | C₂H₅ |
| B-240 | CH(CH₃)C(CH₃)═CH₂ | H | C₂H₅ |
| B-241 | CH(CH₃)C(CH₃)═CH₂ | CH₃ | C₂H₅ |
| B-242 | CH(CH₃)C(CH₃)═CH₂ | CH₂CH₃ | C₂H₅ |
| B-243 | CH₂—C≡CH | H | C₂H₅ |
| B-244 | CH₂—C≡CH | CH₃ | C₂H₅ |
| B-245 | CH₂—C≡CH | CH₂CH₃ | C₂H₅ |
| B-246 | Cyclopentyl | H | C₂H₅ |
| B-247 | Cyclopentyl | CH₃ | C₂H₅ |
| B-248 | Cyclopentyl | CH₂CH₃ | C₂H₅ |
| B-249 | Cyclohexyl | H | C₂H₅ |
| B-250 | Cyclohexyl | CH₃ | C₂H₅ |
| B-251 | Cyclohexyl | CH₂CH₃ | C₂H₅ |
| B-252 | CH₂—C₆H₅ | H | C₂H₅ |
| B-253 | CH₂—C₆H₅ | CH₃ | C₂H₅ |
| B-254 | CH₂—C₆H₅ | CH₂CH₃ | C₂H₅ |
| B-255 | NH₂ | CH₂-c-C₆H₁₁ | C₂H₅ |
| B-256 | NH₂ | CH₂CH₃ | C₂H₅ |
| B-257 | NH₂ | CH₂CH₂CH₃ | C₂H₅ |
| B-258 | NH—CH₂—CH═CH₂ | H | C₂H₅ |
| B-259 | NH—CH₂—CH═CH₂ | CH₃ | C₂H₅ |
| B-260 | NH—CH₂—CH═CH₂ | CH₂CH₃ | C₂H₅ |
| B-261 | NH—C(CH₃)₃ | H | C₂H₅ |
| B-262 | N(CH₃)₂ | H | C₂H₅ |
| B-263 | NH(CH₃) | H | C₂H₅ |
| B-264 | —(CH₂)₂CH═CHCH₂— | | C₂H₅ |
| B-265 | —(CH₂)₂C(CH₃)═CHCH₂— | | C₂H₅ |
| B-266 | —CH(CH₃)CH₂—CH═CHCH₂— | | C₂H₅ |
| B-267 | —(CH₂)₂CH(CH₃)(CH₂)₂— | | C₂H₅ |
| B-268 | —(CH₂)₃CHFCH₂— | | C₂H₅ |
| B-269 | —(CH₂)₂CHF(CH₂)₂— | | C₂H₅ |
| B-270 | —CH₂CHF(CH₂)₃— | | C₂H₅ |
| B-271 | —(CH₂)₂CH(CF₃)(CH₂)₂— | | C₂H₅ |
| B-272 | —(CH₂)₂O(CH₂)₂— | | C₂H₅ |
| B-273 | —(CH₂)₂S(CH₂)₂— | | C₂H₅ |
| B-274 | —(CH₂)₅— | | C₂H₅ |
| B-275 | —(CH₂)₄— | | C₂H₅ |
| B-276 | —CH₂CH═CHCH₂— | | C₂H₅ |
| B-277 | —CH(CH₃)(CH₂)₃— | | C₂H₅ |
| B-278 | —CH₂CH(CH₃)(CH₂)₂— | | C₂H₅ |
| B-279 | —CH(CH₃)—(CH₂)₂—CH(CH₃)— | | C₂H₅ |
| B-280 | —CH(CH₃)—(CH₂)₄— | | C₂H₅ |
| B-281 | —CH₂—CH(CH₃)—(CH₂)₃— | | C₂H₅ |
| B-282 | —(CH₂)—CH(CH₃)—CH₂—CH(CH₃)—CH₂— | | C₂H₅ |
| B-283 | —CH(CH₂CH₃)—(CH₂)₄— | | C₂H₅ |
| B-284 | —(CH₂)₂—CHOH—(CH₂)₂— | | C₂H₅ |
| B-285 | —(CH₂)₆— | | C₂H₅ |
| B-286 | —CH(CH₃)—(CH₂)₅— | | C₂H₅ |
| B-287 | —(CH₂)₂—N(CH₃)—(CH₂)₂— | | C₂H₅ |
| B-288 | —N═CH—CH═CH— | | C₂H₅ |
| B-289 | —N═C(CH₃)—CH═C(CH₃)— | | C₂H₅ |
| B-290 | —N═C(CF₃)—CH═C(CF₃)— | | C₂H₅ |
| B-291 | H | H | (CH₂)₂CH₃ |
| B-292 | CH₃ | H | (CH₂)₂CH₃ |
| B-293 | CH₃ | CH₃ | (CH₂)₂CH₃ |
| B-294 | CH₂CH₃ | H | (CH₂)₂CH₃ |
| B-295 | CH₂CH₃ | CH₃ | (CH₂)₂CH₃ |
| B-296 | CH₂CH₃ | CH₂CH₃ | (CH₂)₂CH₃ |
| B-297 | CH₂CF₃ | H | (CH₂)₂CH₃ |
| B-298 | CH₂CF₃ | CH₃ | (CH₂)₂CH₃ |
| B-299 | CH₂CF₃ | CH₂CH₃ | (CH₂)₂CH₃ |
| B-300 | CH₂CCl₃ | H | (CH₂)₂CH₃ |
| B-301 | CH₂CCl₃ | CH₃ | (CH₂)₂CH₃ |
| B-302 | CH₂CCl₃ | CH₂CH₃ | (CH₂)₂CH₃ |
| B-303 | CH₂CH₂CH₃ | H | (CH₂)₂CH₃ |
| B-304 | CH₂CH₂CH₃ | CH₃ | (CH₂)₂CH₃ |
| B-305 | CH₂CH₂CH₃ | CH₂CH₃ | (CH₂)₂CH₃ |
| B-306 | CH₂CH₂CH₃ | CH₂CH₂CH₃ | (CH₂)₂CH₃ |
| B-307 | CH(CH₃)₂ | H | (CH₂)₂CH₃ |
| B-308 | CH(CH₃)₂ | CH₃ | (CH₂)₂CH₃ |
| B-309 | CH(CH₃)₂ | CH₂CH₃ | (CH₂)₂CH₃ |
| B-310 | CH₂CH(CH₃)₂ | H | (CH₂)₂CH₃ |
| B-311 | CH₂CH(CH₃)₂ | CH₃ | (CH₂)₂CH₃ |
| B-312 | CH₂CH(CH₃)₂ | CH₂CH₃ | (CH₂)₂CH₃ |
| B-313 | CH₂CH(CH₃)₂ | CH₂CH₂CH₃ | (CH₂)₂CH₃ |
| B-314 | CH₂CH₂CH₂CH₃ | H | (CH₂)₂CH₃ |
| B-315 | CH₂CH₂CH₂CH₃ | CH₃ | (CH₂)₂CH₃ |
| B-316 | CH₂CH₂CH₂CH₃ | CH₂CH₃ | (CH₂)₂CH₃ |
| B-317 | CH₂CH₂CH₂CH₃ | CH₂CH₂CH₃ | (CH₂)₂CH₃ |
| B-318 | CH₂CH₂CH₂CH₃ | CH₂CH₂CH₂CH₃ | (CH₂)₂CH₃ |
| B-319 | (±)CH(CH₃)—CH₂CH₃ | H | (CH₂)₂CH₃ |
| B-320 | (±)CH(CH₃)—CH₂CH₃ | CH₃ | (CH₂)₂CH₃ |
| B-321 | (±)CH(CH₃)—CH₂CH₃ | CH₂CH₃ | (CH₂)₂CH₃ |
| B-322 | (S)CH(CH₃)—CH₂CH₃ | H | (CH₂)₂CH₃ |
| B-323 | (S)CH(CH₃)—CH₂CH₃ | CH₃ | (CH₂)₂CH₃ |
| B-324 | (S)CH(CH₃)—CH₂CH₃ | CH₂CH₃ | (CH₂)₂CH₃ |
| B-325 | (R)CH(CH₃)—CH₂CH₃ | H | (CH₂)₂CH₃ |
| B-326 | (R)CH(CH₃)—CH₂CH₃ | CH₃ | (CH₂)₂CH₃ |
| B-327 | (R)CH(CH₃)—CH₂CH₃ | CH₂CH₃ | (CH₂)₂CH₃ |
| B-328 | (±)CH(CH₃)—CH(CH₃)₂ | H | (CH₂)₂CH₃ |
| B-329 | (±)CH(CH₃)—CH(CH₃)₂ | CH₃ | (CH₂)₂CH₃ |
| B-330 | (±)CH(CH₃)—CH(CH₃)₂ | CH₂CH₃ | (CH₂)₂CH₃ |
| B-331 | (S)CH(CH₃)—CH(CH₃)₂ | H | (CH₂)₂CH₃ |
| B-332 | (S)CH(CH₃)—CH(CH₃)₂ | CH₃ | (CH₂)₂CH₃ |
| B-333 | (S)CH(CH₃)—CH(CH₃)₂ | CH₂CH₃ | (CH₂)₂CH₃ |
| B-334 | (R)CH(CH₃)—CH(CH₃)₂ | H | (CH₂)₂CH₃ |
| B-335 | (R)CH(CH₃)—CH(CH₃)₂ | CH₃ | (CH₂)₂CH₃ |
| B-336 | (R)CH(CH₃)—CH(CH₃)₂ | CH₂CH₃ | (CH₂)₂CH₃ |
| B-337 | (±)CH(CH₃)—C(CH₃)₃ | H | (CH₂)₂CH₃ |
| B-338 | (±)CH(CH₃)—C(CH₃)₃ | CH₃ | (CH₂)₂CH₃ |
| B-339 | (±)CH(CH₃)—C(CH₃)₃ | CH₂CH₃ | (CH₂)₂CH₃ |
| B-340 | (S)CH(CH₃)—C(CH₃)₃ | H | (CH₂)₂CH₃ |
| B-341 | (S)CH(CH₃)—C(CH₃)₃ | CH₃ | (CH₂)₂CH₃ |
| B-342 | (S)CH(CH₃)—C(CH₃)₃ | CH₂CH₃ | (CH₂)₂CH₃ |
| B-343 | (R)CH(CH₃)—C(CH₃)₃ | H | (CH₂)₂CH₃ |
| B-344 | (R)CH(CH₃)—C(CH₃)₃ | CH₃ | (CH₂)₂CH₃ |
| B-345 | (R)CH(CH₃)—C(CH₃)₃ | CH₂CH₃ | (CH₂)₂CH₃ |
| B-346 | (±)CH(CH₃)—CF₃ | H | (CH₂)₂CH₃ |
| B-347 | (±)CH(CH₃)—CF₃ | CH₃ | (CH₂)₂CH₃ |
| B-348 | (±)CH(CH₃)—CF₃ | CH₂CH₃ | (CH₂)₂CH₃ |
| B-349 | (S)CH(CH₃)—CF₃ | H | (CH₂)₂CH₃ |
| B-350 | (S)CH(CH₃)—CF₃ | CH₃ | (CH₂)₂CH₃ |
| B-351 | (S)CH(CH₃)—CF₃ | CH₂CH₃ | (CH₂)₂CH₃ |
| B-352 | (R)CH(CH₃)—CF₃ | H | (CH₂)₂CH₃ |
| B-353 | (R)CH(CH₃)—CF₃ | CH₃ | (CH₂)₂CH₃ |
| B-354 | (R)CH(CH₃)—CF₃ | CH₂CH₃ | (CH₂)₂CH₃ |
| B-355 | (±)CH(CH₃)—CCl₃ | H | (CH₂)₂CH₃ |
| B-356 | (±)CH(CH₃)—CCl₃ | CH₃ | (CH₂)₂CH₃ |
| B-357 | (±)CH(CH₃)—CCl₃ | CH₂CH₃ | (CH₂)₂CH₃ |
| B-358 | (S)CH(CH₃)—CCl₃ | H | (CH₂)₂CH₃ |
| B-359 | (S)CH(CH₃)—CCl₃ | CH₃ | (CH₂)₂CH₃ |
| B-360 | (S)CH(CH₃)—CCl₃ | CH₂CH₃ | (CH₂)₂CH₃ |
| B-361 | (R)CH(CH₃)—CCl₃ | H | (CH₂)₂CH₃ |
| B-362 | (R)CH(CH₃)—CCl₃ | CH₃ | (CH₂)₂CH₃ |
| B-363 | (R)CH(CH₃)—CCl₃ | CH₂CH₃ | (CH₂)₂CH₃ |
| B-364 | CH₂CF₂CF₃ | H | (CH₂)₂CH₃ |
| B-365 | CH₂CF₂CF₃ | CH₃ | (CH₂)₂CH₃ |
| B-366 | CH₂CF₂CF₃ | CH₂CH₃ | (CH₂)₂CH₃ |
| B-367 | CH₂(CF₂)₂CF₃ | H | (CH₂)₂CH₃ |
| B-368 | CH₂(CF₂)₂CF₃ | CH₃ | (CH₂)₂CH₃ |
| B-369 | CH₂(CF₂)₂CF₃ | CH₂CH₃ | (CH₂)₂CH₃ |
| B-370 | CH₂C(CH₃)═CH₂ | H | (CH₂)₂CH₃ |
| B-371 | CH₂C(CH₃)═CH₂ | CH₃ | (CH₂)₂CH₃ |
| B-372 | CH₂C(CH₃)═CH₂ | CH₂CH₃ | (CH₂)₂CH₃ |
| B-373 | CH₂CH═CH₂ | H | (CH₂)₂CH₃ |
| B-374 | CH₂CH═CH₂ | CH₃ | (CH₂)₂CH₃ |
| B-375 | CH₂CH═CH₂ | CH₂CH₃ | (CH₂)₂CH₃ |
| B-376 | (±)CH(CH₃)CH═CH₂ | H | (CH₂)₂CH₃ |
| B-377 | (±)CH(CH₃)CH═CH₂ | CH₃ | (CH₂)₂CH₃ |
| B-378 | (±)CH(CH₃)CH═CH₂ | CH₂CH₃ | (CH₂)₂CH₃ |
| B-379 | (S)CH(CH₃)CH═CH₂ | H | (CH₂)₂CH₃ |
| B-380 | (S)CH(CH₃)CH═CH₂ | CH₃ | (CH₂)₂CH₃ |
| B-381 | (S)CH(CH₃)CH═CH₂ | CH₂CH₃ | (CH₂)₂CH₃ |
| B-382 | (R)CH(CH₃)CH═CH₂ | H | (CH₂)₂CH₃ |
| B-383 | (R)CH(CH₃)CH═CH₂ | CH₃ | (CH₂)₂CH₃ |
| B-384 | (R)CH(CH₃)CH═CH₂ | CH₂CH₃ | (CH₂)₂CH₃ |
| B-385 | CH(CH₃)C(CH₃)═CH₂ | H | (CH₂)₂CH₃ |
| B-386 | CH(CH₃)C(CH₃)═CH₂ | CH₃ | (CH₂)₂CH₃ |
| B-387 | CH(CH₃)C(CH₃)═CH₂ | CH₂CH₃ | (CH₂)₂CH₃ |
| B-388 | CH₂—C≡CH | H | (CH₂)₂CH₃ |
| B-389 | CH₂—C≡CH | CH₃ | (CH₂)₂CH₃ |
| B-390 | CH₂—C≡CH | CH₂CH₃ | (CH₂)₂CH₃ |
| B-391 | Cyclopentyl | H | (CH₂)₂CH₃ |
| B-392 | Cyclopentyl | CH₃ | (CH₂)₂CH₃ |
| B-393 | Cyclopentyl | CH₂CH₃ | (CH₂)₂CH₃ |
| B-394 | Cyclohexyl | H | (CH₂)₂CH₃ |
| B-395 | Cyclohexyl | CH₃ | (CH₂)₂CH₃ |
| B-396 | Cyclohexyl | CH₂CH₃ | (CH₂)₂CH₃ |
| B-397 | CH₂—C₆H₅ | H | (CH₂)₂CH₃ |
| B-398 | CH₂—C₆H₅ | CH₃ | (CH₂)₂CH₃ |
| B-399 | CH₂—C₆H₅ | CH₂CH₃ | (CH₂)₂CH₃ |
| B-400 | NH₂ | CH₂-c-C₆H₁₁ | (CH₂)₂CH₃ |
| B-401 | NH₂ | CH₂CH₃ | (CH₂)₂CH₃ |
| B-402 | NH₂ | CH₂CH₂CH₃ | (CH₂)₂CH₃ |
| B-403 | NH—CH₂—CH═CH₂ | H | (CH₂)₂CH₃ |
| B-404 | NH—CH₂—CH═CH₂ | CH₃ | (CH₂)₂CH₃ |
| B-405 | NH—CH₂—CH═CH₂ | CH₂CH₃ | (CH₂)₂CH₃ |
| B-406 | NH—C(CH₃)₃ | H | (CH₂)₂CH₃ |
| B-407 | N(CH₃)₂ | H | (CH₂)₂CH₃ |
| B-408 | NH(CH₃) | H | (CH₂)₂CH₃ |
| B-409 | —(CH₂)₂CH═CHCH₂— | | (CH₂)₂CH₃ |
| B-410 | —(CH₂)₂C(CH₃)═CHCH₂— | | (CH₂)₂CH₃ |
| B-411 | —CH(CH₃)CH₂—CH═CHCH₂— | | (CH₂)₂CH₃ |
| B-412 | —(CH₂)₂CH(CH₃)(CH₂)₂— | | (CH₂)₂CH₃ |
| B-413 | —(CH₂)₃CHFCH₂— | | (CH₂)₂CH₃ |
| B-414 | —(CH₂)₂CHF(CH₂)₂— | | (CH₂)₂CH₃ |
| B-415 | —CH₂CHF(CH₂)₃— | | (CH₂)₂CH₃ |
| B-416 | —(CH₂)₂CH(CF₃)(CH₂)₂— | | (CH₂)₂CH₃ |
| B-417 | —(CH₂)₂O(CH₂)₂— | | (CH₂)₂CH₃ |
| B-418 | —(CH₂)₂S(CH₂)₂— | | (CH₂)₂CH₃ |
| B-419 | —(CH₂)₅— | | (CH₂)₂CH₃ |
| B-420 | —(CH₂)₄— | | (CH₂)₂CH₃ |
| B-421 | —CH₂CH═CHCH₂— | | (CH₂)₂CH₃ |
| B-422 | —CH(CH₃)(CH₂)₃— | | (CH₂)₂CH₃ |
| B-423 | —CH₂CH(CH₃)(CH₂)₂— | | (CH₂)₂CH₃ |
| B-424 | —CH(CH₃)—(CH₂)₂—CH(CH₃)— | | (CH₂)₂CH₃ |
| B-425 | —CH(CH₃)—(CH₂)₄— | | (CH₂)₂CH₃ |
| B-426 | —CH₂—CH(CH₃)—(CH₂)₃— | | (CH₂)₂CH₃ |
| B-427 | —(CH₂)—CH(CH₃)—CH₂—CH(CH₃)—CH₂— | | (CH₂)₂CH₃ |
| B-428 | —CH(CH₂CH₃)—(CH₂)₄— | | (CH₂)₂CH₃ |
| B-429 | —(CH₂)₂—CHOH-(CH₂)₂— | | (CH₂)₂CH₃ |
| B-430 | —(CH₂)₆— | | (CH₂)₂CH₃ |
| B-431 | —CH(CH₃)—(CH₂)₅— | | (CH₂)₂CH₃ |
| B-432 | —(CH₂)₂—N(CH₃)—(CH₂)₂— | | (CH₂)₂CH₃ |
| B-433 | —N═CH—CH═CH— | | (CH₂)₂CH₃ |
| B-434 | —N═C(CH₃)—CH═C(CH₃)— | | (CH₂)₂CH₃ |
| B-435 | —N═C(CF₃)—CH═C(CF₃)— | | (CH₂)₂CH₃ |
| B-436 | H | H | CH₂OCH₃ |
| B-437 | CH₃ | H | CH₂OCH₃ |
| B-438 | CH₃ | CH₃ | CH₂OCH₃ |
| B-439 | CH₂CH₃ | H | CH₂OCH₃ |
| B-440 | CH₂CH₃ | CH₃ | CH₂OCH₃ |
| B-441 | CH₂CH₃ | CH₂CH₃ | CH₂OCH₃ |
| B-442 | CH₂CF₃ | H | CH₂OCH₃ |
| B-443 | CH₂CF₃ | CH₃ | CH₂OCH₃ |
| B-444 | CH₂CF₃ | CH₂CH₃ | CH₂OCH₃ |
| B-445 | CH₂CCl₃ | H | CH₂OCH₃ |
| B-446 | CH₂CCl₃ | CH₃ | CH₂OCH₃ |
| B-447 | CH₂CCl₃ | CH₂CH₃ | CH₂OCH₃ |
| B-448 | CH₂CH₂CH₃ | H | CH₂OCH₃ |
| B-449 | CH₂CH₂CH₃ | CH₃ | CH₂OCH₃ |
| B-450 | CH₂CH₂CH₃ | CH₂CH₃ | CH₂OCH₃ |
| B-451 | CH₂CH₂CH₃ | CH₂CH₂CH₃ | CH₂OCH₃ |
| B-452 | CH(CH₃)₂ | H | CH₂OCH₃ |
| B-453 | CH(CH₃)₂ | CH₃ | CH₂OCH₃ |
| B-454 | CH(CH₃)₂ | CH₂CH₃ | CH₂OCH₃ |
| B-455 | CH₂CH(CH₃)₂ | H | CH₂OCH₃ |
| B-456 | CH₂CH(CH₃)₂ | CH₃ | CH₂OCH₃ |
| B-457 | CH₂CH(CH₃)₂ | CH₂CH₃ | CH₂OCH₃ |
| B-458 | CH₂CH(CH₃)₂ | CH₂CH₂CH₃ | CH₂OCH₃ |
| B-459 | CH₂CH₂CH₂CH₃ | H | CH₂OCH₃ |
| B-460 | CH₂CH₂CH₂CH₃ | CH₃ | CH₂OCH₃ |
| B-461 | CH₂CH₂CH₂CH₃ | CH₂CH₃ | CH₂OCH₃ |
| B-462 | CH₂CH₂CH₂CH₃ | CH₂CH₂CH₃ | CH₂OCH₃ |
| B-463 | CH₂CH₂CH₂CH₃ | CH₂CH₂CH₂CH₃ | CH₂OCH₃ |
| B-464 | (±)CH(CH₃)—CH₂CH₃ | H | CH₂OCH₃ |
| B-465 | (±)CH(CH₃)—CH₂CH₃ | CH₃ | CH₂OCH₃ |
| B-466 | (±)CH(CH₃)—CH₂CH₃ | CH₂CH₃ | CH₂OCH₃ |
| B-467 | (S)CH(CH₃)—CH₂CH₃ | H | CH₂OCH₃ |
| B-468 | (S)CH(CH₃)—CH₂CH₃ | CH₃ | CH₂OCH₃ |
| B-469 | (S)CH(CH₃)—CH₂CH₃ | CH₂CH₃ | CH₂OCH₃ |
| B-470 | (R)CH(CH₃)—CH₂CH₃ | H | CH₂OCH₃ |
| B-471 | (R)CH(CH₃)—CH₂CH₃ | CH₃ | CH₂OCH₃ |
| B-472 | (R)CH(CH₃)—CH₂CH₃ | CH₂CH₃ | CH₂OCH₃ |
| B-473 | (±)CH(CH₃)—CH(CH₃)₂ | H | CH₂OCH₃ |
| B-474 | (±)CH(CH₃)—CH(CH₃)₂ | CH₃ | CH₂OCH₃ |
| B-475 | (±)CH(CH₃)—CH(CH₃)₂ | CH₂CH₃ | CH₂OCH₃ |
| B-476 | (S)CH(CH₃)—CH(CH₃)₂ | H | CH₂OCH₃ |
| B-477 | (S)CH(CH₃)—CH(CH₃)₂ | CH₃ | CH₂OCH₃ |
| B-478 | (S)CH(CH₃)—CH(CH₃)₂ | CH₂CH₃ | CH₂OCH₃ |
| B-479 | (R)CH(CH₃)—CH(CH₃)₂ | H | CH₂OCH₃ |
| B-480 | (R)CH(CH₃)—CH(CH₃)₂ | CH₃ | CH₂OCH₃ |
| B-481 | (R)CH(CH₃)—CH(CH₃)₂ | CH₂CH₃ | CH₂OCH₃ |
| B-482 | (±)CH(CH₃)—C(CH₃)₃ | H | CH₂OCH₃ |
| B-483 | (±)CH(CH₃)—C(CH₃)₃ | CH₃ | CH₂OCH₃ |
| B-484 | (±)CH(CH₃)—C(CH₃)₃ | CH₂CH₃ | CH₂OCH₃ |
| B-485 | (S)CH(CH₃)—C(CH₃)₃ | H | CH₂OCH₃ |
| B-486 | (S)CH(CH₃)—C(CH₃)₃ | CH₃ | CH₂OCH₃ |
| B-487 | (S)CH(CH₃)—C(CH₃)₃ | CH₂CH₃ | CH₂OCH₃ |
| B-488 | (R)CH(CH₃)—C(CH₃)₃ | H | CH₂OCH₃ |
| B-489 | (R)CH(CH₃)—C(CH₃)₃ | CH₃ | CH₂OCH₃ |
| B-490 | (R)CH(CH₃)—C(CH₃)₃ | CH₂CH₃ | CH₂OCH₃ |
| B-491 | (±)CH(CH₃)—CF₃ | H | CH₂OCH₃ |
| B-492 | (±)CH(CH₃)—CF₃ | CH₃ | CH₂OCH₃ |
| B-493 | (±)CH(CH₃)—CF₃ | CH₂CH₃ | CH₂OCH₃ |
| B-494 | (S)CH(CH₃)—CF₃ | H | CH₂OCH₃ |
| B-495 | (S)CH(CH₃)—CF₃ | CH₃ | CH₂OCH₃ |
| B-496 | (S)CH(CH₃)—CF₃ | CH₂CH₃ | CH₂OCH₃ |
| B-497 | (R)CH(CH₃)—CF₃ | H | CH₂OCH₃ |
| B-498 | (R)CH(CH₃)—CF₃ | CH₃ | CH₂OCH₃ |
| B-499 | (R)CH(CH₃)—CF₃ | CH₂CH₃ | CH₂OCH₃ |
| B-500 | (±)CH(CH₃)—CCl₃ | H | CH₂OCH₃ |
| B-501 | (±)CH(CH₃)—CCl₃ | CH₃ | CH₂OCH₃ |
| B-502 | (±)CH(CH₃)—CCl₃ | CH₂CH₃ | CH₂OCH₃ |
| B-503 | (S)CH(CH₃)—CCl₃ | H | CH₂OCH₃ |
| B-504 | (S)CH(CH₃)—CCl₃ | CH₃ | CH₂OCH₃ |
| B-505 | (S)CH(CH₃)—CCl₃ | CH₂CH₃ | CH₂OCH₃ |
| B-506 | (R)CH(CH₃)—CCl₃ | H | CH₂OCH₃ |
| B-507 | (R)CH(CH₃)—CCl₃ | CH₃ | CH₂OCH₃ |
| B-508 | (R)CH(CH₃)—CCl₃ | CH₂CH₃ | CH₂OCH₃ |
| B-509 | CH₂CF₂CF₃ | H | CH₂OCH₃ |
| B-510 | CH₂CF₂CF₃ | CH₃ | CH₂OCH₃ |
| B-511 | CH₂CF₂CF₃ | CH₂CH₃ | CH₂OCH₃ |
| B-512 | CH₂(CF₂)₂CF₃ | H | CH₂OCH₃ |
| B-513 | CH₂(CF₂)₂CF₃ | CH₃ | CH₂OCH₃ |
| B-514 | CH₂(CF₂)₂CF₃ | CH₂CH₃ | CH₂OCH₃ |
| B-515 | CH₂C(CH₃)═CH₂ | H | CH₂OCH₃ |
| B-516 | CH₂C(CH₃)═CH₂ | CH₃ | CH₂OCH₃ |
| B-517 | CH₂C(CH₃)═CH₂ | CH₂CH₃ | CH₂OCH₃ |
| B-518 | CH₂CH═CH₂ | H | CH₂OCH₃ |
| B-519 | CH₂CH═CH₂ | CH₃ | CH₂OCH₃ |
| B-520 | CH₂CH═CH₂ | CH₂CH₃ | CH₂OCH₃ |
| B-521 | (±)CH(CH₃)CH═CH₂ | H | CH₂OCH₃ |
| B-522 | (±)CH(CH₃)CH═CH₂ | CH₃ | CH₂OCH₃ |
| B-523 | (±)CH(CH₃)CH═CH₂ | CH₂CH₃ | CH₂OCH₃ |
| B-524 | (S)CH(CH₃)CH═CH₂ | H | CH₂OCH₃ |
| B-525 | (S)CH(CH₃)CH═CH₂ | CH₃ | CH₂OCH₃ |
| B-526 | (S)CH(CH₃)CH═CH₂ | CH₂CH₃ | CH₂OCH₃ |
| B-527 | (R)CH(CH₃)CH═CH₂ | H | CH₂OCH₃ |
| B-528 | (R)CH(CH₃)CH═CH₂ | CH₃ | CH₂OCH₃ |
| B-529 | (R)CH(CH₃)CH═CH₂ | CH₂CH₃ | CH₂OCH₃ |
| B-530 | CH(CH₃)C(CH₃)═CH₂ | H | CH₂OCH₃ |
| B-531 | CH(CH₃)C(CH₃)═CH₂ | CH₃ | CH₂OCH₃ |
| B-532 | CH(CH₃)C(CH₃)═CH₂ | CH₂CH₃ | CH₂OCH₃ |
| B-533 | CH₂—C≡CH | H | CH₂OCH₃ |
| B-534 | CH₂—C≡CH | CH₃ | CH₂OCH₃ |
| B-535 | CH₂—C≡CH | CH₂CH₃ | CH₂OCH₃ |
| B-536 | cyclopentyl | H | CH₂OCH₃ |
| B-537 | cyclopentyl | CH₃ | CH₂OCH₃ |
| B-538 | cyclopentyl | CH₂CH₃ | CH₂OCH₃ |
| B-539 | cyclohexyl | H | CH₂OCH₃ |
| B-540 | cyclohexyl | CH₃ | CH₂OCH₃ |
| B-541 | cyclohexyl | CH₂CH₃ | CH₂OCH₃ |
| B-542 | CH₂—C₆H₅ | H | CH₂OCH₃ |
| B-543 | CH₂—C₆H₅ | CH₃ | CH₂OCH₃ |
| B-544 | CH₂—C₆H₅ | CH₂CH₃ | CH₂OCH₃ |
| B-545 | NH₂ | CH₂-c-C₆H₁₁ | CH₂OCH₃ |
| B-546 | NH₂ | CH₂CH₃ | CH₂OCH₃ |
| B-547 | NH₂ | CH₂CH₂CH₃ | CH₂OCH₃ |
| B-548 | NH—CH₂—CH═CH₂ | H | CH₂OCH₃ |
| B-549 | NH—CH₂—CH═CH₂ | CH₃ | CH₂OCH₃ |
| B-550 | NH—CH₂—CH═CH₂ | CH₂CH₃ | CH₂OCH₃ |
| B-551 | NH—C(CH₃)₃ | H | CH₂OCH₃ |
| B-552 | N(CH₃)₂ | H | CH₂OCH₃ |
| B-553 | NH(CH₃) | H | CH₂OCH₃ |

| | | |
|---|---|---|
| B-554 | —(CH₂)₂CH═CHCH₂— | CH₂OCH₃ |
| B-555 | —(CH₂)₂C(CH₃)═CHCH₂— | CH₂OCH₃ |
| B-556 | —CH(CH₃)CH₂—CH═CHCH₂— | CH₂OCH₃ |
| B-557 | —(CH₂)₂CH(CH₃)(CH₂)₂— | CH₂OCH₃ |
| B-558 | —(CH₂)₃CHFCH₂— | CH₂OCH₃ |
| B-559 | —(CH₂)₂CHF(CH₂)₂— | CH₂OCH₃ |
| B-560 | —CH₂CHF(CH₂)₃— | CH₂OCH₃ |
| B-561 | —(CH₂)₂CH(CF₃)(CH₂)₂— | CH₂OCH₃ |
| B-562 | —(CH₂)₂O(CH₂)₂— | CH₂OCH₃ |
| B-563 | —(CH₂)₂S(CH₂)₂— | CH₂OCH₃ |
| B-564 | —(CH₂)₅— | CH₂OCH₃ |
| B-565 | —(CH₂)₄— | CH₂OCH₃ |
| B-566 | —CH₂CH═CHCH₂— | CH₂OCH₃ |
| B-567 | —CH(CH₃)(CH₂)₃— | CH₂OCH₃ |
| B-568 | —CH₂CH(CH₃)(CH₂)₂— | CH₂OCH₃ |
| B-569 | —CH(CH₃)—(CH₂)₂—CH(CH₃)— | CH₂OCH₃ |
| B-570 | —CH(CH₃)—(CH₂)₄— | CH₂OCH₃ |
| B-571 | —CH₂—CH(CH₃)—(CH₂)₃— | CH₂OCH₃ |
| B-572 | —(CH₂)—CH(CH₃)—CH₂—CH(CH₃)—CH₂— | CH₂OCH₃ |
| B-573 | —CH(CH₂CH₃)—(CH₂)₄— | CH₂OCH₃ |
| B-574 | —(CH₂)₂—CHOH—(CH₂)₂— | CH₂OCH₃ |
| B-575 | —(CH₂)₆— | CH₂OCH₃ |
| B-576 | —CH(CH₃)—(CH₂)₅— | CH₂OCH₃ |
| B-577 | —(CH₂)₂—N(CH₃)—(CH₂)₂— | CH₂OCH₃ |
| B-578 | —N═CH—CH═CH— | CH₂OCH₃ |
| B-579 | —N═C(CH₃)—CH═C(CH₃)— | CH₂OCH₃ |
| B-580 | —N═C(CF₃)—CH═C(CF₃)— | CH₂OCH₃ |

The compounds according to the invention and/or their agriculturally acceptable salts are suitable as active compounds, in particular as fungicides. They are distinguished by an excellent activity against a broad spectrum of phytopathogenic fungi from the class of the Ascomycetes, Deuteromycetes, Basidiomycetes and Peronosporomycetes (syn. Oomycetes). Some of them are systemically effective and can be used in crop protection as foliar fungicides, as fungicides for seed dressing and as soil fungicides.

Accordingly, the present invention furthermore provides the use of the compounds according to the invention and/or their agriculturally acceptable salts for controlling phytopathogenic fungi.

They are particularly important in the control of a multitude of fungi on various cultivated plants, such as wheat, rye, barley, oats, rice, corn, grass, bananas, cotton, soya, coffee, sugar cane, vines, fruits and ornamental plants, and vegetables, such as cucumbers, beans, tomatoes, potatoes and cucurbits, and on the seeds of these plants.

They are especially suitable for controlling the following plant diseases:
Alternaria species on vegetables, oilseed rape, sugar beet and fruit and rice, such as, for example, A. solani or A. alternata on potatoes and tomatoes;
Aphanomyces species on sugar beet and vegetables;
Ascochyta species on cereals and vegetables;
Bipolaris and Drechslera species on corn, cereals, rice and lawns, such as, for example, D. maydis on corn;
Blumeria graminis (powdery mildew) on cereals;
Botrytis cinerea (gray mold) on strawberries, vegetables, flowers and grapevines;
Bremia lactucae on lettuce;
Cercospora species on corn, soybeans, rice and sugar beet;
Cochliobolus species on corn, cereals, rice, such as, for example Cochliobolus sativus on cereals, Cochliobolus miyabeanus on rice;
Colletotricum species on soybeans and cotton;
Drechslera species, Pyrenophora species on corn, cereals, rice and lawns, such as, for example, D. teres on barley or D. tritici-repentis on wheat;
Esca on grapevines, caused by Phaeoacremonium chlamydosporium, Ph. Aleophilum and Formitipora punctata (syn. Phellinus punctatus);
Exserohilum species on corn;
Erysiphe cichoracearum and Sphaerotheca fuliginea on cucumbers;
Fusarium and Verticillium species on various plants, such as, for example, F graminearum or F. culmorum on cereals or F. oxysporum on a multitude of plants, such as, for example, tomatoes;
Gaeumanomyces graminis on cereals;
Gibberella species on cereals and rice (for example Gibberella fujikuroi on rice);
Grainstaining complex on rice;
Helminthosporium species on corn and rice;
Michrodochium nivale on cereals;
Mycosphaerella species on cereals, bananas and groundnuts, such as, for example, M. graminicola on wheat or M. fijiensis on bananas;
Peronospora species on cabbage and bulbous plants, such as, for example, P. brassicae on cabbage or P. destructor on onions;
Phakopsara pachyrhizi and Phakopsara meibomiae on soybeans;
Phomopsis species on soybeans and sunflowers;
Phytophthora infestans on potatoes and tomatoes;
Phytophthora species on various plants, such as, for example, P. capsici on bell pepper;
Plasmopara viticola on grapevines;
Podosphaera leucotricha on apples;
Pseudocercosporella herpotrichoides on cereals;
Pseudoperonospora on various plants, such as, for example, P. cubensis on cucumber or P. humili on hops;
Puccinia species on various plants, such as, for example, P. triticina, P. striformins, P. hordei or P. graminis on cereals or P. asparagi on asparagus;
Pyricularia oryzae, Corticium sasakii, Sarocladium oryzae, S. attenuatum, Entyloma oryzae on rice;
Pyricularia grisea on lawns and cereals;
Pythium spp. on lawns, rice, corn, cotton, oilseed rape, sunflowers, sugar beet, vegetables and other plants, such as, for example, P. ultiumum on various plants, P. aphanidermatum on lawns;
Rhizoctonia species on cotton, rice, potatoes, lawns, corn, oilseed rape, potatoes, sugar beet, vegetables and on various plants, such as, for example, R. solani on beet and various plants;
Rhynchosporium secalis on barley, rye and triticale;
Sclerotinia species on oilseed rape and sunflowers;
Septoria tritici and Stagonospora nodorum on wheat;
Erysiphe (syn. Uncinula) necator on grapevines;
Setospaeria species on corn and lawns;
Sphacelotheca reilinia on corn;
Thievaliopsis species on soybeans and cotton;
Tilletia species on cereals;
Ustilago species on cereals, corn and sugar cane, such as, for example, U. maydis on corn;
Venturia species (scab) on apples and pears, such as, for example, V. inaequalis on apples.

In addition, the compounds according to the invention can also be used in crops which, owing to breeding including genetic engineering, are tolerant to attack by insects or fungi.

The compounds according to the invention and/or their agriculturally acceptable salts are also suitable for controlling harmful fungi in the protection of materials (for example wood, paper, paint dispersions, fibers or fabrics) and in the protection of stored products. In the protection of wood, particular attention is paid to the following harmful fungi: Ascomycetes, such as Ophiostoma spp., Ceratocystis spp., Aureobasidium pullulans, Sclerophoma spp., Chaetomium spp., Humicola spp., Petriella spp., Trichurus spp.; Basidiomycetes, such as Coniophora spp., Coriolus spp., Gloeophyllum spp., Lentinus spp., Pleurotus spp., Poria spp., Serpula spp. and Tyromyces spp., Deuteromycetes, such as Aspergillus spp., Cladosporium spp., Penicillium spp., Trichoderma spp., Alternaria spp., Paecilomyces spp. and Zygomycetes, such as Mucor spp., additionally in the protection of materials the following yeasts: Candida spp. and Saccharomyces cerevisae.

The compounds according to the invention and/or their agriculturally acceptable salts are employed by treating the fungi or the plants, seeds, materials or the soil to be protected against fungal attack with a fungicidally effective amount of the active compounds. Application can be both before and after the infection of the materials, plants or seeds by the fungi.

Accordingly, the present invention furthermore provides a process for controlling phytopathogenic fungi which comprises treating the fungi or the materials, plants, the soil or seeds to be protected against fungal attack with an effective amount of at least one compound according to the invention and/or an agriculturally acceptable salt thereof.

The present invention furthermore provides a composition for controlling phytopathogenic fungi, which composition comprises at least one compound according to the invention and/or an agriculturally acceptable salt thereof and at least one solid or liquid carrier.

The fungicidal compositions generally comprise between 0.1 and 95% by weight, preferably between 0.5 and 90% by weight, of active compound.

When employed in crop protection, the amounts applied are, depending on the kind of effect desired, between 0.01 and 2.0 kg of active compound per ha.

In seed treatment, the amounts of active compound used are generally from 1 to 1000 g/100 kg of seed, preferably from 1 to 200 g/100 kg of seed, in particular from 5 to 100 g/100 kg of seed.

Accordingly, the present invention furthermore provides seed comprising a compound according to the invention in an amount of from 1 to 1000 g per 100 kg.

The present invention furthermore provides a composition for controlling phytopathogenic fungi, which composition comprises at least one compound according to the invention and/or an agriculturally acceptable salt thereof and at least one solid or liquid carrier.

When used in the protection of materials or stored products, the amount of active compound applied depends on the kind of application area and on the desired effect. Amounts typically applied in the protection of materials are, for example, from 0.001 g to 2 kg, preferably from 0.005 g to 1 kg, of active compound per cubic meter of treated material.

The compounds according to the invention and/or their agriculturally acceptable salts can be converted into the customary formulations, for example solutions, emulsions, suspensions, dusts, powders, pastes and granules. The application form depends on the particular purpose; in each case, it should ensure a fine and uniform distribution of the compound according to the invention.

The formulations are prepared in a known manner, for example by extending the active compound with solvents and/or carriers, if appropriate using emulsifiers and dispersants. Solvents/auxiliaries which are suitable are essentially:
water, aromatic solvents (for example Solvesso products, xylene), paraffins (for example mineral oil fractions), alcohols (for example methanol, butanol, pentanol, benzyl alcohol), ketones (for example cyclohexanone, gamma-butyrolactone), pyrrolidones (NMP, NOP), acetates (glycol diacetate), glycols, fatty acid dimethylamides, fatty acids and fatty acid esters. In principle, solvent mixtures may also be used,
carriers such as ground natural minerals (for example kaolins, clays, talc, chalk) and ground synthetic minerals (for example highly disperse silica, silicates); emulsifiers such as nonionic and anionic emulsifiers (for example polyoxyethylene fatty alcohol ethers, alkylsulfonates and arylsulfonates) and dispersants such as lignosulfite waste liquors and methylcellulose.

Suitable surfactants are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenol ether, ethoxylated isooctylphenol, octylphenol, nonylphenol, alkylphenol polyglycol ethers, tributylphenyl polyglycol ether, tristearylphenyl polyglycol ether, alkylaryl polyether alcohols, alcohol and fatty alcohol/ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, strongly polar solvents, for example dimethyl sulfoxide, N-methylpyrrolidone and water.

Powders, materials for spreading and dustable products can be prepared by mixing or concomitantly grinding the active substances with a solid carrier.

Granules, for example coated granules, impregnated granules and homogeneous granules, can be prepared by binding the active compounds to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the active compound. The active compounds are employed in a purity of from 90% to 100%, preferably 95% to 100% (according to NMR spectrum).

The following are examples of formulations: 1. Products for dilution with water

### A Water-Soluble Concentrates (SL, LS)

10 parts by weight of the active compounds are dissolved with 90 parts by weight of water or with a water-soluble solvent. As an alternative, wetters or other auxiliaries are added. The active compound dissolves upon dilution with water. This gives a formulation having an active compound content of 10% by weight.

### B Dispersible Concentrates (DC)

20 parts by weight of the active compounds are dissolved in 70 parts by weight of cyclohexanone with addition of 10 parts by weight of a dispersant, for example polyvinylpyrrolidone. Dilution with water gives a dispersion. The active compound content is 20% by weight

### C Emulsifiable Concentrates (EC)

15 parts by weight of the active compounds are dissolved in 75 parts by weight of xylene with addition of calcium dodecylbenzenesulfonate and castor oil ethoxylate (in each case 5 parts by weight). Dilution with water gives an emulsion. The formulation has an active compound content of 15% by weight.

### D Emulsions (EW, EO, ES)

25 parts by weight of the active compounds are dissolved in 35 parts by weight of xylene with addition of calcium dodecylbenzenesulfonate and castor oil ethoxylate (in each case 5 parts by weight). This mixture is added to 30 parts by weight of water by means of an emulsifying machine (e.g. Ultraturrax) and made into a homogeneous emulsion. Dilution with water gives an emulsion. The formulation has an active compound content of 25% by weight.

### E Suspensions (SC, OD, FS)

In an agitated ball mill, 20 parts by weight of the active compounds are comminuted with addition of 10 parts by weight of dispersants and wetters and 70 parts by weight of water or an organic solvent to give a fine active compound suspension. Dilution with water gives a stable suspension of the active compound. The active compound content in the formulation is 20% by weight.

### F Water-Dispersible Granules and Water-Soluble Granules (WG, SG)

50 parts by weight of the active compounds are ground finely with addition of 50 parts by weight of dispersants and wetters and made into water-dispersible or water-soluble granules by means of technical appliances (for example extrusion, spray tower, fluidized bed). Dilution with water gives a stable dispersion or solution of the active compound. The formulation has an active compound content of 50% by weight.

### G Water-Dispersible Powders and Water-Soluble Powders (WP, SP, SS, WS)

75 parts by weight of the active compounds are ground in a rotor-stator mill with addition of 25 parts by weight of dispersants, wetters and silica gel. Dilution with water gives a stable dispersion or solution of the active compound. The active compound content of the formulation is 75% by weight.

### H Gel Formulations (GF)

In a ball mill, 20 parts by weight of the active compounds, 10 parts by weight of dispersant, 1 part by weight of gelling agent and 70 parts by weight of water or an organic solvent are ground to give a fine suspension. On dilution with water, a stable suspension having an active compound content of 20% by weight is obtained.

### 2. Products to be Applied Undiluted

### I Dusts (DP, DS)

5 parts by weight of the active compounds are ground finely and mixed intimately with 95 parts by weight of finely divided kaolin. This gives a dustable product with an active compound content of 5% by weight.

### J Granules (GR, FG, GG, MG)

0.5 part by weight of the active compounds is ground finely and associated with 99.5 parts by weight of carriers. Current methods are extrusion, spray-drying or the fluidized bed. This gives granules with an active compound content of 0.5% by weight to be applied undiluted.

### K ULV Solutions (UL)

10 parts by weight of the active compounds are dissolved in 90 parts by weight of an organic solvent, for example xylene. This gives a product with an active compound content of 10% by weight to be applied undiluted.

For seed treatment, use is usually made of water-soluble concentrates (LS), suspensions (FS), dustable powders (DS), water-dispersible and water-soluble powders (WS, SS), emulsions (ES), emulsifiable concentrates (EC) and gel formulations (GF). These formulations can be applied to the seed in undiluted form or, preferably, diluted. Application can be carried out prior to sowing.

The active compounds can be used as such, in the form of their formulations or the use forms prepared therefrom, for example in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; the intention is to ensure in each case the finest possible distribution of the active compounds according to the invention.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetting agent, tackifier, dispersant or emulsifier. Alternatively, it is also possible to prepare concentrates composed of active substance, wetter, tackifier, dispersant or emulsifier and, if appropriate, solvent or oil, and such concentrates are suitable for dilution with water.

The active compound concentrations in the ready-to-use preparations can be varied within relatively wide ranges. In general, they are from 0.0001 to 10%, preferably from 0.01 to 1%.

The active compounds may also be used successfully in the ultra-low-volume process (ULV), by which it is possible to apply formulations comprising over 95% by weight of active compound, or even to apply the active compound without additives.

Various types of oils, wetters, adjuvants, herbicides, fungicides, other pesticides, or bactericides may be added to the active compounds, if appropriate not until immediately prior to use (tank mix). These compositions can be admixed with the compositions according to the invention in a weight ratio of from 1:100 to 100:1, preferably from 1:10 to 10:1.

Suitable adjuvants in this sense are in particular: organically modified polysiloxanes, for example Break Thru S 240®; alcohol alkoxylates, for example Atplus 2459, Atplus MBA 13030, Plurafac LF 300®5 and Lutensole ON 30; EO/PO block polymers, for example PluronicO RPE 2035 and Genapol® B; alcohol ethoxylates, for example Lutensol XP 80; and sodium dioctylsulfosuccinate, for example Leophen® RA.

The compounds according to the invention in the application form as fungicides can also be present together with other active compounds, for example with herbicides, insecticides, growth regulators, fungicides or else with fertilizers. When mixing the compounds according to the invention or the compositions comprising them with one or more further active compounds, in particular fungicides, it is in many cases possible, for example, to widen the activity spectrum or to prevent the development of resistance. In many cases, synergistic effects are obtained.

Accordingly, the present invention furthermore provides a combination of at least one compound according to the invention and/or an agriculturally acceptable salt thereof and at least one further fungicidal, insecticidal, herbicidal and/or growth-regulating active compound.

The following list of fungicides, together with which the compounds according to the invention may be used, is meant to illustrate the combination possibilities, but not to limit them:

### Strobilurins

azoxystrobin, dimoxystrobin, enestroburin, fluoxastrobin, kresoxim-methyl, metominostrobin, picoxystrobin, pyraclostrobin, trifloxystrobin, orysastrobin, methyl (2-chloro-5-[1-(3-methylbenzyloxyimino)ethyl]benzyl)carbamate, methyl (2-chloro-5-[1-(6-methylpyridin-2-ylmethoxyimino)ethyl]benzyl)carbamate, methyl 2-(ortho-((2,5-dimethylphenyloxymethylene)phenyl)-3-methoxyacrylate;

### Carboxamides

carboxanilides: benalaxyl, benodanil, boscalid, carboxin, mepronil, fenfuram, fenhexamid, flutolanil, furametpyr, metalaxyl, ofurace, oxadixyl, oxycarboxin, penthiopyrad, thifluzamide, tiadinil, N-(4′-bromobiphenyl-2-yl)-4-difluoromethyl-2-methylthiazole-5-carboxamide, N-(4′-trifluoromethylbiphenyl-2-yl)-4-difluoromethyl-2-methylthiazole-5-carboxamide, N-(4′-chloro-3′-fluorobiphenyl-2-yl)-4-difluoromethyl-2-methylthiazole-5-carboxamide, N-(3′,4′-dichloro-4-fluoro-biphenyl-2-yl)-3-difluoromethyl-1-methylpyrazole-4-carboxamide, N′-(3′,4′-dichloro-5-fluorobiphenyl-2-yl)-3-difluoromethyl-1-methylpyrazole-4-carboxamide, N-(2-cyanophenyl)-3,4-dichloroisothiazole-5-carboxamide;
carboxylic acid morpholides: dimethomorph, flumorph;
benzamides: flumetover, fluopicolide (picobenzamid), zoxamide;
other carboxamides: carpropamid, diclocymet, mandipropamid, N-(2-(4-[3-(4-chlorophenyl)prop-2-ynyloxy]-3-methoxyphenyl)ethyl)-2-methanesulfonylamino-3-methylbutyramide, N-(2-(4-[3-(4-chlorophenyl)prop-2-ynyloxy]-3-methoxyphenyl)-ethyl)-2-ethanesulfonylamino-3-methylbutyramide;

### Azoles

triazoles: bitertanol, bromuconazole, cyproconazole, difenoconazole, diniconazole, enilconazole, epoxiconazole, fenbuconazole, flusilazole, fluquinconazole, flutriafol, hexaconazole, imibenconazole, ipconazole, metconazole, myclobutanil, penconazole, propiconazole, prothioconazole, simeconazole, tebuconazole, tetraconazole, triadimenol, triadimefon, triticonazole;
imidazoles: cyazofamid, imazalil, pefurazoate, prochloraz, triflumizole;
benzimidazoles: benomyl, carbendazim, fuberidazole, thiabendazole;
others: ethaboxam, etridiazole, hymexazole;
   Nitrogenous heterocyclyl compounds
pyridines: fluazinam, pyrifenox, 3-[5-(4-chlorophenyl)-2,3-dimethylisoxazolidin-3-yl]pyridine;
pyrimidines: bupirimate, cyprodinil, ferimzone, fenarimol, mepanipyrim, nuarimol,
pyrimethanil;
piperazines: triforine;
pyrroles: fludioxonil, fenpiclonil;
morpholines: aldimorph, dodemorph, fenpropimorph, tridemorph;
dicarboximides: iprodione, procymidone, vinclozolin;
others: acibenzolar-5-methyl, anilazine, captan, captafol, dazomet, diclomezine, fenoxanil, folpet, fenpropidin, famoxadone, fenamidone, octhilinone, probenazole, proquinazid, pyroquilon, quinoxyfen, tricyclazole, 5-chloro-7-(4-methylpiperidin-1-yl)-6-(2,4,6-trifluorophenyl)-[1,2,4]triazolo[1,5-a]pyrimidine, 2-butoxy-6-iodo-3-propylchromen-4-one, N,N-dimethyl-3-(3-bromo-6-fluoro-2-methylindole-1-sulfonyl)-[1,2,4]triazole-1-sulfonamide;

### Carbamates and Dithiocarbamates

dithiocarbamates: ferbam, mancozeb, maneb, metiram, metam, propineb, thiram, zineb, ziram;
carbamates: diethofencarb, flubenthiavalicarb, iprovalicarb, propamocarb, methyl 3-(4-chlorophenyl)-3-(2-isopropoxycarbonylamino-3-methylbutyrylamino)pro-pionate, 4-fluorophenyl N-(1-(1-(4-cyanophenyl)ethanesulfonyl)but-2-yl)carbamate;
   Other fungicides
guanidines: dodine, iminoctadine, guazatine;
antibiotics: kasugamycin, polyoxins, streptomycin, validamycin A;
organometal compounds: fentin salts;
sulfur-containing heterocyclyl compounds: isoprothiolane, dithianon;
organophosphorus compounds: edifenphos, fosetyl, fosetyl-aluminum, iprobenfos, pyrazophos, tolclofos-methyl, phosphorous acid and its salts;
organochlorine compounds: thiophanate methyl, chlorothalonil, dichlofluanid, tolylfluanid, flusulfamide, phthalide, hexachlorobenzene, pencycuron, quintozene;
nitrophenyl derivatives: binapacryl, dinocap, dinobuton;
inorganic active compounds: Bordeaux mixture, copper acetate, copper hydroxide, copper oxychloride, basic copper sulfate, sulfur;
others: spiroxamine, cyflufenamid, cymoxanil, metrafenone.

The present invention furthermore relates to the pharmaceutical use of the compounds according to the invention and/or the pharmaceutically acceptable salts thereof, in particular their use for controlling tumors in mammals such as, for example, humans. Table C lists particularly preferred compounds of the present invention:

TABLE C

| | |
|---|---|
| | (I) |
| 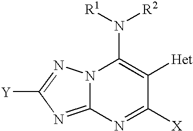 | |

| | | | | | | |
|---|---|---|---|---|---|---|
| No. | Het | R¹ | R² | X | Y | Physical data |

| | | | | | |
|---|---|---|---|---|---|
| C-1 | 3,5-dimethylpyrazol-1-yl | (CH₂)₂CH(CH₃)(CH₂)₂ | CH₃ | H | m.p. |
| | | | | | 141-142° C. |
| C-2 | 2,5-dichlorothien-3-yl | (CH₂)₂CH(CH₃)(CH₂)₂ | CH₃ | H | m.p. |
| | | | | | 106-108° C. |

### SYNTHESIS EXAMPLES

With appropriate modification of the starting materials, the procedures given in the synthesis examples below were used to obtain further compounds of the invention or precursors thereof:

### EXAMPLE 1

### 6-(3,5-Dimethylpyrazol-1-yl)-5-methyl-7-(4-methylpiperidin-1-yl)-[1,2,4]-triazolo[1,5-a]pyrimidin (C-1)

a) Diethyl 2-(3,5-dimethylpyrazol-1-yl)malonate

100 g (1040 mmol) of dimethylpyrazole were initially charged in acetonitrile, and 116 g (1144 mmol) of triethylamine and then 273.5 g (1144 mmol) of diethyl bromomalonate were added dropwise. The solution was stirred under reflux for 12 h. After cooling, the solid was filtered off, the filtrate was concentrated and the residue was extracted with water and ethyl acetate. The combined organic phases were dried over MgSO₄ and concentrated. The crude product was purified on silica gel (cyclohexane/ethyl acetate 9/1 to 1/1). This gave 210 g of the desired product.

b) 6-(3,5-Dimethylpyrazol-1-yl)-[1,2,41-triazolof 1,5-a]pyrimidine-5,7-diol

10 g (39 mmol) of the pyrazolemalonic ester and 3.5 g (41 mmol) of amitrol in 7.3 g (39 mmol) of tributylamine were stirred at 160° C. for 6 h. The ethanol formed was distilled off. About 2.5 equivalents of NaOH were dissolved in 40 ml of water and added to the cooled reaction mixture. After a further 30 min of stirring, the phases were separated and the aqueous phase was washed with ethyl acetate. The aqueous phase was then acidified with hydrochloric acid and concentrated. This gave 15.8 g of the crude product which was used directly for the next step.

c) 5,7-Dichloro-6-(3,5-dimethyipyrazol-1-yl)-[1,2,4]-triazolo[1,5-a]pyrimidine

The product from step b) and 4.9 g (51 mmol) of trimethylamine hydrochloride were stirred at reflux in 50 ml of POCl₃ for 6 h. The reaction mixture was carefully added to ice-water, neutralized with 50% strength NaOH and extracted with ethyl acetate. The combined organic phases were dried and concentrated. This gave 1.5 g of the dichloride, which was reacted further without purification.

d) 5-Chloro-6-(3,5-dimethylpyrazol-1-yl)-7-(4-methylpiperidin-1-yl)-[1 μl-4-triazolor 15-a]pyrimidine

1.5 g (5 mmol) of the product from c) were initially charged in 30 ml of dichloromethane, and 0.5 g (5 mmol) of triethylamine and 0.5 g (5 mmol) of 4-methylpiperidine were added dropwise with ice-cooling. The mixture was stirred at RT for 12 h. The mixture was washed with water and NaHCO₃ solution, and the organic phase was dried over MgSO₄ and concentrated. The crude product was stirred with cyclohexane, and the solid formed was filtered off with suction. This gave 1.2 g of the desired product.

e) Diethyl 2-[6-(3,5-dimethylpyrazol-1-yl)-7-(4-methylpiperidin-1-yl)-[1,2,4]-triazolo[1,5-a]pyrimidin-5-yl]malonate

3.5 g (26 mmol) of dimethyl malonate were initially charged in a flask. With cooling, 0.06 g (1.5 mmol) of NaH in 3 ml of diethylene glycol dimethyl ether was then added. The product from d) was suspended in 3 ml of diethylene glycol dimethyl ether and likewise added. The mixture was stirred at 50° C. for 7 h. Water was added with ice-cooling, and the mixture was extracted with ethyl acetate. The combined organic phases were dried and concentrated. The crude product was purified on silica gel (cyclohexane/ethyl acetate 1/1). This gave 0.2 g of the desired product.

f) 6-(3,5-Dimethylpyrazol-1-yl)-5-methyl-7-(4-methylpiperidin-1-yl)-[1,2,41-triazolof 1,5-a]pyrimidine

0.17 g (0.4 mmol) of the product from e) was stirred at 80° C. in 2.5 ml of conc. HCl for 4 h, and a further 12 h at RT. The solution was adjusted to pH 7. The solid formed was filtered off with suction. 0.097 g of the desired product was isolated.

### Examples of the Action of the Compounds According to the Invention Against Harmful Fungi:

### A Greenhouse Tests

The active compounds were prepared separately as a stock solution with 25 mg of active compound which was made up to 10 ml with a mixture of acetone and/or DMSO and the emulsifier Uniperol® EL (wetting agent having emulsifying and dispersing action based on ethoxylated alkylphenols) in a solvent/emulsifier volume ratio of 99 to 1. The solution was then made up to 100 ml with water. This stock solution was diluted to the active compound concentration stated below using the solvent/emulsifier/water mixture described.

Example No. 1—Curative activity against brown rust of wheat caused by Puccinia recondita

Leaves of potted wheat seedlings of the cultivar “Kanzler” were inoculated with a spore suspension of brown rust (Puccinia recondita). The pots were then placed in a chamber with high atmospheric humidity (90 to 95%) and 20 to 22° C. for 24 hours. During this time, the spores germinated and the germ tubes penetrated into the leaf tissue. The next day, the infected plants were sprayed to runoff point with the above-described active compound solution at the active compound concentration stated below. After the spray coating had dried on, the test plants were cultivated in a greenhouse at temperatures between 20 and 22° C. and at 65 to 70% relative atmospheric humidity for 7 days. The extent of the rust fungus development on the leaves was then determined.

Compound C-1 led, at 250 ppm, to an infection of 10%, whereas the untreated control was 90% infected.

### B Microtiter Test

The active compounds were formulated separately as a stock solution having a concentration of 10 000 ppm in DMSO.

Example No. 2—Activity against the rice blast pathogen Pyricularia oryzae in the microtiter test

The stock solution was pipetted onto a microtiter plate (MTP) and diluted to the stated active compound concentrate using a malt-based aqueous nutrient medium for fungi. An aqueous spore suspension of Pyricularia oryzae was then added. The plates were placed in a water vapor-saturated chamber at temperatures of 18° C. Using an absorption photometer, the MTPs were measured at 405 nm on day 7 after the inoculation. The measured parameters were compared to the growth of the active compound-free control variant (=100%) and the fungus- and active compound-free blank value to determine the relative growth in % of the pathogens in the individual active compounds.

At a concentration of 125 ppm, the compounds C-1 and C-2 led to a relative growth of 0%.

Example No. 3—Activity against the speckled leaf blotch pathogen Septoria tritici in the microtiter test

The stock solution was pipetted onto a microtiter plate (MTP) and diluted to the stated active compound concentrate using a malt-based aqueous nutrient medium for fungi. An aqueous spore suspension of Septoria tritici was then added. The plates were placed in a water vapor-saturated chamber at temperatures of 18° C. Using an absorption photometer, the MTPs were measured at 405 nm on day 7 after the inoculation. The measured parameters were compared to the growth of the active compound-free control variant (=100%) and the fungus- and active compound-free blank value to determine the relative growth in % of the pathogens in the individual active compounds.

At a concentration of 125 ppm, the compounds C-1 and C-2 led to a relative growth of at most 9%.

1.-20. (canceled)

21. A 7-amino-6-heteroaryl-1,2,4-triazolo[1,5-a]pyrimidine compound of the formula (I) 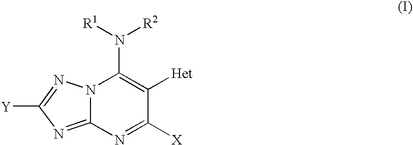
wherein,
Het is a five-membered aromatic heterocycle containing one, two, three or four heteroatoms from the group consisting of oxygen, nitrogen and sulfur, wherein Het is unsubstituted or substituted by one, two, three or four identical or different substituents L:
L is halogen, cyano, nitro, hydroxyl, cyanato (OCN), (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₄-C₁₀)-alkadienyl, (C₄-C₁₀)-haloalkadienyl, (C₁-C₈)-alkoxy, (C₁-C₈)-haloalkoxy, (C₂-C₈)-alkenyloxy, (C₂-C₈)-haloalkenyloxy, (C₂-C₈)-alkynyloxy, (C₂-C₈)-haloalkynyloxy, (C₃-C₈)-cycloalkyl, (C₃-C₈)-halocycloalkyl, (C₃-C₈)-cycloalkenyl, (C₃-C₈)-halocycloalkenyl, (C₃-C₈)-cycloalkoxy, (C₁-C₈)-alkoximino-(C₁-C₈)-alkyl, (C₂-C₈)-alkenyloximino-(C₁-C₈)-alkyl, (C₂-C₈)-alkynyloximino-(C₁-C₈)-alkyl, S(═O)ₙA¹, C(═O)A², C(═S)A², NR⁵R⁶, or NR⁵—(C═O)—R⁶; wherein
n is 0, 1 or 2;
A¹ is hydrogen, hydroxyl, (C₁-C₈)-alkyl, (C₁-C₈)-haloalkyl, amino, (C₁-C₈)-alkylamino or di-(C₁-C₈)-alkylamino,
A² is one of the groups mentioned under A¹ or (C₂-C₈)-alkenyl, (C₂-C₈)-haloalkenyl, (C₂-C₈)-alkynyl, (C₂-C₈)-haloalkynyl, (C₁-C₈)-alkoxy, (C₁-C₈)-haloalkoxy, (C₂-C₈)-alkenyloxy, (C₂-C₈)-haloalkenyloxy, (C₂-C₈)-alkynyloxy, (C₂-C₈)-haloalkynyloxy, (C₃-C₈)-cycloalkoxy, or (C₃-C₈)-halocycloalkoxy;
R⁵, R⁶ independently of one another are hydrogen, (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₃-C₈)-cycloalkyl, (C₃-C₈)-halocycloalkyl, (C₃-C₈)-cycloalkenyl or (C₃-C₈)-halocycloalkenyl,
wherein the aliphatic groups of the radicals of L may carry one to four identical or different groups R^{L};
R^{L} is halogen, cyano, (C₁-C₈)-alkoxy, (C₂-C₈)-alkenyloxy, (C₂-C₈)-alkynyloxy, (C₃-C₈)-cycloalkyl, (C₃-C₈)-cycloalkenyl, (C₃-C₈)-cycloalkyloxy, (C₃-C₈)-cycloalkenyloxy, (C₁-C₈)-alkoximino-(C₁-C₈)-alkyl, (C₂-C₈)-alkenyloximino-(C₁-C₈)-alkyl, (C₂-C₈)-alkynyloximino-(C₁-C₈)-alkyl, S(═O)ₙA¹, C(═O)A², C(═S)A², NR⁵R⁶, or NR⁵—(C—O)—R⁶;
R¹, R² independently of one another are hydrogen, (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₄-C₁₀)-alka dienyl, (C₄-C₁₀)-haloalkadienyl, (C₁-C₈)-alkoxy, (C₁-C₈)-haloalkoxy, (C₂-C₈)-alkenyloxy, (C₂-C₈)-haloalkenyloxy, (C₂-C₈)-alkynyloxy, (C₂-C₈)-haloalkynyloxy, (C₃-C₈)-cycloalkyl, (C₃-C₈)-halocycloalkyl, (C₃-C₈)-cycloalkenyl, (C₃-C₈)-halocycloalkenyl, (C₃-C₈)-cycloalkoxy, (C₃-C₈)-halocycloalkoxy, (C₅-C₁₀)-bicycloalkyl, amino, (C₁-C₈)-alkylamino, di-(C₁-C₈)-alkylamino, phenyl, naphthyl or a five- or six-membered saturated, partially unsaturated or aromatic heterocycle which contains one, two, three or four heteroatoms from the group consisting of O, N and S;
R¹ and R² together with the nitrogen atom to which they are attached may also form a five- or six-membered saturated, partially unsaturated or aromatic heterocycle which may contain one, two or three further heteroatoms from the group consisting of O, N and S as ring members, wherein
R¹ and/or R² or a heterocycle formed from R¹ and R² may carry one, two, three or four identical or different groups R^{a} and/or two substituents attached to adjacent ring atoms may represent (C₁-C₆)-alkylene, oxy-(C₂-C₄)-alkylene or oxy-(C₁-C₃)-alkyleneoxy;
wherein R^{a} is as defined below:
R^{a} is halogen, cyano, nitro, hydroxyl, carboxyl, amino, (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₄-C₁₀)-alkadienyl, (C₁-C₈)-alkoxy, (C₁-C₈)-haloalkoxy, (C₂-C₈)-alkenyloxy, (C₂-C₈)-haloalkenyloxy, (C₂-C₈)-alkynyloxy, (C₂-C₈)-haloalkynyloxy, (C₃-C₈)-cycloalkyl, (C₃-C₈)-halocycloalkyl, (C₃-C₈)-cycloalkenyl, (C₃-C₈)-cycloalkoxy, (C₃-C₈)-halocycloalkoxy, (C₃-C₈)-cycloalkenyloxy, (C₅-C₁₀)-bicycloalkyl, (C₁-C₈)-alkylcarbonyl, (C₁-C₈)-alkoxycarbonyl, (C₁-C₈)-alkylcarbonyloxy, (C₁-C₈)-alkylamino, di-(C₁-C₈)-alkylamino, (C₁-C₈)-alkylaminocarbonyl, di-(C₁-C₈)-alkylaminocarbonyl, (C₁-C₈)-alkylaminothiocarbonyl, di-(C₁-C₈)-alkylaminothiocarbonyl, oxy-(C₁-C₆)-alkyleneoxy, (C₁-C₈)-alkylthio, (C₁-C₈)-alkylsulfinyl, (C₁-C₈)-alkylsulfonyl, (C₁-C₈)-alkylsulfoxyl, phenyl, naphthyl, a five- to ten-membered saturated, partially unsaturated or aromatic heterocycle which contains one, two, three or four heteroatoms from the group consisting of O, N and S;
wherein the aliphatic, alicyclic or aromatic groups in R^{a} may carry one, two or three identical or different groups R^{b}:
R^{b} is halogen, cyano, nitro, hydroxyl, carboxyl, mercapto, amino, formyl, aminocarbonyl, aminothiocarbonyl, (C₁-C₈)-alkyl, (C₁-C₈)-haloalkyl, (C₂-C₈)-alkenyl, (C₂-C₈)-haloalkenyl, (C₄-C₁₀)-alkadienyl, (C₂-C₈)-alkynyl, (C₂-C₈)-haloalkynyl, (C₁-C₈)-alkoxy, (C₁-C₈)-haloalkoxy, (C₂-C₈)-alkenyloxy, (C₂-C₈)-alkynyloxy, (C₃-C₈)-cycloalkyl, (C₃-C₈)-cycloalkenyl, (C₃-C₈)-cycloalkoxy, (C₃-C₈)-cycloalkenyloxy, (C₅-C₁₀)-bicycloalkyl, amino, (C₁-C₈)-alkylamino, di-(C₁-C₈)-alkylamino, (C₁-C₈)-alkylcarbonyl, (C₁-C₈)-alkoxycarbonyl, (C₁-C₈)-alkylcarbonyloxy, (C₁-C₈)-alkylaminocarbonyl, di-(C₁-C₈)-alkylaminocarbonyl, (C₁-C₈)-alkylaminothiocarbonyl, di-(C₁-C₈)-alkylaminothiocarbonyl, (C₁-C₈)-alkylthio, (C₁-C₈)-alkylsulfinyl, (C₁-C₈)-alkylsulfonyl, (C₁-C₈)-alkylsulfoxyl, (C₆-C₁₄)-aryl, (C₆-C₁₄)-haloaryl, (C₆-C₁₄)—aryloxy, (C₆-C₁₄)-arylthio, (C₆-C₁₄)-aryl-(C₁-C₆)-alkoxy, (C₆-C₁₄)-aryl-(C₁-C₆)-alkyl, a five- to ten-membered saturated, partially unsaturated or aromatic heterocycle which contains one, two, three or four heteroatoms from the group consisting of O, N and S, heterocyclyloxy, heteroaryloxy, heteroarylthio, wherein the cyclic systems may be partially or fully halogenated and/or substituted by (C₁-C₈)-alkyl and/or (C₁-C₈)-haloalkyl groups;
X is (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl; cyano-(C₁-C₄)-alkyl, or (C₁-C₄)-alkoxy-(C₁-C₄)-alkyl;
Y is hydrogen, halogen, cyano, (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₁-C₈)-alkoxy, (C₁-C₈)-haloalkoxy, (C₃-C₈)-cycloalkyl, (C₃-C₈)-halocycloalkyl, (C₁-C₈)-alkylthio, (C₁-C₈)-alkylsulfinyl or (C₁-C₉)-alkylsulfonyl;
or an agriculturally acceptable salt thereof.

22. The compound according to claim 21, wherein at least one of R¹ and R² is different from hydrogen.

23. The compound according to claim 21, wherein R¹ and R² together with the nitrogen atom to which they are attached form a five- or six-membered heterocyclyl or heteroaryl which is attached via N and which may contain one, two or three further heteroatoms from the group consisting of O, N and S as ring members, wherein the heterocyclyl or heteroaryl may be unsubstituted or substituted by one or two identical or different substituents R^{a}, wherein R^{a} is selected from the group consisting of halogen, (C₁-C₆)-alkyl and (C₁-C₆)-haloalkyl.

24. The compound according to claim 21, wherein R¹ and R² are hydrogen.

25. The compound according to claim 21, wherein X is (C₁-C₄)-alkyl or (C₁-C₄)-haloalkyl.

26. The compound according to claim 21, wherein Het contains at least one substituent in the position ortho to the point of attachment to the pyrimidine skeleton.

27. A composition for controlling phytopathogenic fungi, wherein the composition comprises at least one 7-amino-6-heteroaryl-1,2,4-triazolo[1,5-a]pyrimidine compound of formula (I) 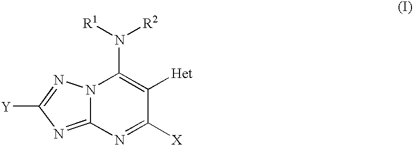
wherein,
Het is a five-membered aromatic heterocycle containing one, two, three or four heteroatoms from the group consisting of oxygen, nitrogen and sulfur, wherein Het is unsubstituted or substituted by one, two, three or four identical or different substituents L:
L is halogen, cyano, nitro, hydroxyl, cyanato (OCN), (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₄-C₁₀)-alkadienyl, (C₄-C₁₀)-haloalkadienyl, (C₁-C₈)-alkoxy, (C₁-C₈)-haloalkoxy, (C₂-C₈)-alkenyloxy, (C₂-C₈)-haloalkenyloxy, (C₂-C₈)-alkynyloxy, (C₂-C₈)-haloalkynyloxy, (C₃-C₈)-cycloalkyl, (C₃-C₈)-halocycloalkyl, (C₃-C₈)-cycloalkenyl, (C₃-C₈)-halocycloalkenyl, (C₃-C₈)-cycloalkoxy, (C₁-C₈)-alkoximino-(C₁-C₈)-alkyl, (C₂-C₈)-alkenyloximino-(C₁-C₈)-alkyl, (C₂-C₈)-alkynyloximino-(C₁-C₈)-alkyl, S(═O)ₙA¹, C(═O)A², C(═S)A², NR⁵R⁶, or NR⁵—(C═O)—R⁶; where
n is 0, 1 or 2;
A¹ is hydrogen, hydroxyl, (C₁-C₈)-alkyl, (C₁-C₈)-haloalkyl, amino, (C₁-C₈)-alkylamino or di-(C₁-C₈)-alkylamino,
A² is one of the groups mentioned under A¹ or (C₂-C₈)-alkenyl, (C₂-C₈)-haloalkenyl, (C₂-C₈)-alkynyl, (C₂-C₈)-haloalkynyl, (C₁-C₈)-alkoxy, (C₁-C₈)-haloalkoxy, (C₂-C₈)-alkenyloxy, (C₂-C₈)-haloalkenyloxy, (C₂-C₈)-alkynyloxy, (C₂-C₈)-haloalkynyloxy, (C₃-C₈)-cycloalkoxy, or (C₃-C₈)-halocycloalkoxy;
R⁵, R⁶ independently of one another are hydrogen, (C₁-C₉)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₃-C₈)-cycloalkyl, (C₃-C₈)-halocycloalkyl, (C₃-C₈)-cycloalkenyl or (C₃-C₈)-halocycloalkenyl,
wherein the aliphatic groups of the radicals of L may carry one to four identical or different groups R^{L}:
R^{L} is halogen, cyano, (C₁-C₈)-alkoxy, (C₂-C₈)-alkenyloxy, (C₂-C₈)-alkynyloxy, (C₃-C₈)-cycloalkyl, (C₃-C₈)-cycloalkenyl, (C₃-C₈)-cycloalkyloxy, (C₃-C₈)-cycloalkenyloxy, (C₁-C₈)-alkoximino-(C₁-C₈)-alkyl, (C₂-C₈)-alkenyloximino-(C₁-C₈)-alkyl, (C₂-C₈)-alkynyloximino-(C₁-C₈)-alkyl, S(═O)ₙA¹, C(═O)A², C(═S)A², NR⁵R⁶, or NR⁵—(C═O)—R⁶;
R¹, R² independently of one another are hydrogen, (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₄-C₁₀)-alka dienyl, (C₄-C₁₀)-haloalkadienyl, (C₁-C₈)-alkoxy, (C₁-C₈)-haloalkoxy, (C₂-C₈)-alkenyloxy, (C₂-C₈)-haloalkenyloxy, (C₂-C₈)-alkynyloxy, (C₂-C₈)-haloalkynyloxy, (C₃-C₈)-cycloalkyl, (C₃-C₈)-halocycloalkyl, (C₃-C₈)-cycloalkenyl, (C₃-C₈)-halocycloalkenyl, (C₃-C₈)-cycloalkoxy, (C₃-C₈)-halocycloalkoxy, (C₅-C₁₀)-bicycloalkyl, amino, (C₁-C₈)-alkylamino, di-(C₁-C₈)-alkylamino, phenyl, naphthyl or a five- or six-membered saturated, partially unsaturated or aromatic heterocycle which contains one, two, three or four heteroatoms from the group consisting of O, N and S;
R¹ and R² together with the nitrogen atom to which they are attached may also form a five- or six-membered saturated, partially unsaturated or aromatic heterocycle which may contain one, two or three further heteroatoms from the group consisting of O, N and S as ring members, wherein
R¹ and/or R² or a heterocycle formed from R¹ and R² may carry one, two, three or four identical or different groups R^{a} and/or two substituents attached to adjacent ring atoms may represent (C₁-C₆)-alkylene, oxy-(C₂-C₄)-alkylene or oxy-(C₁-C₃)-alkyleneoxy; wherein R^{a} is as defined below:
R^{a} is halogen, cyano, nitro, hydroxyl, carboxyl, amino, (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₄-C₁₀)-alkadienyl, (C₁-C₈)-alkoxy, (C₁-C₈)-haloalkoxy, (C₂-C₈)-alkenyloxy, (C₂-C₈)-haloalkenyloxy, (C₂-C₈)-alkynyloxy, (C₂-C₈)-haloalkynyloxy, (C₃-C₈)-cycloalkyl, (C₃-C₈)-halocycloalkyl, (C₃-C₈)-cycloalkenyl, (C₃-C₈)-cycloalkoxy, (C₃-C₈)-halocycloalkoxy, (C₃-C₈)-cycloalkenyloxy, (C₅-C₁₀)-bicycloalkyl, (C₁-C₈)-alkylcarbonyl, (C₁-C₈)-alkoxycarbonyl, (C₁-C₈)-alkylcarbonyloxy, (C₁-C₈)-alkylamino, di-(C₁-C₈)-alkylamino, (C₁-C₈)-alkylaminocarbonyl, di-(C₁-C₈)-alkylaminocarbonyl, (C₁-C₈)-alkylaminothiocarbonyl, di-(C₁-C₈)-alkylaminothiocarbonyl, oxy-(C₁-C₆)-alkyleneoxy, (C₁-C₈)-alkylthio, (C₁-C₈)-alkylsulfinyl, (C₁-C₈)-alkylsulfonyl, (C₁-C₈)-alkylsulfoxyl, phenyl, naphthyl, a five- to ten-membered saturated, partially unsaturated or aromatic heterocycle which contains one, two, three or four heteroatoms from the group consisting of O, N and S;
wherein the aliphatic, alicyclic or aromatic groups in R may carry one, two or three identical or different groups R^{b}:
R^{b} is halogen, cyano, nitro, hydroxyl, carboxyl, mercapto, amino, formyl, aminocarbonyl, aminothiocarbonyl, (C₁-C₈)-alkyl, (C₁-C₈)-haloalkyl, (C₂-C₈)-alkenyl, (C₂-C₈)-haloalkenyl, (C₄-C₁₀)-alkadienyl, (C₂-C₈)-alkynyl, (C₂-C₈)-haloalkynyl, (C₁-C₈)-alkoxy, (C₁-C₈)-haloalkoxy, (C₂-C₈)-alkenyloxy, (C₂-C₈)-alkynyloxy, (C₃-C₈)-cycloalkyl, (C₃-C₈)-cycloalkenyl, (C₃-C₈)-cycloalkoxy, (C₃-C₈)-cycloalkenyloxy, (C₅-C₁₀)-bicycloalkyl, amino, (C₁-C₈)-alkylamino, di-(C₁-C₈)-alkylamino, (C₁-C₈)-alkylcarbonyl, (C₁-C₈)-alkoxycarbonyl, (C₁-C₈)-alkylcarbonyloxy, (C₁-C₈)-alkylaminocarbonyl, di-(C₁-C₈)-alkylaminocarbonyl, (C₁-C₈)-alkylaminothiocarbonyl, di-(C₁-C₈)-alkylaminothiocarbonyl, (C₁-C₈)-alkylthio, (C₁-C₈)-alkylsulfinyl, (C₁-C₈)-alkylsulfonyl, (C₁-C₈)-alkylsulfoxyl, (C₆-C₁₄)-aryl, (C₆-C₁₄)-haloaryl, (C₆-C₁₄)-aryloxy, (C₆-C₁₄)-arylthio, (C₆-C₄)-aryl-(C₁-C₆)-alkoxy, (C₆-C₁₄)-aryl-(C₁-C₆)-alkyl, a five- to ten-membered saturated, partially unsaturated or aromatic heterocycle which contains one, two, three or four heteroatoms from the group consisting of O, N and S, heterocyclyloxy, heteroaryloxy, heteroarylthio, wherein the cyclic systems may be partially or fully halogenated and/or substituted by (C₁-C₈)-alkyl and/or (C₁-C₈)-haloalkyl groups;
X is (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl; cyano-(C₁-C₄)-alkyl, or (C₁-C₄)-alkoxy-(C₁-C₄)-alkyl;
Y is hydrogen, halogen, cyano, (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₁-C₈)-alkoxy, (C₁-C₈)-haloalkoxy, (C₃-C₈)-cycloalkyl, (C₃-C₈)-halocycloalkyl, (C₁-C₈)-alkylthio, (C₁-C₈)-alkylsulfinyl or (C₁-C₈)-alkylsulfonyl;
or an agriculturally acceptable salt thereof and at least one solid or liquid carrier.

28. A composition comprising at least one compound of formula (I) 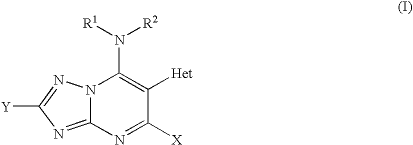
where,
Het is a five-membered aromatic heterocycle containing one, two, three or four heteroatoms from the group consisting of oxygen, nitrogen and sulfur, wherein Het is unsubstituted or substituted by one, two, three or four identical or different substituents L:
L is halogen, cyano, nitro, hydroxyl, cyanato (OCN), (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₄-C₁₀)-alkadienyl, (C₄-C₁₀)-haloalkadienyl, (C₁-C₈)-alkoxy, (C₁-C₈)-haloalkoxy, (C₂-C₈)-alkenyloxy, (C₂-C₈)-haloalkenyloxy, (C₂-C₈)-alkynyloxy, (C₂-C₈)-haloalkynyloxy, (C₃-C₈)-cycloalkyl, (C₃-C₈)-halocycloalkyl, (C₃-C₈)-cycloalkenyl, (C₃-C₈)-halocycloalkenyl, (C₃-C₈)-cycloalkoxy, (C₁-C₈)-alkoximino-(C₁-C₈)-alkyl, (C₂-C₈)-alkenyloximino-(C₁-C₈)-alkyl, (C₂-C₈)-alkynyloximino-(C₁-C₈)-alkyl, S(═O)ₙA¹, C(═O)A², C(═S)A², NR⁵R⁶, or NR⁵—(C═O)—R⁶; wherein
n is 0, 1 or 2;
A¹ is hydrogen, hydroxyl, (C₁-C₈)-alkyl, (C₁-C₈)-haloalkyl, amino, (C₁-C₈)-alkylamino or di-(C₁-C₈)-alkylamino,
A² is one of the groups mentioned under A or (C₂-C₈)-alkenyl, (C₂-C₈)-haloalkenyl, (C₂-C₈)-alkynyl, (C₂-C₈)-haloalkynyl, (C₁-C₈)-alkoxy, (C₁-C₈)-haloalkoxy, (C₂-C₈)-alkenyloxy, (C₂-C₈)-haloalkenyloxy, (C₂-C₈)-alkynyloxy, (C₂-C₈)-haloalkynyloxy, (C₃-C₈)-cycloalkoxy, or (C₃-C₈)-halocycloalkoxy;
R⁵, R¹ independently of one another are hydrogen, (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₃-C₈)-cycloalkyl, (C₃-C₈)-halocycloalkyl, (C₃-C₈)-cycloalkenyl or (C₃-C₈)-halocycloalkenyl,
wherein the aliphatic groups of the radicals of L may carry one to four identical or different groups R^{L}:
R^{L} is halogen, cyano, (C₁-C₈)-alkoxy, (C₂-C₈)-alkenyloxy, (C₂-C₈)-alkynyloxy, (C₃-C₈)-cycloalkyl, (C₃-C₈)-cycloalkenyl, (C₃-C₈)-cycloalkyloxy, (C₃-C₈)-cycloalkenyloxy, (C₁-C₈)-alkoximino-(C₁-C₈)-alkyl, (C₂-C₈)-alkenyloximino-(C₁-C₈)-alkyl, (C₂-C₈)-alkynyloximino-(C₁-C₈)-alkyl, S(═O)ₙA¹, C(═O)A², C(═S)A , NR⁵R⁶, or NR⁵—(C═O)—R⁶;
R¹, R² independently of one another are hydrogen, (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₄-C₁₀)-alka dienyl, (C₄-C₁₀)-haloalkadienyl, (C₁-C₈)-alkoxy, (C₁-C₈)-haloalkoxy, (C₂-C₈)-alkenyloxy, (C₂-C₈)-haloalkenyloxy, (C₂-C₈)-alkynyloxy, (C₂-C₈)-haloalkynyloxy, (C₃-C₈)-cycloalkyl, (C₃-C₈)-halocycloalkyl, (C₃-C₈)-cycloalkenyl, (C₃-C₈)—halocycloalkenyl, (C₃-C₈)-cycloalkoxy, (C₃-C₈)-halocycloalkoxy, (C₅-C₁₀)-bicycloalkyl, amino, (C₁-C₈)-alkylamino, di-(C₁-C₈)-alkylamino, phenyl, naphthyl or a five- or six-membered saturated, partially unsaturated or aromatic heterocycle which contains one, two, three or four heteroatoms from the group consisting of O, N and S;
R¹ and R² together with the nitrogen atom to which they are attached may also form a five- or six-membered saturated, partially unsaturated or aromatic heterocycle which may contain one, two or three further heteroatoms from the group consisting of O, N and S as ring members, wherein
R¹ and/or R² or a heterocycle formed from R¹ and R² may carry one, two, three or four identical or different groups R^{a} and/or two substituents attached to adjacent ring atoms may represent (C₁-C₆)-alkylene, oxy-(C₂-C₄)-alkylene or oxy-(C₁-C₃)-alkyleneoxy;
wherein R^{a} is as defined below:
R^{a} is halogen, cyano, nitro, hydroxyl, carboxyl, amino, (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₄-C₁₀)-alkadienyl, (C₁-C₈)-alkoxy, (C₁-C₈)-haloalkoxy, (C₂-C₈)-alkenyloxy, (C₂-C₈)-haloalkenyloxy, (C₂-C₈)-alkynyloxy, (C₂-C₈)-haloalkynyloxy, (C₃-C₈)-cycloalkyl, (C₃-C₈)-halocycloalkyl, (C₃-C₈)-cycloalkenyl, (C₃-C₈)-cycloalkoxy, (C₃-C₈)-halocycloalkoxy, (C₃-C₈)-cycloalkenyloxy, (C₅-C₁₀)-bicycloalkyl, (C₁-C₈)-alkylcarbonyl, (C₁-C₈)-alkoxycarbonyl, (C₁-C₈)-alkylcarbonyloxy, (C₁-C₈)-alkylamino, di-(C₁-C₈)-alkylamino, (C₁-C₈)-alkylaminocarbonyl, di-(C₁-C₈)-alkylaminocarbonyl, (C₁-C₈)-alkylaminothiocarbonyl, di-(C₁-C₈)-alkylaminothiocarbonyl, oxy-(C₁-C₆)-alkyleneoxy, (C₁-C₈)-alkylthio, (C₁-C₈)-alkylsulfinyl, (C₁-C₈)-alkylsulfonyl, (C₁-C₈)-alkylsulfoxyl, phenyl, naphthyl, a five- to ten-membered saturated, partially unsaturated or aromatic heterocycle which contains one, two, three or four heteroatoms from the group consisting of O, N and S;
wherein the aliphatic, alicyclic or aromatic groups in R^{a} may carry one, two or three identical or different groups R^{b}: R^{b} is halogen, cyano, nitro, hydroxyl, carboxyl, mercapto, amino, formyl, aminocarbonyl, aminothiocarbonyl, (C₁-C₈)-alkyl, (C₁-C₈)-haloalkyl, (C₂-C₈)-alkenyl, (C₂-C₈)-haloalkenyl, (C₄-C₁₀)-alkadienyl, (C₂-C₈)-alkynyl, (C₂-C₈)-haloalkynyl, (C₁-C₈)-alkoxy, (C₁-C₈)-haloalkoxy, (C₂-C₈)-alkenyloxy, (C₂-C₈)-alkynyloxy, (C₃-C₈)-cycloalkyl, (C₃-C₈)-cycloalkenyl, (C₃-C₈)-cycloalkoxy, (C₃-C₈)-cycloalkenyloxy, (C₅-C₁₀)-bicycloalkyl, amino, (C₁-C₈)-alkylamino, di-(C₁-C₈)-alkylamino, (C₁-C₈)-alkylcarbonyl, (C₁-C₈)-alkoxycarbonyl, (C₁-C₈)-alkylcarbonyloxy, (C₁-C₈)-alkylaminocarbonyl, di-(C₁-C₈)-alkylaminocarbonyl, (C₁-C₈)-alkylaminothiocarbonyl, di-(C₁-C₈)-alkylaminothiocarbonyl, (C₁-C₈)-alkylthio, (C₁-C₈)-alkylsulfinyl, (C₁-C₈)-alkylsulfonyl, (C₁-C₈)-alkylsulfoxyl, (C₆-C₁₄)-aryl, (C₆-C₁₄)-haloaryl, (C₆-C₁₄)-aryloxy, (C₆-C₁₄)-arylthio, (C₆-C₁₄)-aryl-(C₁-C₆)-alkoxy, (C₆-C₁₄)-aryl-(C₁-C₆)-alkyl, a five- to ten-membered saturated, partially unsaturated or aromatic heterocycle which contains one, two, three or four heteroatoms from the group consisting of O, N and S, heterocyclyloxy, heteroaryloxy, heteroarylthio, wherein the cyclic systems may be partially or fully halogenated and/or substituted by (C₁-C₈)-alkyl and/or (C₁-C₈)-haloalkyl groups;
X is (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl; cyano-(C₁-C₄)-alkyl, or (C₁-C₄)-alkoxy-(C₁-C₄)-alkyl;
Y is hydrogen, halogen, cyano, (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₁-C₈)-alkoxy, (C₁-C₈)-haloalkoxy, (C₃-C₈)-cycloalkyl, (C₃-C₈)-halocycloalkyl, (C₁-C₈)-alkylthio, (C₁-C₈)-alkylsulfinyl or (C₁-C₈)-alkylsulfonyl;
and/or an agriculturally acceptable salt thereof and at least one further fungicidally, insecticidally and/or herbicidally active compound.

29. A method for controlling phytopathogenic fungi, comprising treating the fungi or the materials, plants, the soil or seeds to be protected against fungal attack with an effective amount of at least one 7-amino-6-heteroaryl-1,2,4-triazolo[1,5-a]pyrimidine compound of formula (I) 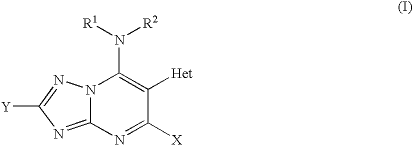
wherein,
Het is a five-membered aromatic heterocycle containing one, two, three or four heteroatoms from the group consisting of oxygen, nitrogen and sulfur, wherein Het is unsubstituted or substituted by one, two, three or four identical or different substituents L:
L is halogen, cyano, nitro, hydroxyl, cyanato (OCN), (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₄-C₁₀)-alkadienyl, (C₄-C₁₀)-haloalkadienyl, (C₁-C₈)-alkoxy, (C₁-C₈)-haloalkoxy, (C₂-C₈)-alkenyloxy, (C₂-C₈)-haloalkenyloxy, (C₂-C₈)-alkynyloxy, (C₂-C₈)-haloalkynyloxy, (C₃-C₈)-cycloalkyl, (C₃-C₈)-halocycloalkyl, (C₃-C₈)-cycloalkenyl, (C₃-C₈)-halocycloalkenyl, (C₃-C₈)-cycloalkoxy, (C₁-C₈)-alkoximino-(C₁-C₈)-alkyl, (C₂-C₈)-alkenyloximino-(C₁-C₈)-alkyl, (C₂-C₈)-alkynyloximino-(C₁-C₈)-alkyl, S(═O)ₙA¹, C(═O)A², C(═S)A², NR⁵R⁶, or NR⁵—(C═O)—R⁶; where
n is 0, 1 or 2;
A¹ is hydrogen, hydroxyl, (C₁-C₈)-alkyl, (C₁-C₈)-haloalkyl, amino, (C₁-C₈)-alkylamino or di-(C₁-C₈)-alkylamino,
A2 is one of the groups mentioned under A¹ or (C₂-C₈)-alkenyl, (C₂-C₈)-haloalkenyl, (C₂-C₈)-alkynyl, (C₂-C₈)-haloalkynyl, (C₁-C₈)-alkoxy, (C₁-C₈)-haloalkoxy, (C₂-C₈)-alkenyloxy, (C₂-C₈)-haloalkenyloxy, (C₂-C₈)-alkynyloxy, (C₂-C₈)-haloalkynyloxy, (C₃-C₈)-cycloalkoxy, or (C₃-C₈)-halocycloalkoxy; R⁵, R⁶ independently of one another are hydrogen, (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₃-C₈)-cycloalkyl, (C₃-C₈)-halocycloalkyl, (C₃-C₈)-cycloalkenyl or (C₃-C₈)-halocycloalkenyl,
wherein the aliphatic groups of the radicals of L may carry one to four identical or different groups R^{L}:
R^{L} is halogen, cyano, (C₁-C₈)-alkoxy, (C₂-C₈)-alkenyloxy, (C₂-C₈)-alkynyloxy, (C₃-C₈)-cycloalkyl, (C₃-C₈)-cycloalkenyl, (C₃-C₈)-cycloalkyloxy, (C₃-C₈)-cycloalkenyloxy, (C₁-C₈)-alkoximino-(C₁-C₈)-alkyl, (C₂-C₈)-alkenyloximino-(C₁-C₈)-alkyl, (C₂-C₈)-alkynyloximino-(C₁-C₈)-alkyl, S(═O)ₙA¹, C(═O)ₙA¹, C(═S)A², NR⁵R⁵, or NR⁵—(C═O)—R⁶;
R¹, R² independently of one another are hydrogen, (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₄-C₁₀)-alka dienyl, (C₄-C₁₀)-haloalkadienyl, (C₁-C₈)-alkoxy, (C₁-C₈)-haloalkoxy, (C₂-C₈)-alkenyloxy, (C₂-C₈)-haloalkenyloxy, (C₂-C₈)-alkynyloxy, (C₂-C₈)-haloalkynyloxy, (C₃-C₈)-cycloalkyl, (C₃-C₈)-halocycloalkyl, (C₃-C₈)-cycloalkenyl, (C₃-C₈)-halocycloalkenyl, (C₃-C₈)-cycloalkoxy, (C₃-C₈)-halocycloalkoxy, (C₅-C₁₀)-bicycloalkyl, amino, (C₁-C₈)-alkylamino, di-(C₁-C₈)-alkylamino, phenyl, naphthyl or a five- or six-membered saturated, partially unsaturated or aromatic heterocycle which contains one, two, three or four heteroatoms from the group consisting of O, N and S;
R¹ and R² together with the nitrogen atom to which they are attached may also form a five- or six-membered saturated, partially unsaturated or aromatic heterocycle which may contain one, two or three further heteroatoms from the group consisting of O, N and S as ring members, wherein R¹ and/or R² or a heterocycle formed from R¹ and R² may carry one, two, three or four identical or different groups R^{a} and/or two substituents attached to adjacent ring atoms may represent (C₁-C₆)-alkylene, oxy-(C₂-C₄)-alkylene or oxy-(C₁-C₃)-alkyleneoxy; wherein R^{a} is as defined below:
R^{a} is halogen, cyano, nitro, hydroxyl, carboxyl, amino, (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₄-C₁₀)—alkadienyl, (C₁-C₈)-alkoxy, (C₁-C₈)-haloalkoxy, (C₂-C₈)-alkenyloxy, (C₂-C₈)-haloalkenyloxy, (C₂-C₈)-alkynyloxy, (C₂-C₈)-haloalkynyloxy, (C₃-C₈)-cycloalkyl, (C₃-C₈)-halocycloalkyl, (C₃-C₈)-cycloalkenyl, (C₃-C₈)-cycloalkoxy, (C₃-C₈)-halocycloalkoxy, (C₃-C₈)-cycloalkenyloxy, (C₅-C₁₀)-bicycloalkyl, (C₁-C₈)-alkylcarbonyl, (C₁-C₈)-alkoxycarbonyl, (C₁-C₈)-alkylcarbonyloxy, (C₁-C₈)-alkylamino, di-(C₁-C₈)-alkylamino, (C₁-C₈)-alkylaminocarbonyl, di-(C₁-C₈)-alkylaminocarbonyl, (C₁-C₈)-alkylaminothiocarbonyl, di-(C₁-C₈)-alkylaminothiocarbonyl, oxy-(C₁-C₆)-alkyleneoxy, (C₁-C₈)-alkylthio, (C₁-C₈)-alkylsulfinyl, (C₁-C₈)-alkylsulfonyl, (C₁-C₈)-alkylsulfoxyl, phenyl, naphthyl, a five- to ten-membered saturated, partially unsaturated or aromatic heterocycle which contains one, two, three or four heteroatoms from the group consisting of O, N and S;
wherein the aliphatic, alicyclic or aromatic groups in R may carry one, two or three identical or different groups R^{b}:
R^{b} is halogen, cyano, nitro, hydroxyl, carboxyl, mercapto, amino, formyl, aminocarbonyl, aminothiocarbonyl, (C₁-C₈)-alkyl, (C₁-C₈)-haloalkyl, (C₂-C₈)-alkenyl, (C₂-C₈)-haloalkenyl, (C₄-C₁₀)-alkadienyl, (C₂-C₈)-alkynyl, (C₂-C₈)-haloalkynyl, (C₁-C₈)-alkoxy, (C₁-C₈)-haloalkoxy, (C₂-C₈)-alkenyloxy, (C₂-C₈)-alkynyloxy, (C₃-C₈)-cycloalkyl, (C₃-C₈)-cycloalkenyl, (C₃-C₈)-cycloalkoxy, (C₃-C₈)-cycloalkenyloxy, (C₅-C₁₀)-bicycloalkyl, amino, (C₁-C₈)-alkylamino, di-(C₁-C₈)-alkylamino, (C₁-C₈)-alkylcarbonyl, (C₁-C₈)-alkoxycarbonyl, (C₁-C₈)-alkylcarbonyloxy, (C₁-C₈)-alkylaminocarbonyl, di-(C₁-C₈)-alkylaminocarbonyl, (C₁-C₈)-alkylaminothiocarbonyl, di-(C₁-C₈)-alkylaminothiocarbonyl, (C₁-C₈)-alkylthio, (C₁-C₈)-alkylsulfinyl, (C₁-C₈)-alkylsulfonyl, (C₁-C₈)-alkylsulfoxyl, (C₆-C₁₄)-aryl, (C₆-C₁₄)-haloaryl, (C₆-C₁₄)-aryloxy, (C₆-C₄)-arylthio, (C₆-C₁₄)-aryl-(C₁-C₆)-alkoxy, (C₆-C₁₄)-aryl-(C₁-C₆)-alkyl, a five- to ten-membered saturated, partially unsaturated or aromatic heterocycle which contains one, two, three or four heteroatoms from the group consisting of O, N and S, heterocyclyloxy, heteroaryloxy, heteroarylthio, wherein the cyclic systems may be partially or fully halogenated and/or substituted by (C₁-C₈)-alkyl and/or (C₁-C₈)-haloalkyl groups;
X is (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl; cyano-(C₁-C₄)-alkyl, or (C₁-C₄)-alkoxy-(C₁-C₄)-alkyl;
Y is hydrogen, halogen, cyano, (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₁-C₈)-alkoxy, (C₁-C₈)-haloalkoxy, (C₃-C₈)-cycloalkyl, (C₃-C₈)-halocycloalkyl, (C₁-C₈)-alkylthio, (C₁-C₈)-alkylsulfinyl or (C₁-C₈)-alkylsulfonyl;
or an agriculturally acceptable salt thereof.

30. Seed, comprising a 7-amino-6-heteroaryl-1,2,4-triazolo[1,5-a]pyrimidine compound of formula (I) 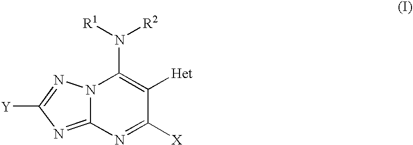
wherein,
Het is a five-membered aromatic heterocycle containing one, two, three or four heteroatoms from the group consisting of oxygen, nitrogen and sulfur, wherein Het is unsubstituted or substituted by one, two, three or four identical or different substituents L:
L is halogen, cyano, nitro, hydroxyl, cyanato (OCN), (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₄-C₁₀)-alkadienyl, (C₄-C₁₀)-haloalkadienyl, (C₁-C₈)-alkoxy, (C₁-C₈)-haloalkoxy, (C₂-C₈)-alkenyloxy, (C₂-C₈)-haloalkenyloxy, (C₂-C₈)-alkynyloxy, (C₂-C₈)-haloalkynyloxy, (C₃-C₈)-cycloalkyl, (C₃-C₈)-halocycloalkyl, (C₃-C₈)-cycloalkenyl, (C₃-C₈)-halocycloalkenyl, (C₃-C₈)-cycloalkoxy, (C₁-C₈)-alkoximino-(C₁-C₈)-alkyl, (C₂-C₈)-alkenyloximino-(C₁-C₈)-alkyl, (C₂-C₈)-alkynyloximino-(C₁-C₈)-alkyl, S(═O)ₙA¹, C(═O)A², C(═S)A², NR⁵R⁶, or NR⁵—(C═O)—R⁶; where
n is 0, 1 or 2;
A¹ is hydrogen, hydroxyl, (C₁-C₈)-alkyl, (C₁-C₈)-haloalkyl, amino, (C₁-C₈)-alkylamino or di-(C₁-C₈)-alkylamino, A2 is one of the groups mentioned under A¹ or (C₂-C₈)-alkenyl, (C₂-C₈)-haloalkenyl, (C₂-C₈)-alkynyl, (C₂-C₈)-haloalkynyl, (C₁-C₈)-alkoxy, (C₁-C₈)-haloalkoxy, (C₂-C₈)-alkenyloxy, (C₂-C₈)-haloalkenyloxy, (C₂-C₈)-alkynyloxy, (C₂-C₈)-haloalkynyloxy, (C₃-C₈)-cycloalkoxy, or (C₃-C₈)-halocycloalkoxy;
R⁵, R⁶ independently of one another are hydrogen, (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₃-C₈)-cycloalkyl, (C₃-C₈)-halocycloalkyl, (C₃-C₈)-cycloalkenyl or (C₃-C₈)-halocycloalkenyl,
wherein the aliphatic groups of the radicals of L may carry one to four identical or different groups R^{L};
R^{L} is halogen, cyano, (C₁-C₈)-alkoxy, (C₂-C₈)-alkenyloxy, (C₂-C₈)-alkynyloxy, (C₃-C₈)-cycloalkyl, (C₃-C₈)-cycloalkenyl, (C₃-C₈)-cycloalkyloxy, (C₃-C₈)-cycloalkenyloxy, (C₁-C₈)-alkoximino-(C₁-C₈)-alkyl, (C₂-C₈)-alkenyloximino-(C₁-C₈)-alkyl, (C₂-C₈)-alkynyloximino-(C₁-C₈)-alkyl, S(═O)ₙA¹, C(═O)A², C(═S)A², NR⁵R⁶, or NR⁵—(C═O)—R⁶.
R¹, R² independently of one another are hydrogen, (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₄-C₁₀)-alka dienyl, (C₄-C₁₀)-haloalkadienyl, (C₁-C₈)-alkoxy, (C₁-C₈)-haloalkoxy, (C₂-C₈)-alkenyloxy, (C₂-C₈)-haloalkenyloxy, (C₂-C₈)-alkynyloxy, (C₂-C₈)-haloalkynyloxy, (C₃-C₈)-cycloalkyl, (C₃-C₈)-halocycloalkyl, (C₃-C₈)-cycloalkenyl, (C₃-C₈)-halocycloalkenyl, (C₃-C₈)-cycloalkoxy, (C₃-C₈)-halocycloalkoxy, (C₅-C₁₀)-bicycloalkyl, amino, (C₁-C₈)-alkylamino, di-(C₁-C₈)-alkylamino, phenyl, naphthyl or a five- or six-membered saturated, partially unsaturated or aromatic heterocycle which contains one, two, three or four heteroatoms from the group consisting of O, N and S;
R¹ and R² together with the nitrogen atom to which they are attached may also form a five- or six-membered saturated, partially unsaturated or aromatic heterocycle which may contain one, two or three further heteroatoms from the group consisting of O, N and S as ring members, wherein R¹ and/or R² or a heterocycle formed from R¹ and R² may carry one, two, three or four identical or different groups R^{a} and/or two substituents attached to adjacent ring atoms may represent (C₁-C₆)-alkylene, oxy-(C₂-C₄)-alkylene or oxy-(C₁-C₃)-alkyleneoxy; wherein R^{a} is as defined below:
R^{a} is halogen, cyano, nitro, hydroxyl, carboxyl, amino, (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₄-C₁₀)-alkadienyl, (C₁-C₈)-alkoxy, (C₁-C₈)-haloalkoxy, (C₂-C₈)-alkenyloxy, (C₂-C₈)-haloalkenyloxy, (C₂-C₈)-alkynyloxy, (C₂-C₈)-haloalkynyloxy, (C₃-C₈)-cycloalkyl, (C₃-C₈)-halocycloalkyl, (C₃-C₈)-cycloalkenyl, (C₃-C₈)-cycloalkoxy, (C₃-C₈)-halocycloalkoxy, (C₃-C₈)-cycloalkenyloxy, (C₅-C₁₀)-bicycloalkyl, (C₁-C₈)-alkylcarbonyl, (C₁-C₈)-alkoxycarbonyl, (C₁-C₈)-alkylcarbonyloxy, (C₁-C₈)-alkylamino, di-(C₁-C₈)-alkylamino, (C₁-C₈)-alkylaminocarbonyl, di-(C₁-C₈)-alkylaminocarbonyl, (C₁-C₈)-alkylaminothiocarbonyl, di-(C₁-C₈)-alkylaminothiocarbonyl, oxy-(C₁-C₆)-alkyleneoxy, (C₁-C₈)-alkylthio, (C₁-C₈)-alkylsulfinyl, (C₁-C₈)-alkylsulfonyl, (C₁-C₈)-alkylsulfoxyl, phenyl, naphthyl, a five- to ten-membered saturated, partially unsaturated or aromatic heterocycle which contains one, two, three or four heteroatoms from the group consisting of O, N and S;
wherein the aliphatic, alicyclic or aromatic groups in R^{a} may carry one, two or three identical or different groups R^{b}:
R^{b} is halogen, cyano, nitro, hydroxyl, carboxyl, mercapto, amino, formyl, aminocarbonyl, aminothiocarbonyl, (C₁-C₈)-alkyl, (C₁-C₈)-haloalkyl, (C₂-C₈)-alkenyl, (C₂-C₈)-haloalkenyl, (C₄-C₁₀)-alkadienyl, (C₂-C₈)-alkynyl, (C₂-C₈)-haloalkynyl, (C₁-C₈)-alkoxy, (C₁-C₈)-haloalkoxy, (C₂-C₈)-alkenyloxy, (C₂-C₈)-alkynyloxy, (C₃-C₈)-cycloalkyl, (C₃-C₈)-cycloalkenyl, (C₃-C₈)-cycloalkoxy, (C₃-C₈)-cycloalkenyloxy, (C₅-C₁₀)-bicycloalkyl, amino, (C₁-C₈)-alkylamino, di-(C₁-C₈)-alkylamino, (C₁-C₈)-alkylcarbonyl, (C₁-C₈)-alkoxycarbonyl, (C₁-C₈)-alkylcarbonyloxy, (C₁-C₈)-alkylaminocarbonyl, di-(C₁-C₈)-alkylaminocarbonyl, (C₁-C₈)-alkylaminothiocarbonyl, di-(C₁-C₈)-alkylaminothiocarbonyl, (C₁-C₈)-alkylthio, (C₁-C₈)-alkylsulfinyl, (C₁-C₈)-alkylsulfonyl, (C₁-C₈)-alkylsulfoxyl, (C₆-C₁₄)-aryl, (C₆-C₁₄)-haloaryl, (C₆-C₁₄)-aryloxy, (C₆-C₁₄)-arylthio, (C₆-C₁₄)-aryl-(C₁-C₆)-alkoxy, (C₆-C₁₄)-aryl-(C₁-C₆)-alkyl, a five- to ten-membered saturated, partially unsaturated or aromatic heterocycle which contains one, two, three or four heteroatoms from the group consisting of O, N and S, heterocyclyloxy, heteroaryloxy, heteroarylthio, wherein the cyclic systems may be partially or fully halogenated and/or substituted by (C₁-C₈)-alkyl and/or (C₁-C₈)-haloalkyl groups;
X is (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl; cyano-(C₁-C₄)-alkyl, or (C₁-C₄)-alkoxy-(C₁-C₄)-alkyl;
Y is hydrogen, halogen, cyano, (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₁-C₈)-alkoxy, (C₁-C₈)-haloalkoxy, (C₃-C₈)-cycloalkyl, (C₃-C₈)-halocycloalkyl, (C₁-C₈)-alkylthio, (C₁-C₈)-alkylsulfinyl or (C₁-C₈)-alkylsulfonyl;
or an agriculturally acceptable salt thereof in an amount of from 1 to 1000 g per 100 kg.

31. A process for preparing a 7-amino-6-heteroaryl-1,2,4-triazolo[1,5-a]pyrimidine compound of the formula (I) 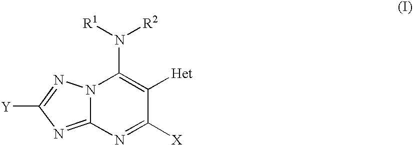
wherein,
Het is a five-membered aromatic heterocycle containing one, two, three or four heteroatoms from the group consisting of oxygen, nitrogen and sulfur, wherein Het is unsubstituted or substituted by one, two, three or four identical or different substituents L:
L is halogen, cyano, nitro, hydroxyl, cyanato (OCN), (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₄-C₁₀)-alkadienyl, (C₄-C₁₀)-haloalkadienyl, (C₁-C₉)-alkoxy, (C₁-C₈)-haloalkoxy, (C₂-C₈)-alkenyloxy, (C₂-C₈)-haloalkenyloxy, (C₂-C₈)-alkynyloxy, (C₂-C₈)-haloalkynyloxy, (C₃-C₈)-cycloalkyl, (C₃-C₈)-halocycloalkyl, (C₃-C₈)-cycloalkenyl, (C₃-C₈)-halocycloalkenyl, (C₃-C₈)-cycloalkoxy, (C₁-C₈)-alkoximino-(C₁-C₈)-alkyl, (C₂-C₈)-alkenyloximino-(C₁-C₈)-alkyl, (C₂-C₈)-alkynyloximino-(C₁-C₈)-alkyl, S(═O)ₙA¹, C(═O)A², C(═S)A , NR⁵R⁶, or NR⁵—(C═O)—R⁶; wherein
n is 0, 1 or 2;
A¹ is hydrogen, hydroxyl, (C₁-C₈)-alkyl, (C₁-C₈)-haloalkyl, amino, (C₁-C₈)-alkylamino or di-(C₁-C₈)-alkylamino, A2 is one of the groups mentioned under A¹ or (C₂-C₈)-alkenyl, (C₂-C₈)-haloalkenyl, (C₂-C₈)-alkynyl, (C₂-C₈)-haloalkynyl, (C₁-C₈)-alkoxy, (C₁-C₈)-haloalkoxy, (C₂-C₈)-alkenyloxy, (C₂-C₈)-haloalkenyloxy, (C₂-C₈)-alkynyloxy, (C₂-C₈)-haloalkynyloxy, (C₃-C₈)-cycloalkoxy, or (C₃-C₈)-halocycloalkoxy;
R⁵, R⁶ independently of one another are hydrogen, (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₃-C₈)-cycloalkyl, (C₃-C₈)-halocycloalkyl, (C₃-C₈)-cycloalkenyl or (C₃-C₈)-halocycloalkenyl,
wherein the aliphatic groups of the radicals of L may carry one to four identical or different groups R^{L}:
R^{L} is halogen, cyano, (C₁-C₈)-alkoxy, (C₂-C₈)-alkenyloxy, (C₂-C₈)-alkynyloxy, (C₃-C₈)-cycloalkyl, (C₃-C₈)-cycloalkenyl, (C₃-C₈)-cycloalkyloxy, (C₃-C₈)-cycloalkenyloxy, (C₁-C₈)-alkoximino-(C₁-C₈)-alkyl, (C₂-C₈)-alkenyloximino-(C₁-C₈)-alkyl, (C₂-C₈)-alkynyloximino-(C₁-C₈)-alkyl, S(═O)ₙA¹, C(═O)A², C(═S)A², NR¹R⁶, or NR⁵—(C═O)—R⁶;
R¹, R² independently of one another are hydrogen, (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₄-C₁₀)-alka dienyl, (C₄-C₁₀)-haloalkadienyl, (C₁-C₈)-alkoxy, (C₁-C₈)-haloalkoxy, (C₂-C₈)-alkenyloxy, (C₂-C₈)-haloalkenyloxy, (C₂-C₈)-alkynyloxy, (C₂-C₈)-haloalkynyloxy, (C₃-C₈)-cycloalkyl, (C₃-C₈)-halocycloalkyl, (C₃-C₈)-cycloalkenyl, (C₃-C₈)-halocycloalkenyl, (C₃-C₈)-cycloalkoxy, (C₃-C₈)-halocycloalkoxy, (C₅-C₁₀)-bicycloalkyl, amino, (C₁-C₈)-alkylamino, di-(C₁-C₈)-alkylamino, phenyl, naphthyl or a five- or six-membered saturated, partially unsaturated or aromatic heterocycle which contains one, two, three or four heteroatoms from the group consisting of O, N and S;
R¹ and R² together with the nitrogen atom to which they are attached may also form a five- or six-membered saturated, partially unsaturated or aromatic heterocycle which may contain one, two or three further heteroatoms from the group consisting of O, N and S as ring members, wherein R¹ and/or R² or a heterocycle formed from R¹ and R² may carry one, two, three or four identical or different groups R^{a} and/or two substituents attached to adjacent ring atoms may represent (C₁-C₆)-alkylene, oxy-(C₂-C₄)-alkylene or oxy-(C₁-C₃)-alkyleneoxy; wherein R^{a} is as defined below:
R^{a} is halogen, cyano, nitro, hydroxyl, carboxyl, amino, (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₄-C₁₀)-alkadienyl, (C₁-C₈)-alkoxy, (C₁-C₈)-haloalkoxy, (C₂-C₈)-alkenyloxy, (C₂-C₈)-haloalkenyloxy, (C₂-C₈)-alkynyloxy, (C₂-C₈)-haloalkynyloxy, (C₃-C₈)-cycloalkyl, (C₃-C₈)-halocycloalkyl, (C₃-C₈)-cycloalkenyl, (C₃-C₈)-cycloalkoxy, (C₃-C₈)-halocycloalkoxy, (C₃-C₈)-cycloalkenyloxy, (C₅-C₁₀)-bicycloalkyl, (C₁-C₈)-alkylcarbonyl, (C₁-C₈)-alkoxycarbonyl, (C₁-C₈)-alkylcarbonyloxy, (C₁-C₈)-alkylamino, di-(C₁-C₈)-alkylamino, (C₁-C₈)-alkylaminocarbonyl, di-(C₁-C₈)-alkylaminocarbonyl, (C₁-C₈)-alkylaminothiocarbonyl, di-(C₁-C₈)-alkylaminothiocarbonyl, oxy-(C₁-C₆)-alkyleneoxy, (C₁-C₈)-alkylthio, (C₁-C₈)-alkylsulfinyl, (C₁-C₈)-alkylsulfonyl, (C₁-C₈)-alkylsulfoxyl, phenyl, naphthyl, a five- to ten-membered saturated, partially unsaturated or aromatic heterocycle which contains one, two, three or four heteroatoms from the group consisting of O, N and S;
wherein the aliphatic, alicyclic or aromatic groups in R^{a} may carry one, two or three identical or different groups R^{b}:
R^{b} is halogen, cyano, nitro, hydroxyl, carboxyl, mercapto, amino, formyl, aminocarbonyl, aminothiocarbonyl, (C₁-C₈)-alkyl, (C₁-C₈)-haloalkyl, (C₂-C₈)-alkenyl, (C₂-C₈)-haloalkenyl, (C₄-C₁₀)-alkadienyl, (C₂-C₈)-alkynyl, (C₂-C₈)-haloalkynyl, (C₁-C₈)-alkoxy, (C₁-C₈)-haloalkoxy, (C₂-C₈)-alkenyloxy, (C₂-C₈)-alkynyloxy, (C₃-C₈)-cycloalkyl, (C₃-C₈)-cycloalkenyl, (C₃-C₈)-cycloalkoxy, (C₃-C₈)-cycloalkenyloxy, (C₅-C₁₀)-bicycloalkyl, amino, (C₁-C₈)-alkylamino, di-(C₁-C₈)-alkylamino, (C₁-C₈)-alkylcarbonyl, (C₁-C₈)-alkoxycarbonyl, (C₁-C₈)-alkylcarbonyloxy, (C₁-C₈)-alkylaminocarbonyl, di-(C₁-C₈)-alkylaminocarbonyl, (C₁-C₈)-alkylaminothiocarbonyl, di-(C₁-C₈)-alkylaminothiocarbonyl, (C₁-C₈)-alkylthio, (C₁-C₈)-alkylsulfinyl, (C₁-C₈)-alkylsulfonyl, (C₁-C₈)-alkylsulfoxyl, (C₆-C₁₄)-aryl, (C₆-C₁₄)-haloaryl, (C₆-C₁₄)-aryloxy, (C₆-C₁₄)-arylthio, (C₆-C₁₄)-aryl-(C₁-C₆)-alkoxy, (C₆-C₁₄)-aryl-(C₁-C₆)-alkyl, a five- to ten-membered saturated, partially unsaturated or aromatic heterocycle which contains one, two, three or four heteroatoms from the group consisting of O, N and S, heterocyclyloxy, heteroaryloxy, heteroarylthio, wherein the cyclic systems may be partially or fully halogenated and/or substituted by (C₁-C₈)-alkyl and/or (C₁-C₈)-haloalkyl groups;
X is (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl; cyano-(C₁-C₄)-alkyl, or (C₁-C₄)-alkoxy-(C₁-C₄)-alkyl;
Y is hydrogen, halogen, cyano, (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₁-C₈)-alkoxy, (C₁-C₈)-haloalkoxy, (C₃-C₈)-cycloalkyl, (C₃-C₈)-halocycloalkyl, (C₁-C₈)-alkylthio, (C₁-C₈)-alkylsulfinyl or (C₁-C₈)-alkylsulfonyl;
or an agriculturally acceptable salt thereof, said process comprising:
(a) reacting a 7-halo-1,2,4-triazolo[1,5-a]pyrimidine compound of the formula (II) 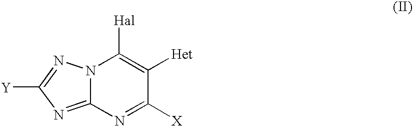
wherein Hal, Het, X, and Y are as described above
with an amine HNR¹R²,
wherein R¹ and R² are as described above,
wherein a compound of formula I is prepared.

32. A process for preparing a 7-halo-1,2,4-triazolo[1,5-a]pyrimidine compound of the formula (II) 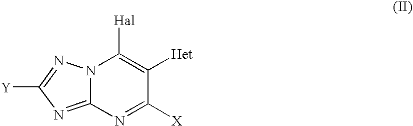
wherein,
Het is a five-membered aromatic heterocycle containing one, two, three or four heteroatoms from the group consisting of oxygen, nitrogen and sulfur, wherein Het is unsubstituted or substituted by one, two, three or four identical or different substituents L:
L is halogen, cyano, nitro, hydroxyl, cyanato (OCN), (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₄-C₁₀)-alkadienyl, (C₄-C₁₀)-haloalkadienyl, (C₁-C₈)-alkoxy, (C₁-C₈)-haloalkoxy, (C₂-C₈)-alkenyloxy, (C₂-C₈)-haloalkenyloxy, (C₂-C₈)-alkynyloxy, (C₂-C₈)-haloalkynyloxy, (C₃-C₈)-cycloalkyl, (C₃-C₈)-halocycloalkyl, (C₃-C₈)-cycloalkenyl, (C₃-C₈)-halocycloalkenyl, (C₃-C₈)-cycloalkoxy, (C₁-C₈)-alkoximino-(C₁-C₈)-alkyl, (C₂-C₈)—alkenyloximino-(C₁-C₈)-alkyl, (C₂-C₈)-alkynyloximino-(C₁-C₈)-alkyl, S(═O)ₙA¹, C(═O)A², C(═S)A², NR⁵R⁶, NR⁵—(C═O)—R⁶; where
n is 0, 1 or 2;
A¹ is hydrogen, hydroxyl, (C₁-C₈)-alkyl, (C₁-C₈)-haloalkyl, amino, (C₁-C₈)-alkylamino or di-(C₁-C₈)-alkylamino, A2 is one of the groups mentioned under A¹ or (C₂-C₈)-alkenyl, (C₂-C₈)-haloalkenyl, (C₂-C₈)-alkynyl, (C₂-C₈)-haloalkynyl, (C₁-C₈)-alkoxy, (C₁-C₈)-haloalkoxy, (C₂-C₈)-alkenyloxy, (C₂-C₈)-haloalkenyloxy, (C₂-C₈)-alkynyloxy, (C₂-C₈)-haloalkynyloxy, (C₃-C₈)-cycloalkoxy, (C₃-C₈)-halocycloalkoxy;
R⁵, R⁶ independently of one another are hydrogen, (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₃-C₈)-cycloalkyl, (C₃-C₈)-halocycloalkyl, (C₃-C₈)-cycloalkenyl or (C₃-C₈)-halocycloalkenyl,
wherein the aliphatic groups of the radicals of L may carry one to four identical or different groups R^{L};
R^{L} is halogen, cyano, (C₁-C₈)-alkoxy, (C₂-C₈)-alkenyloxy, (C₂-C₈)-alkynyloxy, (C₃-C₈)-cycloalkyl, (C₃-C₈)-cycloalkenyl, (C₃-C₈)-cycloalkyloxy, (C₃-C₈)-cycloalkenyloxy, (C₁-C₈)-alkoximino-(C₁-C₈)-alkyl, (C₂-C₈)-alkenyloximino-(C₁-C₈)-alkyl, (C₂-C₈)-alkynyloximino-(C₁-C₈)-alkyl, S(═O)ₙA¹, C(═O)A², C(═S)A², NR⁵R⁶, NR⁵—(C═O)—R⁶;
R¹, R² independently of one another are hydrogen, (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₄-C₁₀)-alka dienyl, (C₄-C₁₀)-haloalkadienyl, (C₁-C₈)-alkoxy, (C₁-C₈)-haloalkoxy, (C₂-C₈)-alkenyloxy, (C₂-C₈)-haloalkenyloxy, (C₂-C₈)-alkynyloxy, (C₂-C₈)-haloalkynyloxy, (C₃-C₈)-cycloalkyl, (C₃-C₈)-halocycloalkyl, (C₃-C₈)-cycloalkenyl, (C₃-C₈)-halocycloalkenyl, (C₃-C₈)-cycloalkoxy, (C₃-C₈)-halocycloalkoxy, (C₅-C₁₀)-bicycloalkyl, amino, (C₁-C₈)-alkylamino, di-(C₁-C₈)-alkylamino, phenyl, naphthyl or a five- or six-membered saturated, partially unsaturated or aromatic heterocycle which contains one, two, three or four heteroatoms from the group consisting of O, N and S;
R¹ and R² together with the nitrogen atom to which they are attached may also form a five- or six-membered saturated, partially unsaturated or aromatic heterocycle which may contain one, two or three further heteroatoms from the group consisting of O, N and S as ring members, where
R¹ and/or R² or a heterocycle formed from R¹ and R² may carry one, two, three or four identical or different groups R^{a} and/or two substituents attached to adjacent ring atoms may represent (C₁-C₆)-alkylene, oxy-(C₂-C₄)-alkylene or oxy-(C₁-C₃)-alkyleneoxy; wherein R^{a} is as defined below:
R^{a} is halogen, cyano, nitro, hydroxyl, carboxyl, amino, (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₄-C₁₀)-alkadienyl, (C₁-C₈)-alkoxy, (C₁-C₈)-haloalkoxy, (C₂-C₈)-alkenyloxy, (C₂-C₈)-haloalkenyloxy, (C₂-C₈)-alkynyloxy, (C₂-C₈)-haloalkynyloxy, (C₃-C₈)-cycloalkyl, (C₃-C₈)-halocycloalkyl, (C₃-C₈)-cycloalkenyl, (C₃-C₈)-cycloalkoxy, (C₃-C₈)-halocycloalkoxy, (C₃-C₈)-cycloalkenyloxy, (C₅-C₁₀)-bicycloalkyl, (C₁-C₈)-alkylcarbonyl, (C₁-C₈)-alkoxycarbonyl, (C₁-C₈)-alkylcarbonyloxy, (C₁-C₈)-alkylamino, di-(C₁-C₈)-alkylamino, (C₁-C₈)-alkylaminocarbonyl, di-(C₁-C₈)-alkylaminocarbonyl, (C₁-C₈)-alkylaminothiocarbonyl, di-(C₁-C₈)-alkylaminothiocarbonyl, oxy-(C₁-C₆)-alkyleneoxy, (C₁-C₈)-alkylthio, (C₁-C₈)-alkylsulfinyl, (C₁-C₈)-alkylsulfonyl, (C₁-C₈)-alkylsulfoxyl, phenyl, naphthyl, a five- to ten-membered saturated, partially unsaturated or aromatic heterocycle which contains one, two, three or four heteroatoms from the group consisting of O, N and S;
wherein the aliphatic, alicyclic or aromatic groups in R^{a} may carry one, two or three identical or different groups R^{b}:
R^{b} is halogen, cyano, nitro, hydroxyl, carboxyl, mercapto, amino, formyl, aminocarbonyl, aminothiocarbonyl, (C₁-C₈)-alkyl, (C₁-C₈)-haloalkyl, (C₂-C₈)-alkenyl, (C₂-C₈)-haloalkenyl, (C₄-C₁₀)-alkadienyl, (C₂-C₈)-alkynyl, (C₂-C₈)-haloalkynyl, (C₁-C₈)-alkoxy, (C₁-C₈)-haloalkoxy, (C₂-C₈)-alkenyloxy, (C₂-C₈)-alkynyloxy, (C₃-C₈)-cycloalkyl, (C₃-C₈)-cycloalkenyl, (C₃-C₈)-cycloalkoxy, (C₃-C₈)-cycloalkenyloxy, (C₅-C₁₀)-bicycloalkyl, amino, (C₁-C₈)-alkylamino, di-(C₁-C₈)-alkylamino, (C₁-C₈)-alkylcarbonyl, (C₁-C₈)-alkoxycarbonyl, (C₁-C₈)-alkylcarbonyloxy, (C₁-C₈)-alkylaminocarbonyl, di-(C₁-C₈)-alkylaminocarbonyl, (C₁-C₈)-alkylaminothiocarbonyl, di-(C₁-C₈)-alkylaminothiocarbonyl, (C₁-C₈)-alkylthio, (C₁-C₈)-alkylsulfinyl, (C₁-C₈)-alkylsulfonyl, (C₁-C₈)-alkylsulfoxyl, (C₆-C₁₄)-aryl, (C₆-C₁₄)-haloaryl, (C₆-C₁₄)-aryloxy, (C₆-C₁₄)-arylthio, (C₆-C₁₄)-aryl-(C₁-C₆)-alkoxy, (C₆-C₁₄)-aryl-(C₁-C₆)-alkyl, a five- to ten-membered saturated, partially unsaturated or aromatic heterocycle which contains one, two, three or four heteroatoms from the group consisting of O, N and S, heterocyclyloxy, heteroaryloxy, heteroarylthio, wherein the cyclic systems may be partially or fully halogenated and/or substituted by (C₁-C₈)-alkyl and/or (C₁-C₈)-haloalkyl groups;
X is (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl; cyano-(C₁-C₄)-alkyl, (C₁-C₄)-alkoxy-(C₁-C₄)-alkyl;
Y is hydrogen, halogen, cyano, (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₁-C₈)-alkoxy, (C₁-C₈)-haloalkoxy, (C₃-C₈)-cycloalkyl, (C₃-C₈)-halocycloalkyl, (C₁-C₈)-alkylthio, (C₁-C₈)-alkylsulfinyl or (C₁-C₈)-alkylsulfonyl,
said method comprising:
(a1) reacting a compound of the formula (III) 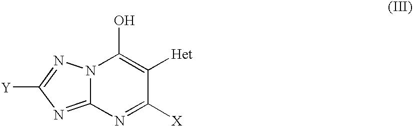
wherein Het, X, and Y are as described above,
with a halogenating agent,
wherein a compound of formula II is prepared.

33. A process for preparing a compound of formula (III) 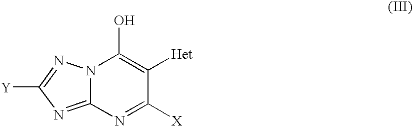
wherein,
Het is a five-membered aromatic heterocycle containing one, two, three or four heteroatoms from the group consisting of oxygen, nitrogen and sulfur, wherein Het is unsubstituted or substituted by one, two, three or four identical or different substituents L:
L is halogen, cyano, nitro, hydroxyl, cyanato (OCN), (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₄-C₁₀)-alkadienyl, (C₄-C₁₀)-haloalkadienyl, (C₁-C₈)-alkoxy, (C₁-C₈)-haloalkoxy, (C₂-C₈)-alkenyloxy, (C₂-C₈)-haloalkenyloxy, (C₂-C₈)-alkynyloxy, (C₂-C₈)-haloalkynyloxy, (C₃-C₈)-cycloalkyl, (C₃-C₈)-halocycloalkyl, (C₃-C₈)-cycloalkenyl, (C₃-C₈)-halocycloalkenyl, (C₃-C₈)-cycloalkoxy, (C₁-C₈)-alkoximino-(C₁-C₈)-alkyl, (C₂-C₈)-alkenyloximino-(C₁-C₈)-alkyl, (C₂-C₈)-alkynyloximino-(C₁-C₈)-alkyl, S(═O)ₙA¹, C(═O)A², C(═S)A², NR⁵R⁶, NR⁵—(C═O)—R⁶; where
n is 0, 1 or 2;
A¹ is hydrogen, hydroxyl, (C₁-C₈)-alkyl, (C₁-C₈)-haloalkyl, amino, (C₁-C₈)-alkylamino or di-(C₁-C₈)-alkylamino, A² is one of the groups mentioned under A¹ or (C₂-C₈)-alkenyl, (C₂-C₈)-haloalkenyl, (C₂-C₈)-alkynyl, (C₂-C₈)-haloalkynyl, (C₁-C₈)-alkoxy, (C₁-C₈)-haloalkoxy, (C₂-C₈)-alkenyloxy, (C₂-C₈)-haloalkenyloxy, (C₂-C₈)-alkynyloxy, (C₂-C₈)-haloalkynyloxy, (C₃-C₈)-cycloalkoxy, (C₃-C₈)-halocycloalkoxy;
R⁵, R⁶ independently of one another are hydrogen, (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₃-C₈)-cycloalkyl, (C₃-C₈)-halocycloalkyl, (C₃-C₈)-cycloalkenyl or (C₃-C₈)-halocycloalkenyl,
wherein the aliphatic groups of the radicals of L may carry one to four identical or
different groups R^{L};
R^{L} is halogen, cyano, (C₁-C₈)-alkoxy, (C₂-C₈)-alkenyloxy, (C₂-C₈)-alkynyloxy, (C₃-C₈)-cycloalkyl, (C₃-C₈)-cycloalkenyl, (C₃-C₈)-cycloalkyloxy, (C₃-C₈)-cycloalkenyloxy, (C₁-C₈)-alkoximino-(C₁-C₈)-alkyl, (C₂-C₈)-alkenyloximino-(C₁-C₈)-alkyl, (C₂-C₈)-alkynyloximino-(C₁-C₈)-alkyl, S(═O)ₙA¹, C(═O)A², C(═S)A², NR⁵R⁶, NR⁵—(C═O)—R⁶;
R¹, R² independently of one another are hydrogen, (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₄-C₁₀)-alka dienyl, (C₄-C₁₀)-haloalkadienyl, (C₁-C₈)-alkoxy, (C₁-C₈)-haloalkoxy, (C₂-C₈)-alkenyloxy, (C₂-C₈)-haloalkenyloxy, (C₂-C₈)-alkynyloxy, (C₂-C₈)-haloalkynyloxy, (C₃-C₈)-cycloalkyl, (C₃-C₈)-halocycloalkyl, (C₃-C₈)-cycloalkenyl, (C₃-C₈)-halocycloalkenyl, (C₃-C₈)-cycloalkoxy, (C₃-C₈)-halocycloalkoxy, (C₅-C₁₀)-bicycloalkyl, amino, (C₁-C₈)-alkylamino, di-(C₁-C₈)-alkylamino, phenyl, naphthyl or a five- or six-membered saturated, partially unsaturated or aromatic heterocycle which contains one, two, three or four heteroatoms from the group consisting of O, N and S;
R¹ and R² together with the nitrogen atom to which they are attached may also form a five- or six-membered saturated, partially unsaturated or aromatic heterocycle which may contain one, two or three further heteroatoms from the group consisting of O, N and S as ring members, where
R¹ and/or R² or a heterocycle formed from R¹ and R² may carry one, two, three or four identical or different groups R^{a} and/or two substituents attached to adjacent ring atoms may represent (C₁-C₆)-alkylene, oxy-(C₂-C₄)-alkylene or oxy-(C₁-C₃)-alkyleneoxy; wherein R^{a} is as defined below:
R^{a} is halogen, cyano, nitro, hydroxyl, carboxyl, amino, (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₄-C₁₀)-alkadienyl, (C₁-C₈)-alkoxy, (C₁-C₈)-haloalkoxy, (C₂-C₈)-alkenyloxy, (C₂-C₈)-haloalkenyloxy, (C₂-C₈)-alkynyloxy, (C₂-C₈)-haloalkynyloxy, (C₃-C₈)-cycloalkyl, (C₃-C₈)-halocycloalkyl, (C₃-C₈)-cycloalkenyl, (C₃-C₈)-cycloalkoxy, (C₃-C₈)-halocycloalkoxy, (C₃-C₈)-cycloalkenyloxy, (C₅-C₁₀)-bicycloalkyl, (C₁-C₈)-alkylcarbonyl, (C₁-C₈)-alkoxycarbonyl, (C₁-C₈)-alkylcarbonyloxy, (C₁-C₈)-alkylamino, di-(C₁-C₈)-alkylamino, (C₁-C₈)-alkylaminocarbonyl, di-(C₁-C₈)-alkylaminocarbonyl, (C₁-C₈)-alkylaminothiocarbonyl, di-(C₁-C₈)-alkylaminothiocarbonyl, oxy-(C₁-C₆)-alkyleneoxy, (C₁-C₈)-alkylthio, (C₁-C₈)-alkylsulfinyl, (C₁-C₈)-alkylsulfonyl, (C₁-C₈)-alkylsulfoxyl, phenyl, naphthyl, a five- to ten-membered saturated, partially unsaturated or aromatic heterocycle which contains one, two, three or four heteroatoms from the group consisting of O, N and S;
wherein the aliphatic, alicyclic or aromatic groups in R may carry one, two or three identical or different groups R^{b}:
R^{b} is halogen, cyano, nitro, hydroxyl, carboxyl, mercapto, amino, formyl, aminocarbonyl, aminothiocarbonyl, (C₁-C₈)-alkyl, (C₁-C₈)-haloalkyl, (C₂-C₈)-alkenyl, (C₂-C₈)-haloalkenyl, (C₄-C₁₀)-alkadienyl, (C₂-C₈)-alkynyl, (C₂-C₈)-haloalkynyl, (C₁-C₈)-alkoxy, (C₁-C₈)-haloalkoxy, (C₂-C₈)-alkenyloxy, (C₂-C₈)-alkynyloxy, (C₃-C₈)-cycloalkyl, (C₃-C₈)-cycloalkenyl, (C₃-C₈)-cycloalkoxy, (C₃-C₈)-cycloalkenyloxy, (C₅-C₁₀)-bicycloalkyl, amino, (C₁-C₈)-alkylamino, di-(C₁-C₈)-alkylamino, (C₁-C₈)-alkylcarbonyl, (C₁-C₈)-alkoxycarbonyl, (C₁-C₈)-alkylcarbonyloxy, (C₁-C₈)-alkylaminocarbonyl, di-(C₁-C₈)-alkylaminocarbonyl, (C₁-C₈)-alkylaminothiocarbonyl, di-(C₁-C₈)-alkylaminothiocarbonyl, (C₁-C₈)-alkylthio, (C₁-C₈)-alkylsulfinyl, (C₁-C₈)-alkylsulfonyl, (C₁-C₈)-alkylsulfoxyl, (C₆-C₁₄)-aryl, (C₆-C₁₄)-haloaryl, (C₆-C₁₄)-aryloxy, (C₆-C₁₄)-arylthio, (C₆-C₁₄)-aryl-(C₁-C₆)-alkoxy, (C₆-C₁₄)-aryl-(C₁-C₆)-alkyl, a five- to ten-membered saturated, partially unsaturated or aromatic heterocycle which contains one, two, three or four heteroatoms from the group consisting of O, N and S, heterocyclyloxy, heteroaryloxy, heteroarylthio, wherein the cyclic systems may be partially or fully halogenated and/or substituted by (C₁-C₈)-alkyl and/or (C₁-C₈)-haloalkyl groups;
X is (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl; cyano-(C₁-C₄)-alkyl, (C₁-C₄)-alkoxy-(C₁-C₄)-alkyl;
Y is hydrogen, halogen, cyano, (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₁-C₈)-alkoxy, (C₁-C₈)-haloalkoxy, (C₃-C₈)-cycloalkyl, (C₃-C₈)-halocycloalkyl, (C₁-C₈)-alkylthio, (C₁-C₈)-alkylsulfinyl or (C₁-C₈)-alkylsulfonyl,
wherein said method comprises:
(a2) reacting a compound of formula (IV) 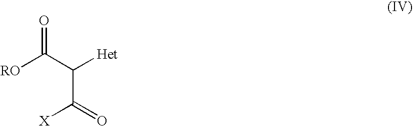
wherein Het and X are as described above, and R is (C₁-C₄)-alkyl,
with a triazole of the formula (V) 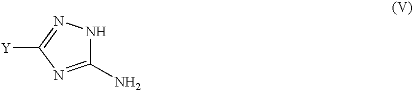
wherein a compound of formula III is prepared.

34. A process for preparing a compound of formula (I) 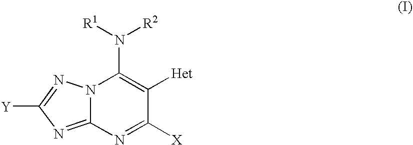
wherein,
Het is a five-membered aromatic heterocycle containing one, two, three or four heteroatoms from the group consisting of oxygen, nitrogen and sulfur, wherein Het is unsubstituted or substituted by one, two, three or four identical or different substituents L:
L is halogen, cyano, nitro, hydroxyl, cyanato (OCN), (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₄-C₁₀)-alkadienyl, (C₄-C₁₀)-haloalkadienyl, (C₁-C₈)-alkoxy, (C₁-C₈)-haloalkoxy, (C₂-C₈)-alkenyloxy, (C₂-C₈)-haloalkenyloxy, (C₂-C₈)-alkynyloxy, (C₂-C₈)-haloalkynyloxy, (C₃-C₈)-cycloalkyl, (C₃-C₈)-halocycloalkyl, (C₃-C₈)-cycloalkenyl, (C₃-C₈)-halocycloalkenyl, (C₃-C₈)-cycloalkoxy, (C₁-C₈)-alkoximino-(C₁-C₈)-alkyl, (C₂-C₈)-alkenyloximino-(C₁-C₈)-alkyl, (C₂-C₈)-alkynyloximino-(C₁-C₈)-alkyl, S(═O)ₙA¹, C(═O)A², C(═S)A², NR⁵R⁶, or NR⁵—(C═O)—R⁶; wherein
n is 0, 1 or 2;
A¹ is hydrogen, hydroxyl, (C₁-C₈)-alkyl, (C₁-C₈)-haloalkyl, amino, (C₁-C₈)-alkylamino or di-(C₁-C₈)-alkylamino, A² is one of the groups mentioned under A¹ or (C₂-C₈)-alkenyl, (C₂-C₈)-haloalkenyl, (C₂-C₈)-alkynyl, (C₂-C₈)-haloalkynyl, (C₁-C₈)-alkoxy, (C₁-C₈)-haloalkoxy, (C₂-C₈)-alkenyloxy, (C₂-C₈)-haloalkenyloxy, (C₂-C₈)-alkynyloxy, (C₂-C₈)-haloalkynyloxy, (C₃-C₈)-cycloalkoxy, or (C₃-C₈)-halocycloalkoxy;
R⁵, R⁶ independently of one another are hydrogen, (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₃-C₈)-cycloalkyl, (C₃-C₈)-halocycloalkyl, (C₃-C₈)-cycloalkenyl or (C₃-C₈)-halocycloalkenyl,
wherein the aliphatic groups of the radicals of L may carry one to four identical or different groups R^{L}:
R^{L} is halogen, cyano, (C₁-C₈)-alkoxy, (C₂-C₈)-alkenyloxy, (C₂-C₈)-alkynyloxy, (C₃-C₈)-cycloalkyl, (C₃-C₈)-cycloalkenyl, (C₃-C₈)-cycloalkyloxy, (C₃-C₈)-cycloalkenyloxy,
(C₁-C₈)-alkoximino-(C₁-C₈)-alkyl, (C₂-C₈)-alkenyloximino-(C₁-C₈)-alkyl, (C₂-C₈)-alkynyloximino-(C₁-C₈)-alkyl, S(═O)ₙA¹, C(═O)ₙA¹, C(═S)A², NR⁵R⁶, or NR⁵—(C═O)—R⁶;
R¹, R² independently of one another are hydrogen, (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₄-C₁₀)-alka dienyl, (C₄-C₁₀)-haloalkadienyl, (C₁-C₈)-alkoxy, (C₁-C₈)-haloalkoxy, (C₂-C₈)-alkenyloxy, (C₂-C₈)-haloalkenyloxy, (C₂-C₈)-alkynyloxy, (C₂-C₈)-haloalkynyloxy, (C₃-C₈)-cycloalkyl, (C₃-C₈)-halocycloalkyl, (C₃-C₈)-cycloalkenyl, (C₃-C₈)-halocycloalkenyl, (C₃-C₈)-cycloalkoxy, (C₃-C₈)-halocycloalkoxy, (C₅-C₁₀)-bicycloalkyl, amino, (C₁-C₈)-alkylamino, di-(C₁-C₈)-alkylamino, phenyl, naphthyl or a five- or six-membered saturated, partially unsaturated or aromatic heterocycle which contains one, two, three or four heteroatoms from the group consisting of O, N and S;
R¹ and R² together with the nitrogen atom to which they are attached may also form a five- or six-membered saturated, partially unsaturated or aromatic heterocycle which may contain one, two or three further heteroatoms from the group consisting of O, N and S as ring members, wherein
R¹ and/or R² or a heterocycle formed from R¹ and R² may carry one, two, three or four identical or different groups R^{a} and/or two substituents attached to adjacent ring atoms may represent (C₁-C₆)-alkylene, oxy-(C₂-C₄)-alkylene or oxy-(C₁-C₃)-alkyleneoxy; wherein R^{a} is as defined below:
R^{a} is halogen, cyano, nitro, hydroxyl, carboxyl, amino, (C₁-C₈)-alkyl, (C₁-C₉)-haloalkyl, (C₂-C₈)-alkenyl, (C₂-C₈)-haloalkenyl, (C₂-C₈)-alkynyl, (C₂-C₈)-haloalkynyl, (C₄-C₁₀)-alkadienyl, (C₁-C₈)-alkoxy, (C₁-C₈)-haloalkoxy, (C₂-C₈)-alkenyloxy, (C₂-C₈)-haloalkenyloxy, (C₂-C₈)-alkynyloxy, (C₂-C₈)-haloalkynyloxy, (C₃-C₈)-cycloalkyl, (C₃-C₈)-halocycloalkyl, (C₃-C₈)-cycloalkenyl, (C₃-C₈)-cycloalkoxy, (C₃-C₈)-halocycloalkoxy, (C₃-C₈)-cycloalkenyloxy, (C₅-C₁₀)-bicycloalkyl, (C₁-C₈)-alkylcarbonyl, (C₁-C₈)-alkoxycarbonyl, (C₁-C₈)-alkylcarbonyloxy, (C₁-C₈)-alkylamino, di-(C₁-C₈)-alkylamino, (C₁-C₈)-alkylaminocarbonyl, di-(C₁-C₈)-alkylaminocarbonyl, (C₁-C₈)-alkylaminothiocarbonyl, di-(C₁-C₈)-alkylaminothiocarbonyl, oxy-(C₁-C₆)-alkyleneoxy, (C₁-C₈)-alkylthio, (C₁-C₈)-alkylsulfinyl, (C₁-C₈)-alkylsulfonyl, (C₁-C₈)-alkylsulfoxyl, phenyl, naphthyl, a five- to ten-membered saturated, partially unsaturated or aromatic heterocycle which contains one, two, three or four heteroatoms from the group consisting of O, N and S;
wherein the aliphatic, alicyclic or aromatic groups in R^{a} may carry one, two or three identical or different groups R^{b}:
R^{b} is halogen, cyano, nitro, hydroxyl, carboxyl, mercapto, amino, formyl, aminocarbonyl, aminothiocarbonyl, (C₁-C₈)-alkyl, (C₁-C₈)-haloalkyl, (C₂-C₈)-alkenyl, (C₂-C₈)-haloalkenyl, (C₄-C₁₀)-alkadienyl, (C₂-C₈)-alkynyl, (C₂-C₈)-haloalkynyl, (C₁-C₈)-alkoxy, (C₁-C₈)-haloalkoxy, (C₂-C₈)-alkenyloxy, (C₂-C₈)-alkynyloxy, (C₃-C₈)-cycloalkyl, (C₃-C₈)-cycloalkenyl, (C₃-C₈)-cycloalkoxy, (C₃-C₈)-cycloalkenyloxy, (C₅-C₁₀)-bicycloalkyl, amino, (C₁-C₈)-alkylamino, di-(C₁-C₈)-alkylamino, (C₁-C₈)-alkylcarbonyl, (C₁-C₈)-alkoxycarbonyl, (C₁-C₈)-alkylcarbonyloxy, (C₁-C₉)-alkylaminocarbonyl, di-(C₁-C₈)-alkylaminocarbonyl, (C₁-C₈)-alkylaminothiocarbonyl, di-(C₁-C₈)-alkylaminothiocarbonyl, (C₁-C₈)-alkylthio, (C₁-C₈)-alkylsulfinyl, (C₁-C₈)-alkylsulfonyl, (C₁-C₈)-alkylsulfoxyl, (C₆-C₁₄)-aryl, (C₆-C₁₄)-haloaryl, (C₆-C₁₄)-aryloxy, (C₆-C₁₄)-arylthio, (C₆-C₁₄)-aryl-(C₁-C₆)-alkoxy, (C₆-C₁₄)-aryl-(C₁-C₆)-alkyl, a five- to ten-membered saturated, partially unsaturated or aromatic heterocycle which contains one, two, three or four heteroatoms from the group consisting of O, N and S, heterocyclyloxy, heteroaryloxy, heteroarylthio, wherein the cyclic systems may be partially or fully halogenated and/or substituted by (C₁-C₈)-alkyl and/or (C₁-C₈)-haloalkyl groups;
X is (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl; cyano-(C₁-C₄)-alkyl, or (C₁-C₄)-alkoxy-(C₁-C₄)-alkyl;
Y is hydrogen, halogen, cyano, (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₁-C₈)-alkoxy, (C₁-C₈)-haloalkoxy, (C₃-C₈)-cycloalkyl, (C₃-C₈)-halocycloalkyl, (C₁-C₈)-alkylthio, (C₁-C₈)-alkylsulfinyl or (C₁-C₈)-alkylsulfonyl;
wherein the method comprises:
(a3) reacting a compound of formula (IIa) 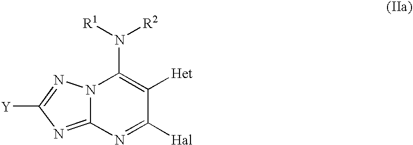
wherein R¹, R², Het, Hal and Y are as described above,
with an organometallic compound M-Z wherein M is lithium, magnesium or zinc and Z is (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, cyano-(C₁-C₄)-alkyl or (C₁-C₄)-alkoxy-(C₁-C₄)-alkyl;
wherein a compound of formula I is prepared.

35. A process for preparing a compound of formula (I) 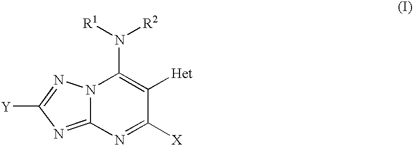
wherein,
Het is a five-membered aromatic heterocycle containing one, two, three or four heteroatoms from the group consisting of oxygen, nitrogen and sulfur, wherein Het is unsubstituted or substituted by one, two, three or four identical or different substituents L:
L is halogen, cyano, nitro, hydroxyl, cyanato (OCN), (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₄-C₁₀)-alkadienyl, (C₄-C₁₀)-haloalkadienyl, (C₁-C₈)-alkoxy, (C₁-C₈)-haloalkoxy, (C₂-C₈)-alkenyloxy, (C₂-C₈)-haloalkenyloxy, (C₂-C₈)-alkynyloxy, (C₂-C₈)-haloalkynyloxy, (C₃-C₈)-cycloalkyl, (C₃-C₈)-halocycloalkyl, (C₃-C₈)-cycloalkenyl, (C₃-C₈)-halocycloalkenyl, (C₃-C₈)-cycloalkoxy, (C₁-C₈)-alkoximino-(C₁-C₈)-alkyl, (C₂-C₈)-alkenyloximino-(C₁-C₈)-alkyl, (C₂-C₈)-alkynyloximino-(C₁-C₈)-alkyl, S(═O)ₙA¹, C(═O)A², C(═S)A², NR⁵R⁶, or NR⁵—(C═O)—R⁶; wherein
n is 0, 1 or 2;
A¹ is hydrogen, hydroxyl, (C₁-C₈)-alkyl, (C₁-C₈)-haloalkyl, amino, (C₁-C₈)-alkylamino or di-(C₁-C₈)-alkylamino, A2 is one of the groups mentioned under A¹ or (C₂-C₈)-alkenyl, (C₂-C₈)-haloalkenyl, (C₂-C₈)-alkynyl, (C₂-C₈)-haloalkynyl, (C₁-C₈)-alkoxy, (C₁-C₈)-haloalkoxy, (C₂-C₈)-alkenyloxy, (C₂-C₈)-haloalkenyloxy, (C₂-C₈)-alkynyloxy, (C₂-C₈)-haloalkynyloxy, (C₃-C₈)-cycloalkoxy, or (C₃-C₈)-halocycloalkoxy;
R⁵, R⁶ independently of one another are hydrogen, (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₃-C₈)-cycloalkyl, (C₃-C₈)-halocycloalkyl, (C₃-C₈)-cycloalkenyl or (C₃-C₈)-halocycloalkenyl,
wherein the aliphatic groups of the radicals of L may carry one to four identical or different groups R^{L}:
R^{L} is halogen, cyano, (C₁-C₈)-alkoxy, (C₂-C₈)-alkenyloxy, (C₂-C₈)-alkynyloxy, (C₃-C₈)-cycloalkyl, (C₃-C₈)-cycloalkenyl, (C₃-C₈)-cycloalkyloxy, (C₃-C₈)-cycloalkenyloxy, (C₁-C₉)-alkoximino-(C₁-C₈)-alkyl, (C₂-C₈)-alkenyloximino-(C₁-C₈)-alkyl, (C₂-C₈)-alkynyloximino-(C₁-C₈)-alkyl, S(═O)ₙA¹, C(═O)ₙA¹, C(═S)A², NR⁵R⁵, or NR⁵—(C═O)—R⁶;
R¹, R² independently of one another are hydrogen, (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₄-C₁₀)-alka dienyl, (C₄-C₁₀)-haloalkadienyl, (C₁-C₈)-alkoxy, (C₁-C₈)-haloalkoxy, (C₂-C₈)-alkenyloxy, (C₂-C₈)-haloalkenyloxy, (C₂-C₈)-alkynyloxy, (C₂-C₈)-haloalkynyloxy, (C₃-C₈)-cycloalkyl, (C₃-C₈)-halocycloalkyl, (C₃-C₈)-cycloalkenyl, (C₃-C₈)-halocycloalkenyl, (C₃-C₈)-cycloalkoxy, (C₃-C₈)-halocycloalkoxy, (C₅-C₁₀)-bicycloalkyl, amino, (C₁-C₈)-alkylamino, di-(C₁-C₈)-alkylamino, phenyl, naphthyl or a five- or six-membered saturated, partially unsaturated or aromatic heterocycle which contains one, two, three or four heteroatoms from the group consisting of O, N and S;
R¹ and R² together with the nitrogen atom to which they are attached may also form a five- or six-membered saturated, partially unsaturated or aromatic heterocycle which may contain one, two or three further heteroatoms from the group consisting of O, N and S as ring members, wherein R¹ and/or R² or a heterocycle formed from R¹ and R² may carry one, two, three or four identical or different groups R^{a} and/or two substituents attached to adjacent ring atoms may represent (C₁-C₆)-alkylene, oxy-(C₂-C₄)-alkylene or oxy-(C₁-C₃)-alkyleneoxy;
wherein R^{a} is as defined below:
R^{a} is halogen, cyano, nitro, hydroxyl, carboxyl, amino, (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₄-C₁₀)-alkadienyl, (C₁-C₈)-alkoxy, (C₁-C₈)-haloalkoxy, (C₂-C₈)-alkenyloxy, (C₂-C₈)-haloalkenyloxy, (C₂-C₈)-alkynyloxy, (C₂-C₈)-haloalkynyloxy, (C₃-C₈)-cycloalkyl, (C₃-C₈)-halocycloalkyl, (C₃-C₈)-cycloalkenyl, (C₃-C₈)-cycloalkoxy, (C₃-C₈)-halocycloalkoxy, (C₃-C₈)-cycloalkenyloxy, (C₅-C₁₀)-bicycloalkyl, (C₁-C₈)-alkylcarbonyl, (C₁-C₈)-alkoxycarbonyl, (C₁-C₈)-alkylcarbonyloxy, (C₁-C₈)-alkylamino, di-(C₁-C₈)-alkylamino, (C₁-C₈)-alkylaminocarbonyl, di-(C₁-C₈)-alkylaminocarbonyl, (C₁-C₈)-alkylaminothiocarbonyl, di-(C₁-C₈)-alkylaminothiocarbonyl, oxy-(C₁-C₆)-alkyleneoxy, (C₁-C₈)-alkylthio, (C₁-C₈)-alkylsulfinyl, (C₁-C₈)-alkylsulfonyl, (C₁-C₈)-alkylsulfoxyl, phenyl, naphthyl, a five- to ten-membered saturated, partially unsaturated or aromatic heterocycle which contains one, two, three or four heteroatoms from the group consisting of O, N and S;
wherein the aliphatic, alicyclic or aromatic groups in R^{a} may carry one, two or three identical or different groups R^{b}:
R^{b} is halogen, cyano, nitro, hydroxyl, carboxyl, mercapto, amino, formyl, aminocarbonyl, aminothiocarbonyl, (C₁-C₈)-alkyl, (C₁-C₈)-haloalkyl, (C₂-C₈)-alkenyl, (C₂-C₈)-haloalkenyl, (C₄-C₁₀)-alkadienyl, (C₂-C₈)-alkynyl, (C₂-C₈)-haloalkynyl, (C₁-C₈)-alkoxy, (C₁-C₈)-haloalkoxy, (C₂-C₈)-alkenyloxy, (C₂-C₈)-alkynyloxy, (C₃-C₈)-cycloalkyl, (C₃-C₈)-cycloalkenyl, (C₃-C₈)-cycloalkoxy, (C₃-C₈)-cycloalkenyloxy, (C₅-C₁₀)-bicycloalkyl, amino, (C₁-C₈)-alkylamino, di-(C₁-C₈)-alkylamino, (C₁-C₈)-alkylcarbonyl, (C₁-C₈)-alkoxycarbonyl, (C₁-C₈)-alkylcarbonyloxy, (C₁-C₈)-alkylaminocarbonyl, di-(C₁-C₈)-alkylaminocarbonyl, (C₁-C₈)-alkylaminothiocarbonyl, di-(C₁-C₈)-alkylaminothiocarbonyl, (C₁-C₈)-alkylthio, (C₁-C₈)-alkylsulfinyl, (C₁-C₈)-alkylsulfonyl, (C₁-C₈)-alkylsulfoxyl, (C₆-C₁₄)-aryl, (C₆-C₁₄)-haloaryl, (C₆-C₁₄)-aryloxy, (C₆-C₁₄)-arylthio, (C₆-C₁₄)-aryl-(C₁-C₆)-alkoxy, (C₆-C₁₄)-aryl-(C₁-C₆)-alkyl, a five- to ten-membered saturated, partially unsaturated or aromatic heterocycle which contains one, two, three or four heteroatoms from the group consisting of O, N and S, heterocyclyloxy, heteroaryloxy, heteroarylthio, wherein the cyclic systems may be partially or fully halogenated and/or substituted by (C₁-C₈)-alkyl and/or (C₁-C₈)-haloalkyl groups;
X is (C₁-C₈)-alkyl;
Y is hydrogen, halogen, cyano, (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₁-C₈)-alkoxy, (C₁-C₈)-haloalkoxy, (C₃-C₈)-cycloalkyl, (C₃-C₈)-halocycloalkyl, (C₁-C₈)-alkylthio, (C₁-C₈)-alkylsulfinyl or (C₁-C₈)-alkylsulfonyl;
wherein the method comprises:
(a) reacting a compound of formula (IIa) 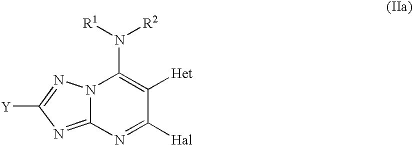
wherein Hal, Het, R¹, R², and Y are as described above;
with a malonate compound of formula (IVa) 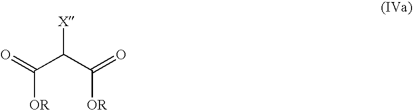
to produce a compound of formula (VI) 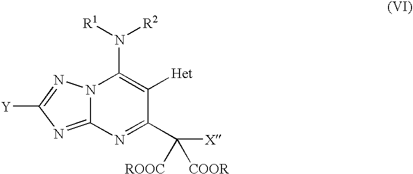
wherein X″ is hydrogen or (C₁-C₇)-alkyl and R is (C₁-C₄)-alkyl and Het, R¹, R² and Y are as defined above; and
(b) hydrolyzing compound (VI); and
(c) decarboxylation of the hydrolysis product obtained in step (b);
wherein a compound of formula I is prepared.

36. A compound of the formula (II) 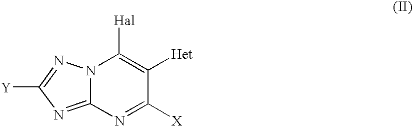
Hal is halogen and
Het is a five-membered aromatic heterocycle containing one, two, three or four heteroatoms from the group consisting of oxygen, nitrogen and sulfur, wherein Het is unsubstituted or substituted by one, two, three or four identical or different substituents L:
L is halogen, cyano, nitro, hydroxyl, cyanato (OCN), (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₄-C₁₀)-alkadienyl, (C₄-C₁₀)-haloalkadienyl, (C₁-C₈)-alkoxy, (C₁-C₈)-haloalkoxy, (C₂-C₈)-alkenyloxy, (C₂-C₈)-haloalkenyloxy, (C₂-C₈)-alkynyloxy, (C₂-C₈)-haloalkynyloxy, (C₃-C₈)-cycloalkyl, (C₃-C₈)-halocycloalkyl, (C₃-C₈)-cycloalkenyl, (C₃-C₈)-halocycloalkenyl, (C₃-C₈)-cycloalkoxy, (C₁-C₈)-alkoximino-(C₁-C₈)-alkyl, (C₂-C₈)-alkenyloximino-(C₁-C₈)-alkyl, (C₂-C₈)-alkynyloximino-(C₁-C₈)-alkyl, S(═O)ₙA¹, C(═O)A², C(═S)A², NR⁵R⁶, NR⁵—(C═O)—R⁶; where
n is 0, 1 or 2;
A¹ is hydrogen, hydroxyl, (C₁-C₈)-alkyl, (C₁-C₈)-haloalkyl, amino, (C₁-C₈)-alkylamino or di-(C₁-C₈)-alkylamino, A2 is one of the groups mentioned under A¹ or (C₂-C₈)-alkenyl, (C₂-C₈)-haloalkenyl, (C₂-C₈)-alkynyl, (C₂-C₈)-haloalkynyl, (C₁-C₈)-alkoxy, (C₁-C₈)-haloalkoxy, (C₂-C₈)-alkenyloxy, (C₂-C₈)-haloalkenyloxy, (C₂-C₈)-alkynyloxy, (C₂-C₈)-haloalkynyloxy, (C₃-C₈)-cycloalkoxy, (C₃-C₈)-halocycloalkoxy;
R⁵, R⁶ independently of one another are hydrogen, (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₃-C₈)-cycloalkyl, (C₃-C₈)-halocycloalkyl, (C₃-C₈)-cycloalkenyl or (C₃-C₈)-halocycloalkenyl,
wherein the aliphatic groups of the radicals of L may carry one to four identical or different groups R^{L}:
R^{L} is halogen, cyano, (C₁-C₈)-alkoxy, (C₂-C₈)-alkenyloxy, (C₂-C₈)-alkynyloxy, (C₃-C₈)-cycloalkyl, (C₃-C₈)-cycloalkenyl, (C₃-C₈)-cycloalkyloxy, (C₃-C₈)-cycloalkenyloxy, (C₁-C₈)-alkoximino-(C₁-C₈)-alkyl, (C₂-C₈)-alkenyloximino-(C₁-C₈)-alkyl, (C₂-C₈)-alkynyloximino-(C₁-C₈)-alkyl, S(═O)ₙA¹, C(═O)A², C(═S)ₙA¹, NR⁵R⁶, NR⁵—(C═O)—R;
X is (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl; cyano-(C₁-C₄)-alkyl, (C₁-C₄)-alkoxy-(C₁-C₄)-alkyl; and
Y is hydrogen, halogen, cyano, (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₁-C₈)-alkoxy, (C₁-C₈)-haloalkoxy, (C₃-C₈)-cycloalkyl, (C₃-C₈)-halocycloalkyl, (C₁-C₈)-alkylthio, (C₁-C₈)-alkylsulfinyl or (C₁-C₈)-alkylsulfonyl.

37. A compound of formula (III) 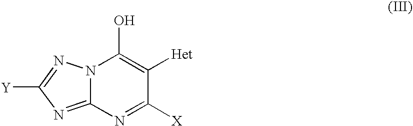
wherein
Het is a five-membered aromatic heterocycle containing one, two, three or four heteroatoms from the group consisting of oxygen, nitrogen and sulfur, wherein Het is unsubstituted or substituted by one, two, three or four identical or different substituents L:
L is halogen, cyano, nitro, hydroxyl, cyanato (OCN), (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₄-C₁₀)-alkadienyl, (C₄-C₁₀)-haloalkadienyl, (C₁-C₈)-alkoxy, (C₁-C₈)-haloalkoxy, (C₂-C₈)-alkenyloxy, (C₂-C₈)-haloalkenyloxy, (C₂-C₈)-alkynyloxy, (C₂-C₈)-haloalkynyloxy, (C₃-C₈)-cycloalkyl, (C₃-C₈)-halocycloalkyl, (C₃-C₈)-cycloalkenyl, (C₃-C₈)-halocycloalkenyl, (C₃-C₈)-cycloalkoxy, (C₁-C₈)-alkoximino-(C₁-C₈)-alkyl, (C₂-C₈)-alkenyloximino-(C₁-C₈)-alkyl, (C₂-C₈)-alkynyloximino-(C₁-C₈)-alkyl, S(═O)ₙA¹, C(═O)A², C(═S)A², NR⁵R⁶, NR⁵—(C═O)—R⁶; where
n is 0, 1 or 2;
A¹ is hydrogen, hydroxyl, (C₁-C₈)-alkyl, (C₁-C₈)-haloalkyl, amino, (C₁-C₈)-alkylamino or di-(C₁-C₈)-alkylamino, A² is one of the groups mentioned under A¹ or (C₂-C₈)-alkenyl, (C₂-C₈)-haloalkenyl, (C₂-C₈)-alkynyl, (C₂-C₈)-haloalkynyl, (C₁-C₈)-alkoxy, (C₁-C₈)-haloalkoxy, (C₂-C₈)-alkenyloxy, (C₂-C₈)-haloalkenyloxy, (C₂-C₈)-alkynyloxy, (C₂-C₈)-haloalkynyloxy, (C₃-C₈)-cycloalkoxy, (C₃-C₈)-halocycloalkoxy;
R⁵, R⁶ independently of one another are hydrogen, (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₃-C₈)-cycloalkyl, (C₃-C₈)-halocycloalkyl, (C₃-C₈)-cycloalkenyl or (C₃-C₈)-halocycloalkenyl,
wherein the aliphatic groups of the radicals of L may carry one to four identical or different groups R^{L}:
R^{L} is halogen, cyano, (C₁-C₈)-alkoxy, (C₂-C₈)-alkenyloxy, (C₂-C₈)-alkynyloxy, (C₃-C₈)-cycloalkyl, (C₃-C₈)-cycloalkenyl, (C₃-C₈)-cycloalkyloxy, (C₃-C₈)-cycloalkenyloxy, (C₁-C₈)-alkoximino-(C₁-C₈)-alkyl, (C₂-C₈)-alkenyloximino-(C₁-C₈)-alkyl, (C₂-C₈)-alkynyloximino-(C₁-C₈)-alkyl, S(═O)ₙA¹, C(═O)A², C(═S)A², NR⁵R⁶, NR⁵—(C═O)—R;
X is (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl; cyano-(C₁-C₄)-alkyl, (C₁-C₄)-alkoxy-(C₁-C₄)-alkyl;
Y is hydrogen, halogen, cyano, (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₁-C₈)-alkoxy, (C₁-C₈)-haloalkoxy, (C₃-C₈)-cycloalkyl, (C₃-C₈)-halocycloalkyl, (C₁-C₈)-alkylthio, (C₁-C₈)-alkylsulfinyl or (C₁-C₈)-alkylsulfonyl.

38. A compound of formula (IV) 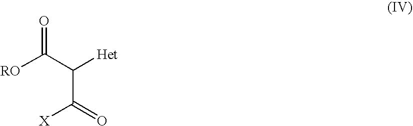
wherein
Het is a five-membered aromatic heterocycle containing one, two, three or four heteroatoms from the group consisting of oxygen, nitrogen and sulfur, wherein Het is unsubstituted or substituted by one, two, three or four identical or different substituents L:
L is halogen, cyano, nitro, hydroxyl, cyanato (OCN), (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₄-C₁₀)-alkadienyl, (C₄-C₁₀)-haloalkadienyl, (C₁-C₈)-alkoxy, (C₁-C₈)-haloalkoxy, (C₂-C₈)-alkenyloxy, (C₂-C₈)-haloalkenyloxy, (C₂-C₈)-alkynyloxy, (C₂-C₈)-haloalkynyloxy, (C₃-C₈)-cycloalkyl, (C₃-C₈)-halocycloalkyl, (C₃-C₈)-cycloalkenyl, (C₃-C₈)-halocycloalkenyl, (C₃-C₈)-cycloalkoxy, (C₁-C₈)-alkoximino-(C₁-C₈)-alkyl, (C₂-C₈)-alkenyloximino-(C₁-C₈)-alkyl, (C₂-C₈)-alkynyloximino-(C₁-C₈)-alkyl, S(═O)ₙA¹, C(═O)A², C(═S)A², NR⁵R⁶, NR⁵—(C═O)—R⁶; where
n is 0, 1 or 2;
A¹ is hydrogen, hydroxyl, (C₁-C₈)-alkyl, (C₁-C₈)-haloalkyl, amino, (C₁-C₈)-alkylamino or di-(C₁-C₈)-alkylamino, A2 is one of the groups mentioned under A¹ or (C₂-C₈)-alkenyl, (C₂-C₈)-haloalkenyl, (C₂-C₈)-alkynyl, (C₂-C₈)-haloalkynyl, (C₁-C₈)-alkoxy, (C₁-C₈)-haloalkoxy, (C₂-C₈)-alkenyloxy, (C₂-C₈)-haloalkenyloxy, (C₂-C₈)-alkynyloxy, (C₂-C₈)-haloalkynyloxy, (C₃-C₈)-cycloalkoxy, (C₃-C₈)-halocycloalkoxy;
R⁵, R⁶ independently of one another are hydrogen, (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₃-C₈)-cycloalkyl, (C₃-C₈)-halocycloalkyl, (C₃-C₈)-cycloalkenyl or (C₃-C₈)-halocycloalkenyl,
wherein the aliphatic groups of the radicals of L may carry one to four identical or different groups R^{L}:
R^{L} is halogen, cyano, (C₁-C₈)-alkoxy, (C₂-C₈)-alkenyloxy, (C₂-C₈)-alkynyloxy, (C₃-C₈)-cycloalkyl, (C₃-C₈)-cycloalkenyl, (C₃-C₈)-cycloalkyloxy, (C₃-C₈)-cycloalkenyloxy, (C₁-C₈)-alkoximino-(C₁-C₈)-alkyl, (C₂-C₈)-alkenyloximino-(C₁-C₈)-alkyl, (C₂-C₈)-alkynyloximino-(C₁-C₈)-alkyl, S(═O)ₙA¹, C(═O)A², C(═S)A², NR⁵R⁶, NR⁵—(C═O)—R⁶;
X is (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl; cyano-(C₁-C₄)-alkyl, (C₁-C₄)-alkoxy-(C₁-C₄)-alkyl;
and R is (C₁-C₄)-alkyl.

39. A compound of formula (VI) 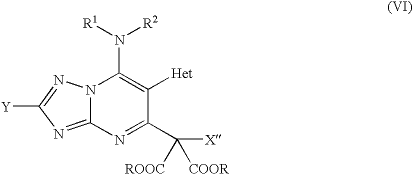
wherein X″ is hydrogen or (C₁-C₇)-alkyl and R is (C₁-C₄)-alkyl;
Het is a five-membered aromatic heterocycle containing one, two, three or four heteroatoms from the group consisting of oxygen, nitrogen and sulfur, wherein Het is unsubstituted or substituted by one, two, three or four identical or different substituents L:
L is halogen, cyano, nitro, hydroxyl, cyanato (OCN), (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₄-C₁₀)-alkadienyl, (C₄-C₁₀)-haloalkadienyl, (C₁-C₈)-alkoxy, (C₁-C₈)-haloalkoxy, (C₂-C₈)-alkenyloxy, (C₂-C₈)-haloalkenyloxy, (C₂-C₈)-alkynyloxy, (C₂-C₈)-haloalkynyloxy, (C₃-C₈)-cycloalkyl, (C₃-C₈)-halocycloalkyl, (C₃-C₈)-cycloalkenyl, (C₃-C₈)-halocycloalkenyl, (C₃-C₈)-cycloalkoxy, (C₁-C₈)-alkoximino-(C₁-C₈)-alkyl, (C₂-C₈)-alkenyloximino-(C₁-C₈)-alkyl, (C₂-C₈)-alkynyloximino-(C₁-C₈)-alkyl, S(═O)ₙA¹, C(═O)A², C(═S)A², NR⁵R⁶, NR⁵—(C═O)—R⁶; where
n is 0, 1 or 2;
A is hydrogen, hydroxyl, (C₁-C₈)-alkyl, (C₁-C₈)-haloalkyl, amino, (C₁-C₈)-alkylamino or di-(C₁-C₈)-alkylamino, A2 is one of the groups mentioned under A¹ or (C₂-C₈)-alkenyl, (C₂-C₈)-haloalkenyl, (C₂-C₈)-alkynyl, (C₂-C₈)-haloalkynyl, (C₁-C₈)-alkoxy, (C₁-C₈)-haloalkoxy, (C₂-C₈)—alkenyloxy, (C₂-C₈)-haloalkenyloxy, (C₂-C₈)-alkynyloxy, (C₂-C₈)-haloalkynyloxy, (C₃-C₈)-cycloalkoxy, (C₃-C₈)-halocycloalkoxy;
R⁵, R⁶ independently of one another are hydrogen, (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₃-C₈)-cycloalkyl, (C₃-C₈)-halocycloalkyl, (C₃-C₈)-cycloalkenyl or (C₃-C₈)-halocycloalkenyl,
wherein the aliphatic groups of the radicals of L may carry one to four identical or different groups R^{L}:
R^{L} is halogen, cyano, (C₁-C₈)-alkoxy, (C₂-C₈)-alkenyloxy, (C₂-C₈)-alkynyloxy, (C₃-C₈)-cycloalkyl, (C₃-C₈)-cycloalkenyl, (C₃-C₈)-cycloalkyloxy, (C₃-C₈)-cycloalkenyloxy, (C₁-C₈)-alkoximino-(C₁-C₈)-alkyl, (C₂-C₈)-alkenyloximino-(C₁-C₈)-alkyl, (C₂-C₈)-alkynyloximino-(C₁-C₈)-alkyl, S(═O)ₙA¹, C(═O)A², C(═S)A², NR⁵R⁶, NR⁵—(C═O)—R⁶;
R¹, R² independently of one another are hydrogen, (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₄-C₁₀)-alka dienyl, (C₄-C₁₀)-haloalkadienyl, (C₁-C₈)-alkoxy, (C₁-C₈)-haloalkoxy, (C₂-C₈)-alkenyloxy, (C₂-C₈)-haloalkenyloxy, (C₂-C₈)-alkynyloxy, (C₂-C₈)-haloalkynyloxy, (C₃-C₈)-cycloalkyl, (C₃-C₈)-halocycloalkyl, (C₃-C₈)-cycloalkenyl, (C₃-C₈)-halocycloalkenyl, (C₃-C₈)-cycloalkoxy, (C₃-C₈)-halocycloalkoxy, (C₅-C₁₀)-bicycloalkyl, amino, (C₁-C₈)-alkylamino, di-(C₁-C₈)-alkylamino, phenyl, naphthyl or a five- or six-membered saturated, partially unsaturated or aromatic heterocycle which contains one, two, three or four heteroatoms from the group consisting of O, N and S;
R¹ and R² together with the nitrogen atom to which they are attached may also form a five- or six-membered saturated, partially unsaturated or aromatic heterocycle which may contain one, two or three further heteroatoms from the group consisting of O, N and S as ring members, where
R¹ and/or R² or a heterocycle formed from R¹ and R² may carry one, two, three or four identical or different groups R^{a} and/or two substituents attached to adjacent ring atoms may represent (C₁-C₆)-alkylene, oxy-(C₂-C₄)-alkylene or oxy-(C₁-C₃)-alkyleneoxy; wherein R^{a} is as defined below:
R^{a} is halogen, cyano, nitro, hydroxyl, carboxyl, amino, (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₄-C₁₀)-alkadienyl, (C₁-C₈)-alkoxy, (C₁-C₈)-haloalkoxy, (C₂-C₈)-alkenyloxy, (C₂-C₈)-haloalkenyloxy, (C₂-C₈)-alkynyloxy, (C₂-C₈)-haloalkynyloxy, (C₃-C₈)-cycloalkyl, (C₃-C₈)-halocycloalkyl, (C₃-C₈)-cycloalkenyl, (C₃-C₈)-cycloalkoxy, (C₃-C₈)-halocycloalkoxy, (C₃-C₈)-cycloalkenyloxy, (C₅-C₁₀)-bicycloalkyl, (C₁-C₈)-alkylcarbonyl, (C₁-C₈)-alkoxycarbonyl, (C₁-C₈)-alkylcarbonyloxy, (C₁-C₈)-alkylamino, di-(C₁-C₈)-alkylamino, (C₁-C₈)-alkylaminocarbonyl, di-(C₁-C₈)-alkylaminocarbonyl, (C₁-C₈)-alkylaminothiocarbonyl, di-(C₁-C₈)-alkylaminothiocarbonyl, oxy-(C₁-C₆)-alkyleneoxy, (C₁-C₈)-alkylthio, (C₁-C₈)-alkylsulfinyl, (C₁-C₈)-alkylsulfonyl, (C₁-C₈)-alkylsulfoxyl, phenyl, naphthyl, a five- to ten-membered saturated, partially unsaturated or aromatic heterocycle which contains one, two, three or four heteroatoms from the group consisting of O, N and S;
wherein the aliphatic, alicyclic or aromatic groups in R^{a} may carry one, two or three identical or different groups R^{b}:
R^{b} is halogen, cyano, nitro, hydroxyl, carboxyl, mercapto, amino, formyl, aminocarbonyl, aminothiocarbonyl, (C₁-C₈)-alkyl, (C₁-C₈)-haloalkyl, (C₂-C₈)-alkenyl, (C₂-C₈)-haloalkenyl, (C₄-C₁₀)-alkadienyl, (C₂-C₈)-alkynyl, (C₂-C₈)-haloalkynyl, (C₁-C₈)-alkoxy, (C₁-C₈)-haloalkoxy, (C₂-C₈)-alkenyloxy, (C₂-C₈)-alkynyloxy, (C₃-C₈)-cycloalkyl, (C₃-C₈)-cycloalkenyl, (C₃-C₈)-cycloalkoxy, (C₃-C₈)-cycloalkenyloxy, (C₅-C₁₀)-bicycloalkyl, amino, (C₁-C₈)-alkylamino, di-(C₁-C₈)-alkylamino, (C₁-C₈)-alkylcarbonyl, (C₁-C₈)-alkoxycarbonyl, (C₁-C₈)-alkylcarbonyloxy, (C₁-C₈)-alkylaminocarbonyl, di-(C₁-C₈)-alkylaminocarbonyl, (C₁-C₈)-alkylaminothiocarbonyl, di-(C₁-C₈)-alkylaminothiocarbonyl, (C₁-C₈)-alkylthio, (C₁-C₈)-alkylsulfinyl, (C₁-C₈)-alkylsulfonyl, (C₁-C₈)-alkylsulfoxyl, (C₆-C₁₄)-aryl, (C₆-C₁₄)-haloaryl, (C₆-C₁₄)-aryloxy, (C₆-C₁₄)-arylthio, (C₆-C₁₄)-aryl-(C₁-C₆)-alkoxy, (C₆-C₄)-aryl-(C₁-C₆)-alkyl, a five- to ten-membered saturated, partially unsaturated or aromatic heterocycle which contains one, two, three or four heteroatoms from the group consisting of O, N and S, heterocyclyloxy, heteroaryloxy, heteroarylthio, wherein the cyclic systems may be partially or fully halogenated and/or substituted by (C₁-C₈)-alkyl and/or (C₁-C₈)-haloalkyl groups;
Y is hydrogen, halogen, cyano, (C₁-C₈)-alkyl, (C₁-C₈)-haloalkyl, (C₂-C₈)-alkenyl, (C₂-C₈)-haloalkenyl, (C₂-C₈)-alkynyl, (C₂-C₈)-haloalkynyl, (C₁-C₈)-alkoxy, (C₁-C₈)-haloalkoxy, (C₃-C₈)-cycloalkyl, (C₃-C₈)-halocycloalkyl, (C₁-C₈)-alkylthio, (C₁-C₈)-alkylsulfinyl or (C₁-C₈)-alkylsulfonyl.
